(19) United States
(12) Patent Application Publication
(10) Pub. No.: US 2024/0263147 A1
(43) Pub. Date: Aug. 8, 2024

(54) CARDIAC ORGANOID AND ANTI-ELECTRO-MITOCHONDRIAL DESYNCHRONIZATION THERAPY

(71) Applicant: YISSUM RESEARCH DEVELOPMENT COMPANY OF THE HEBREW UNIVERSITY OF JERUSALEM LTD., Jerusalem (IL)

(72) Inventors: Yaakov NAHMIAS, Mevasert Zion (IL); Mohammad GHOSHEH, Jerusalem (IL); Avner EHRLICH, Jerusalem (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/598,152

(22) Filed: Mar. 7, 2024

Related U.S. Application Data
(63) Continuation of application No. PCT/IL2022/050982, filed on Sep. 8, 2022.
(60) Provisional application No. 63/242,091, filed on Sep. 9, 2021.
(60) Provisional application No. 63/329,448, filed on Apr. 10, 2022.

(51) Int. Cl. C12N 5/071; C12N 5/00; C12N 5/077; G01N 33/50

(52) U.S. Cl. CPC C12N 5/0697; C12N 5/0062; C12N 5/0657; C12N 5/069; G01N 33/5088; C12N 2513/00

(57) ABSTRACT

The present invention provides a method of treating a disease or disorder characterized by electro-mitochondrial desynchronization in a subject in need thereof, comprising confirming the disease or disorder is characterized by electro-mitochondrial desynchronization in the subject and administering an agent that modulates mitochondrial calcium concentration and/or increases mitochondrial calcium channel activity in a tissue of the disease or disorder in the subject. Multichambered cardiac organoids comprising cardiomyocytes and endothelial cells and at least two chambers beating in synchrony are provided. Further provided are methods of using the multichambered cardiac organoid, methods of producing a cardiac organoid. Systems for making measurements within cellular aggregates or tissues and the use of same for testing therapeutic agents is also provided.

### CROSS-REFERENCE TO RELATED APPLICATIONS

This application is a Bypass Continuation of PCT Patent Application No. PCT/IL2022/050982 having International Filing date of Sep. 8, 2022, which claims the benefit of priority of U.S. Provisional Patent Application Nos. 63/242,091 filed Sep. 9, 2021, and 63/329,448 filed Apr. 10, 2022, the contents of which are all incorporated herein by reference in their entirety.

### FIELD OF INVENTION

The present invention relates to cardiac organoids, including methods of using same.

### REFERENCE TO AN ELECTRONIC SEQUENCE LISTING

The contents of the electronic sequence listing (HUJI-P-078-PCT SQL; size: 4,445 bytes; and date of creation: Sep. 8, 2022) is herein incorporated by reference in its entirety.

### BACKGROUND

Cardiovascular diseases are the leading cause of death worldwide affecting nearly half of the adult population of the United States. Recent studies led to a growing appreciation for the contribution of cardiomyocyte metabolism to disease progression showing metabolic changes occurring during heart failure and arrhythmogenesis. These insights elucidated the mechanism of action of established cardiac treatments such as β-blockers and led to the development of several therapeutics, including elamipretide, that target metabolic pathways despite an incomplete understanding of the dynamics of such interventions.

The electromechanical rhythms of the cardiac muscle have long been hypothesized to drive cyclic changes in cardiomyocyte metabolism. Alterations in cellular metabolism due to dyslipidemia or insulin resistance are thought to contribute to abnormal ionic homeostasis that increases susceptibility to arrhythmogenic events. Regretfully, differences in ion channel dynamics, contraction rate, and metabolism often frustrate our ability to translate findings from small animal models to patients. These differences between animal and human models result in a distinctive response to pathological events on the molecular and metabolic levels.

Human induced pluripotent stem cells (hiPSCs) derived cardiomyocytes offer a more relevant model of human cardiac metabolism and physiology. Recent work demonstrated the utility of transitioning to three dimensional (3D) cardiac tissue, resulting in a more mature tissue function, and a higher structural complexity that captures critical aspects of cardiac metabolism. Other works increased tissue complexity by adding endothelial vascularization, an epicardial cell layer, cardiac fibroblasts, or an internal cavity. In parallel, several groups developed constructs that allow real-time sensing of contraction dynamics using heteropolar wires, 3D-printed strain sensors, and microelectrode arrays that offer new opportunities to study aspects of cardiac physiology in a human-relevant system.

There is still a great need for microphysiological metamaterials, combining cells and sensors, such as for advancing the understanding of human physiology, inclusive of the behavior of cardiomyocytes.

### SUMMARY

The project leading to this application has received funding from the European Research Council (ERC) under the European Union's Horizon 2020 research and innovation program (grant agreement No [681870]).

The present invention, in some embodiments, is based, in part, on the finding that vascularization of cardiac organoids produced anisotropic stress resulting in complex multi-chambered structures. These cardiac organoids include pacemaker-like cell clusters, fibroblasts, epicardial shell, and endocardial lining while exhibiting in vivo-like gene expression and function. Integration of the herein disclosed cardiac organoids in a dual-photomultiplier tubes (PMT) sensor platform permitted simultaneous real-time measurements of oxygen, field potential, and contraction with >10 Hz resolution.

Metabolic imbalances are important drivers and markers of cardiac disease. However, current methods to measure metabolism are slow taking minutes to hours to quantify metabolic fluxes such as glycolysis, mitochondrial respiration or fatty acid oxidation. Therefore, drug development is limited to understanding and treating systemic metabolic disease in a context of cardiac health. As nonlimiting examples, high glucose or dyslipidemia are chronic conditions that eventually affect cardiac function, and thus their treatment with drugs like empagliflozin or statins is indicated. The electro-metabolic-mechanical sensing method developed in this application allows for rapid measurement of metabolic fluxes that control cardiac rhythms. Indeed, arrhythmia occurs in 25% of the adults over the age of 40 and its exact causes are unclear. Thus, method described in this patent can be used to develop new drug and therapeutics for the treatment of multiple types of cardiac arrhythmia as well as ischemic injury.

Simultaneous electro-metabolic-mechanical sensing allowed the inventors to demonstrate that mitochondrial function in human cardiac organoids is synchronized to their electrical activity, rather than their mechanical action, as previously theorized. The inventors demonstrated that any type of inhibition of the mitochondrial calcium uniporter (MCU) would cause arrhythmia and that this arrhythmia can be reversed by either (1) blocking the interaction of the drug with the MCU protein, or by (2) increasing MCU activity either directly or indirectly.

As a nonlimiting example the inventors showed that the inhibition of mitochondrial calcium uniporter by the chemotherapeutic mitoxantrone disturbed this electro-mitochondrial coupling, thereby resulting in arrhythmia. The inventors have partly reversed this effect by co-administration of metformin, which indirectly activated the MCU thereby suggesting a combination therapy that could block chemotherapy-induced arrhythmia.

Chemotherapy-induced arrhythmia is a complication of cancer treatment that results in significantly increased morbidity and mortality. Atrial fibrillation, ventricular ectopic beats, and prolonged QTc are the most common arrhythmias suffered by cancer patients undergoing chemotherapy. The mechanism of chemotherapy-induced arrhythmia is poorly understood, until the inventors demonstrated it is caused by a disruption to mitochondrial metabolism using the Simultaneous electro-metabolic-mechanical sensing platform.

According to a first aspect, there is provided a method of treating a disease or disorder characterized by electro-mitochondrial desynchronization in a subject in need thereof, the method comprising confirming that the disease or disorder is characterized by electro-mitochondrial desynchronization in the subject and administering to the subject a therapeutically effective amount of an agent capable of
a. modulating mitochondrial calcium concentration in a tissue of the disease or disorder in the subject;
b. modulating mitochondrial calcium channel activity in the tissue; or
c. a combination there thereof;
   thereby treating a disease or disorder characterized by electro-mitochondrial desynchronization.

According to some embodiments, the desynchronization comprises decreased mitochondrial calcium concentration or mitochondrial calcium channel activity as compared to a healthy control and the modulating is increasing or wherein the desynchronization comprises increased mitochondrial calcium concentration or mitochondrial calcium channel activity as compared to a healthy control and the modulating is decreasing.

According to some embodiments, the modulating mitochondrial calcium concentration and/or mitochondrial calcium channel activity comprises modulating mitochondrial calcium uniporter (MCU) activity.

According to some embodiments, the modulating comprises administering an agent selected from metformin, kaempferol, spermine, A-769662, AICAR, IND 1316, PF 06409577, ZLN 024, Erastin, Honokiol, Ezetimibe, Disulfiram, Efsevin and spermidine.

According to some embodiments, the disease or disorder is selected from the group consisting of: arrhythmia, cardiomyopathy, seizures, epilepsy, motor neuron spasms, muscle weakness, muscular atrophy, a channelopathy, Catecholaminergic polymorphic ventricular tachycardia (CPVT), myopathy with extrapyramidal signs (MPXPS), Alzheimer's disease, Huntington's disease, Parkinson's disease, amyotrophic lateral sclerosis (AML), hereditary spastic paraplegia, ischemia-reperfusion injury, ischemic heart disease, rare mitochondrial encephalomyopathy, Sagittal Sinus Thrombosis, Intracranial Sinus Thrombosis, Stormorken Syndrome, Generalized Epilepsy With Febrile Seizures Plus, Optic Atrophy 3, Autosomal Dominant, Generalized Epilepsy With Febrile Seizures Plus, Type 6, Palmoplantar Keratoderma, Nonepidermolytic, and Eastern Equine Encephalitis; optionally wherein the arrhythmia is a cancer treatment induced arrhythmia (CTIA).

According to some embodiments, the desynchronization comprises increased mitochondrial calcium concentration or mitochondrial calcium channel activity and the disease or disorder is selected from mitochondrial encephalomyopathy, Sagittal Sinus Thrombosis, Intracranial Sinus Thrombosis, Stormorken Syndrome, Generalized Epilepsy With Febrile Seizures Plus, Optic Atrophy 3, Autosomal Dominant, Generalized Epilepsy With Febrile Seizures Plus, Type 6, Palmoplantar Keratoderma, Nonepidermolytic, Eastern Equine Encephalitis and CTIA, optionally wherein the cancer treatment is doxorubicin.

According to some embodiments, the desynchronization comprises decreased mitochondrial calcium concentration or mitochondrial calcium channel activity and the disease or disorder is selected from arrhythmia, cardiomyopathy, seizures, epilepsy, motor neuron spasms, muscle weakness, muscular atrophy, a channelopathy, CPVT, MPXPS, Alzheimer's disease, Huntington's disease, Parkinson's disease, AML, hereditary spastic paraplegia, ischemia-reperfusion injury, ischemic heart disease, rare and CTIA.

According to some embodiments, the disease is CPVT and the modulating comprises administering an agent selected from Spermine, Spermidine, Metformin, Erastin, A-769662, AICAR, IND 1316, PF 06409577 and ZLN 024.

According to some embodiments, the disease or disorder is caused by the administration of an agent that causes a cardiac side effect and wherein the agent is selected from a calcium signaling targeting agent, a calcium channel blocker, and an antineoplastic agent, optionally wherein the agent is selected from the agents provided in Table 1.

According to some embodiments, the confirming comprises at least one of:
a. determining mitochondrial calcium concentration in a sample obtained from the subject and wherein a concentration beyond a predetermined threshold indicates desynchronization, optionally wherein the predetermined threshold is calcium concentration in a healthy subject or in a subject suffering from the disease or disorder not characterized by electro-mitochondrial desynchronization;
b. observing in the patient arrhythmic or proarrhythmic symptoms that do not respond to antiarrhythmic treatment that targets electrical activity through membrane channels thus indicating desynchronization, optionally wherein the antiarrhythmic treatments are selected from: sodium channel blockers, beta blockers, potassium channel blockers, non-dihydropyridine calcium channel blockers, adenosine and digoxin;
c. an abnormal readout indicates desynchronization, optionally wherein an abnormal readout comprises at least one of late potentials, reduced R waves, and increased R/R ratios;
d. confirming exposure to an agent that is known to produce electro-mitochondrial desynchronization, optionally wherein the agent is an agent selected from those provided in Table 1 and toluene, trichloroethane, xylene, heptanes, hexane, ethyl ether trichloroethylene, trichlorotrifluoroethane, carbon monoxide, carbon disulfide, pesticides, bisphenol A (BPA), methane-derived halogenated hydrocarbons, organic nitrates, arsenic, cadmium, cobalt, organic solvents, and metals; and
e. confirming a medical history containing diagnosis of symptoms that are indicative of electro-mitochondrial desynchronization.

According to another aspect, there is provided a multichambered cardiac organoid comprising cardiomyocytes and endothelial cells and at least two chambers beating in synchrony.

According to some embodiments, all chambers beat in synchrony or wherein the organoid produces a biphasic beating.

According to some embodiments, the organoid comprises pacemaker-like cell clusters, optionally wherein the pacemaker-like cell clusters are Potassium/sodium hyperpolarization-activated cyclic nucleotide-gated channel 4 (HCN4) and Short-stature homeobox 2 (SHOX2) positive.

According to some embodiments, the organoid comprises an outer epicardium, optionally wherein the epicardium comprises cells positive for Wilms' tumor-1 (WT1) and T-box transcription factor 18 (TBX18).

According to some embodiments, the organoid comprises an inner endocardium, optionally wherein the endocardium comprises cells positive for Platelet endothelial cell adhesion molecule (PECAM-1).

According to some embodiments, the synchronized beating comprises a basal beating frequency of between 50 and 90 beats per minute (bpm).

According to some embodiments, the organoid comprises vascular structures, circumferentially aligned cardiomyocytes surrounding hollow chambers, elongated cardiomyocytes organized in a sarcomeric pattern, capillaries within a wall of the chambers and cardiac fibroblast-like cells, optionally wherein the fibroblast-like cells are Periostin (POSTN) and/or Vimentin positive.

According to some embodiments, the organoid comprises at least one parameter that is increased as compared to isolated cardiomyocytes in culture or fetal cardiac tissue in culture, wherein the parameter is selected from basal respiration, oxidative phosphorylation, mitochondrial maximal capacity and expression of at least factor selected from the group consisting of: TNNT2, TNNI3, Cx43, MYH7, AKAP6, GJA5, JPH2, SLC8A1, ATP2A2, CACNA1C, RYR2, CASQ2, PLN, CAMK2B, TRDN, CAV3, BIN1, AMP2, SCN5A, KIR2.1, ITPR3, HCN2, SCN1B, HCN1, KCNJ8, KCNH2, PRKAA1, CPT1A, TFAM, PPARGC1A, PPA1, PPP2R4, SLC2A4, MAPK1, PRKACA, α1A, α1B, SCN4B, KCNE1.

According to another aspect, there is provided a method of producing a multichambered cardiac organoid comprising at least two chambers beating in synchrony, the method comprising coculturing a mass of cardiomyocytes and endothelial cells in a geometrically confined culture space such that anisotropic stress gradients are generated in the cell mass, thereby producing a multichambered cardiac organoid.

According to some embodiments, the method comprises culturing about 6.8×10\hat{ }4 cells in a microwell comprising a diameter of between 1-1.2 mm.

According to some embodiments, the coculture comprises a ratio of cardiomyocytes to endothelial cells of between 1.5:1 and 2.5:1.

According to another aspect, there is provided a multichambered cardiac organoid comprising at least two chambers beating in synchrony produced by a method of the invention.

According to another aspect, there is provide a method of evaluating cardiac cell function, the method comprising exposing a multichambered cardiac organoid of the invention to a condition and measuring at least one parameter of the multichambered cardiac organoid.

According to some embodiments, the condition is selected from: application of a drug or chemical, hypoxic conditions, circulation conditions, change in metabolite exposure, change in hormone exposure, and genetic mutation of cells in the organoid.

According to some embodiments, the at least one parameter is electro-mitochondrial synchronization.

According to another aspect, there is provided a sensing system comprising:
an illumination source;
a first photomultiplier tube (PMT) sensor;
a second PMT sensor and
a controller configured to:
   control the illumination source to illuminate a microparticle embedded in a tissue or cell aggregate with a photon beam having a first wavelength;
   detect, by the first PMT sensor, a first signal indicative of photons reflected from the microparticle at the first wavelength;
   detect, by the second PMT sensor, a second signal indicative of emission from the microparticles at a second wavelength, wherein the microparticles comprise an excitable molecule quenchable by a cofactor
   measure a shift between a frequency of the first signal and a frequency of the photon beam, determine background noise based on the measured shift and reduce background noise from the second signal; and
   calculate temporal cofactor consumption of the tissue or cell aggregate based on the background noise-reduced second signal.

According to some embodiments, the temporal cofactor consumption is indicative of the oxygen level in the tissue or cell aggregate.

According to some embodiments, the controller is further configured to detect a change in intensity of the first signal and calculate relative displacement of the microparticle, based on the detected change.

According to some embodiments, the detected changes in the intensity of the signal is indicative of the relative displacement of the microparticle, optionally wherein the displacement is measured in an axis perpendicular to the photons beam.

According to some embodiments, the controller is further configured to sense field potential of the tissue or cell aggregate from an array of microelectrodes for measuring the electrical activity of the tissue or cell aggregate simultaneously to detecting the first signal and the second signal.

According to another aspect, there is provided a method of evaluating cellular function, the method comprising:
a. placing tissue, an organoid or a cellular aggregate in a sensing system of the invention,
b. applying a condition to the tissue, organoid or cellular aggregate; and
c. measuring at least cofactor consumption in the tissue, organoid or cellular aggregate,
thereby evaluating cellular function.

According to some embodiments, the sensing system is a sensing system comprising a controller further configured to sense field potential of the tissue or cell aggregate from an array of microelectrodes for measuring the electrical activity of the tissue or cell aggregate simultaneously to detecting the first signal and the second signal and the measuring comprises measuring cofactor consumption, displacement and electrical field potential in the tissue, organoid or cellular aggregate and wherein a significant deviation in displacement, cofactor consumption, and electrical field potential after applying the condition as compared to displacement, cofactor consumption, and electrical field potential before applying the condition or as compared to control untreated tissue, organoid or cellular aggregate is indicative of electro-mitochondrial desynchronization.

According to some embodiments, a cardiac or brain organoid is placed in the sensing system.

According to some embodiments, the applying a condition is selected from: application of a drug or chemical, application of hypoxic conditions, application of circulation conditions, changing metabolite exposure, changing hormone exposure, and genetic mutation of cells in the tissue, organoid or aggregate.

According to another aspect, there is provided a method of selecting a subject suffering from a disease or disorder suitable for treatment with an agent capable of
a. modulating mitochondrial calcium concentration in a tissue of the disease or disorder in the subject;
b. modulating mitochondrial calcium channel activity in the tissue; or
c. a combination there thereof;
   the method comprising determining the presence of electro-mitochondrial desynchronization in the subject, wherein the presence of the desynchronization indicates the subject is suitable for treatment.

According to some embodiments, the determining comprises at least one of:
a. determining mitochondrial calcium concentration in a sample obtained from the subject and wherein a concentration beyond a predetermined threshold indicates desynchronization, optionally wherein the predetermined threshold is calcium concentration in a healthy subject or in a subject suffering from the disease or disorder not characterized by electro-mitochondrial desynchronization;
b. observing in the patient arrhythmic or proarrhythmic symptoms that do not respond to antiarrhythmic treatment that targets electrical activity through membrane channels thus indicating desynchronization, optionally wherein the antiarrhythmic treatments are selected from: sodium channel blockers, beta blockers, potassium channel blockers, nondihydropyridine calcium channel blockers, adenosine and digoxin;
c. EKG, EEG or EMG, wherein an abnormal readout indicates desynchronization, optionally wherein an abnormal readout comprises at least one of late potentials, reduced R waves, and increased R/R ratios;
d. confirming exposure to an agent that is known to produce electro-mitochondrial desynchronization, optionally wherein the agent is an agent selected from those provided in Table 1 and toluene, trichloroethane, xylene, heptanes, hexane, ethyl ether trichloroethylene, trichlorotrifluoroethane, carbon monoxide, carbon disulfide, pesticides, methane-derived halogenated hydrocarbons, organic nitrates, arsenic, cadmium, cobalt, organic solvents, and metals; and
e. confirming a medical history containing diagnosis of symptoms that are indicative of electro-mitochondrial desynchronization.

According to another aspect, there is provided a multichambered cardiac organoid comprising cardiomyocytes and endothelial cells in a geometrically confined compartment, wherein the chambers comprise chamber walls capable of beating.

According to another aspect, there is provided a method of testing a drug comprising contacting the multichambered cardiac organoid of the invention with the drug.

According to another aspect, there is provided a method of treating a disease or disorder characterized by electro-mitochondrial desynchronization in a subject in need thereof, the method comprising administering to the subject a therapeutically effective amount of an agent capable of
a. increasing mitochondrial calcium concentration in a tissue of the disease or disorder in the subject;
b. increasing mitochondrial calcium channel activity in the tissue; or
c. a combination there thereof;
thereby treating a disease or disorder characterized by electro-mitochondrial desynchronization.

According to another aspect, there is provided a method of treating a disease in a subject in need thereof with a first agent that produces a cardiac side effect, the method comprising administering:
a. the first agent; and
b. a second agent that: increases mitochondrial calcium concentration in a heart tissue, increases mitochondrial calcium channel activity in a heart tissue, or both;
   thereby treating the disease in the subject.

According to another aspect, there is provided a method of producing a cardiac organoid, the method comprising coculturing cardiomyocytes and endothelial cells in conditions such that the cells are geometrically confined, thereby producing a cardiac organoid.

According to another aspect, there is provided a method of measuring properties of a tissue or cell aggregates, comprising:
illuminating microparticle embedded in tissue or cell aggregate with photons beam having a first wavelength; detecting, by a first sensor, a first signal indicative of photons reflected from the microparticle at the first wavelength;
detecting a change in intensity of the first signal; and
calculating relative displacement of the microparticle, based on the detected change.

According to another aspect, there is provided a sensing system comprising:
an illumination source;
a first photomultiplier tube (PMT) sensor; and
a controller configured to:
   control the illumination source to illuminate microparticle embedded in tissue or cell aggregate with photons beam having a first wavelength; detecting, by a the first PMT sensor, a first signal indicative of photons reflected from the microparticle at the first wavelength;
   detect a change in intensity of the first signal; and
   calculate relative displacement of the microparticle, based on the detected change.

According to another aspect, there is provided a method of testing a therapeutic agent for cardiac side effects, the method comprising:
a. placing a cardiac organoid within a sensing system of the invention;
b. adding the therapeutic agent to the cardiac organoid; and
c. measuring displacement, cofactor consumption, and electrical field potential in the cardiac organoid;
wherein a significant deviation in any one of displacement, cofactor consumption, and electrical field potential after adding the therapeutic agent as compared to displacement, cofactor consumption, and electrical field potential before adding the therapeutic agent or in a control untreated cardiac organoid is indicative of a cardiac side effect caused by the therapeutic agent;
   thereby testing a therapeutic agent for cardiac side effects.

According to some embodiments, the endothelial cells are microvascular endothelial cells.

According to some embodiments, the cardiomyocytes are derived or produced from induced pluripotent stem cells.

According to some embodiments, the multichambered cardiac organoid further comprises vascular structures.

According to some embodiments, the multichambered cardiac organoid comprises at least 2 chambers.

According to some embodiments, the multichambered cardiac organoid comprises circumferentially aligned cardiomyocytes surrounding hollow chambers.

According to some embodiments, the multichambered cardiac organoid comprises elongated cardiomyocytes organized in a sarcomeric pattern.

According to some embodiments, the multichambered cardiac organoid comprises capillaries within a wall of the chambers.

According to some embodiments, the multichambered cardiac organoid comprises cardiac fibroblast-like cells.

According to some embodiments, the fibroblast-like cells are POSTN positive cells.

According to some embodiments, the multichambered cardiac organoid comprises pacemaker-like cell clusters.

According to some embodiments, the pacemaker-like cell clusters are HCN4 and SHOX2 positive.

According to some embodiments, the multichambered cardiac organoid comprises an outer epicardium.

According to some embodiments, the epicardium comprises cells positive for WT1 and TBX18.

According to some embodiments, the chamber walls comprise PECAM-1 positive endocardial-like cells.

According to some embodiments, the multichambered cardiac organoid is capable of synchronized beating.

According to some embodiments, the synchronized beating persists for at least 1 week in culture.

According to some embodiments, the beating is at least 50 beats per minute (bpm).

According to some embodiments, the multichambered cardiac organoid comprises increased expression of at least one factor selected from KCNJ2, KCNJ8, TMNI3, MYH7, AKAP6, PPKAA2, PGC1A, RAR2, CASQ2, and CAV3, as compared to isolated cardiomyocytes and/or fetal cardiac tissue.

According to some embodiments, the multichambered cardiac organoid comprises increased expression of 2-10 genes selected from the group consisting of: KCNJ2, KCNJ8, TMNI3, MYH7, AKAP6, PPKAA2, PGC1A, RAR2, CASQ2, and CAV3, as compared to isolated cardiomyocytes and/or fetal cardiac tissue.

According to some embodiments, the multichambered cardiac organoid comprises at least one of increased basal respiration, oxidative phosphorylation or mitochondrial maximal capacity as compared to cardiomyocytes in culture.

According to some embodiments, the multichambered cardiac organoid is capable of producing a physiological response to a therapeutic agent.

According to some embodiments, the therapeutic agent is epinephrine.

According to some embodiments, the therapeutic agent is amiodarone.

According to some embodiments, the method further comprises testing a physiological output of the multichambered cardiac organoid after the contacting.

According to some embodiments, the method further comprises comparing the physiological output to an output measured before the contacting.

According to some embodiments, the testing is testing for a negative cardiac side effect.

According to some embodiments, the side effect is arrythmia.

According to some embodiments, the drug is an anti-cancer agent.

According to some embodiments, the drug is a calcium signaling targeting agent.

According to some embodiments, the drug is a calcium channel blocker.

According to some embodiments, the method further comprises inducing a cardiac deficiency, condition or disease in the multichambered cardiac organoid before the contacting and wherein the drug is a therapeutic designed to treat the deficiency, condition or disease.

According to some embodiments, the condition is arrythmia.

According to some embodiments, the increasing mitochondrial calcium or increasing mitochondrial calcium chancel activity comprises increasing mitochondrial activity.

According to some embodiments, the increasing mitochondrial calcium channel activity comprises increasing mitochondrial calcium uniporter (MCU) activity.

According to some embodiments, the agent that increases MCU activity is an agent that blocks interaction of a drug with MCU, wherein the drug inhibits MCU activity.

According to some embodiments, the administered agent is an MCU activator.

According to some embodiments, the MCU activator is metformin.

According to some embodiments, the disease or disorder is selected from: arrhythmia, cardiomyopathy, seizures, epilepsy, motor neuron spasms, muscle weakness, and muscular atrophy, optionally wherein the arrythmia is a cancer treatment induced arrythmia (CTIA).

According to some embodiments, the first agent is a calcium signaling targeting agent.

According to some embodiments, the first agent is a calcium channel blocker.

According to some embodiments, the disease is cancer and the first agent is an antineoplastic agent.

According to some embodiments, the disease is an inflammatory disease and the first agent is an anti-inflammatory agent.

According to some embodiments, the disease is a disease of the central nervous system (CNS) and the first agent is a CNS agent.

According to some embodiments, the disease is a gastrointestinal disease and the first agent is a gastrointestinal agent.

According to some embodiments, the disease is genital or urinary disease and the first agent is a genitourinary agent.

According to some embodiments, the disease is an allergic reaction and the first agent is an antiallergic agent.

According to some embodiments, the disease is an infection and the first agent is an anti-infective agent.

According to some embodiments, the disease is a cardiovascular disease and the first agent is a cardiovascular agent.

According to some embodiments, the first agent is selected from the agents provided in Table 1.

According to some embodiments, the increasing mitochondrial calcium channel activity comprises increasing MCU activity.

According to some embodiments, the second agent is metformin.

According to some embodiments, the subject does not suffer from a metabolic disorder, is not treated for a metabolic syndrome, or both.

According to some embodiments, the metabolic disorder is diabetes or hyperglycemia.

According to some embodiments, the condition is a concentration of about 6.8×10\hat{ }4 cells in a microwell of diameter between 1-1.2 mm.

According to some embodiments, the coculture comprises a ratio of cardiomyocytes to endothelial cells of between 1.5:1 to 2.5:1.

According to some embodiments, the endothelial cells are microvascular cardiac endothelial cells.

According to some embodiments, the culture comprises the addition of vascular endothelial growth factor (VEGF).

According to some embodiments, the culturing is for a time sufficient for the formation of multiple hollow chambers surrounded by cardiomyocytes and synchronized beating.

According to some embodiments, the method is a method of producing a cardiac organoid of the invention.

According to some embodiments, the culturing is for a time sufficient for formation of an organoid characterized by characteristics of the cardiac organoid of the invention.

According to some embodiments, the detected change in the intensity of the signal is proportional of the relative displacement of microparticle.

According to some embodiments, the displacement is measured in an axis perpendicular to the photons beam.

According to some embodiments, the method further comprises:
detecting, by a second sensor, a second signal indicative of emission from microparticles embedded in the tissue or cell aggregate at a second wavelength, wherein the microparticles comprise an excitable molecule quenchable by a cofactor;
calculating temporal cofactor consumption of the tissue or cell aggregate based on the first and second signals.

According to some embodiments, the temporal cofactor consumption is correlated to a difference in frequencies of the first signal and the second signal.

According to some embodiments, the temporal cofactor consumption is the oxygen level of the tissue or the cell aggregate.

According to some embodiments, the method further comprises filtering the second signal using parameters of the photons beam.

According to some embodiments, filtering comprises:
measuring a shift between a frequency of the second signal and a frequency of the photons beam;
determining background noise based on the measure shift; and
reducing the background noise form the second signal.

According to some embodiments, the method further comprises:
sensing field potential of the tissue or cell aggregates from an array of microelectrodes for measuring the electrical activity of the tissue or cell aggregates at the same time as the optical measurement.

According to some embodiments, the method further comprises comparing frequencies of the first signal, the second signal and the field potential of the tissue.

According to some embodiments, if the comparison between the frequencies yields a deviation lower than a threshold, the comparison is indicative of a healthy tissue or cell aggregates.

According to some embodiments, the detected changes in the intensity of the signal is indicative of the relative displacement of microparticle.

According to some embodiments, the displacement is measured in an axis perpendicular to the photons beam.

According to some embodiments, the system further comprises:
a second PMT sensor,
and wherein the controller is further configured to:
   detect, from a second PMT sensor, a second signal indicative of emission from microparticles embedded in the tissue or cell aggregate at a second wavelength, wherein the microparticles comprise an excitable molecule quenchable by a cofactor;
   calculate temporal cofactor consumption of the tissue or cell aggregate based on the first and second signals.

According to some embodiments, the temporal cofactor consumption is correlated to a difference in frequencies of the first signal and the second signal.

According to some embodiments, the temporal cofactor consumption is the oxygen level of the tissue or the cell aggregate.

According to some embodiments, the controller is further configured to filter the second signal using parameters of the photons beam.

According to some embodiments, filtering comprises:
measuring a shift between a frequency of the second signal and a frequency of the photons beam;
determining background noise based on the measure shift; and
reducing the background noise form the second signal.

According to some embodiments, the controller is further configured to:
sense field potential of the tissue or cell aggregates from an array of microelectrodes for measuring the electrical activity of the tissue or cell aggregates simultaneously to detecting the first signal.

According to some embodiments, the controller is further configured to:
compare frequencies of the first signal, the second signal and the field potential of the tissue.

According to some embodiments, if the comparison between the frequencies yields a deviation lower than a threshold, the comparison is indicative of a healthy tissue or cell aggregates.

According to some embodiments, the cardiac organoid is a cardiac organoid capable of beating.

According to some embodiments, the cardiac organoid is a multichambered cardiac organoid of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1O include illustrations, graphs, micrographs and heatmaps showing the production of human-induced pluripotent stem cells (hiPSC)-derived vascularized cardiac organoids. (1A) Scheme describing the formation of hiPSC-derived vascularized cardiac organoids. Cardiomyocytes are differentiated from hiPSC over a 10-day period, dissociated, and mixed with microvascular endothelial cells in a 3D scaffold. Cells form beating organoids in 4-days within the well, developing multiple chambers under anisotropic stress over 25 days. (1B) Representative time-lapse sequence of brightfield images depicting the formation of oxygen sensors-embedded vascularized cardiac organoids. A single mass forms on day 4, beats by day 10, and acquires a smooth exterior and uniform-synchronized beating by day 25. Bar=250 μm. (1C) Left: Finite element model of the von Mises stress distribution in 3D cardiac organoids formed on a solid surface (free organoid), geometrically confined in a microwell, or vascularized and confined in a microwell. (1D) Cross-section stress profile and DNA staining showing homogenous stress of free organoid resulting in cardiac spheroid, radial stress gradients of geometrically confined organoid resulting in single-chamber formation. Anisotropic stress distribution of vascularized organoids resulting in multi-chambered cardiac organoid formation. Bar=200 μm. (1E) Confocal cross-sections and expression distribution of mechanical stress markers Lamin A/C (Lamin) and YAP1 in cardiac organoids. Free organoids show a homogenous distribution of both stress markers, while geometrically confined ones show the predicted circumferential stress. Anisotropic stress distribution in vascularized organoids results in a multi-chambered cardiac organoid formation. Bar=100 μm. (1F) Confocal cross-section of vascularized cardiac organoid showing circumferentially aligned cardiac fibers (α-Actinin) surrounding vertical-like chambers, embedded with a vascular network of cardiac endothelial cells (CECs). Bar=50 μm. Confocal section of the organoid wall showing aligned cardiac fibers (cTnT) pierced with a patent microvasculature capillary (CEC). Bar=50 μm. Scanning electron micrograph of vascularized cardiac organoid demonstrates lumen formation. Bar=10 μm. (1G) Immunofluorescence of cardiac troponin T (cTnT) and α-actinin fibers. The fibers form long sarcomeres aligned along with the cardiac tissue; a phenotype associated with mature cardiac tissue. Bar=10 μm. (1H) Confocal cross-section of vascularized cardiac organoid showing myocardial-like layer is weaved with POSTN cardiac fibroblast-like cells and isolated cell clusters expressing the pacemaker-associated markers HCN4 and SHOX2. Bar=100 μm. (1I) Confocal cross-section of vascularized cardiac organoid showing circumferentially oriented epicardium cells on the cardiac organoid surface, express the transcriptional regulator Wilm's Tumor Gene 1 (WT1) and TBX18 embedded with a vascular network of cardiac endothelial cells (CECs). Bar=100 μm. (1J) Confocal cross-section further shows that the organoid's cavities are lined with PECAM-1 endocardial-like cells. Bar=100 μm. (1K) Clustering of RNA-Seq data from iPS-derived cardiomyocytes and fetal cardiomyocytes compared to vascularized cardiac organoids and adult cardiomyocytes, RNA sequencing of the cardiac organoids, showing expression signatures associated with endocardium, epicardium, cardiac fibroblast cells, and pacemaker cells. (1L) Representative time-lapse sequence of confocal images depicting the formation of vascular network apparent. Confocal microscopy shows the distribution of GFP-expressing cardiac endothelial cells (CECs) in the organoid. Vascular network apparent by day 10. (1M) Confocal cross-sections of vascularized cardiac organoids reveal circumferentially aligned cardiac fibers (α-Actinin) surrounding vertical-like chambers, embedded with a vascular network of cardiac endothelial cells. (1N) Quantification of chamber formation in the three stress regimes. Multiple chambers occur 87% of the time under geometric confinement and vasculature. (1O) Confocal cross-section of vascularized cardiac organoid stained for TUNEL to indicate apoptosis, and Carbonic Anhydrase ix (CA-IX) expression which is considered as an endogenous biomarker of hypoxia to determine the upregulated hypoxic regions. Immunofluorescent staining demonstrated slight expression in TUNEL and CA-IX. Bar=100 μm.

FIGS. 2A-2F include heatmaps, graphs, a micrograph, a table, and diagrams showing the functional characterization of human cardiac organoids. (2A) Transcriptomic analysis of iPS-derived cardiomyocytes and fetal cardiomyocytes compared to vascularized cardiac organoids and adult cardiomyocytes. (2B) Principal component analysis (PCA) of gene expression patterns of genes between the cardiac organoids and adult cardiomyocytes show that the clusters 2 and 3 that distinguished well between adult and fetal cardiac tissues clustered the cardiac organoids with the adult tissue (2C) Vascularized cardiac organoids show a spontaneous beating of 66±5 beats per minute (bpm). Organoids retain synchronized beating under pharmaceutical stimulation, resulting in a physiological-like response to drugs. Stimulation with 100 μM epinephrine increases contraction rate to 88±7 bpm and relative contraction by 18%, while stimulation with 10 μM amiodarone decreased the rate to 52±4 bpm and contraction by 28%. (2D) Seahorse MitoStress test of cardiac organoids compared to hiPSC-derived cardiomyocytes. Reconstructing the cardiomyocytes into vascularized cardiac organoids increased the oxidative phosphorylation by 85%, as well as maximal respiratory capacity by 58% (n=3, p<0.05), while glycolysis was unaffected. (2E) Confocal cross-section of vascularized cardiac organoid embedded with oxygen phosphorescence sensors following 14 days in culture. (2F) Intracellular metabolic fluxes of vascularized cardiac organoids during the cardiac cycle. Glucose utilization and calculated ATP production are shown as nmol/min/106 cells. p<0.05, **p<0.01, **p<0.001. Error bars represent ±Standard error of the mean (S.E.). Significance was determined using a one-tailed heteroscedastic student-test. Bar=250 μm.

FIGS. 3A-3L include illustrations, images, micrographs, and graphs showing that integrated opto-electrical sensors permit real-time simultaneous measurement of cardiac metabolism, contraction, and action potential. (3A) Schematic of an integrated metabolic-electrical-mechanical sensor chip. Lifetime-based Ru-CPOx phosphorescence sensors are embedded in cardiac organoids and probed by two frequency LED modulation. Oxygen is measured as a phase shift in the emission signal (PMT), while a second detector is used for noise reduction and measurement of tissue contraction (cPMT). Electrophysiological activity is recorded using a nanofabricated gold microelectrode array (MEA). All measurements are synchronized and processed in real-time by a single microprocessor. (3B) Optical measurement schematics. Oxygen concentration (left) is measured by the phosphorescence quenching of the signal by ambient triplet oxygen, decreasing decay time, resulting in a shorter phase shift. contraction rate (right) is measured by monitoring the sub-second temporal change of the average phosphorescent intensity which is correlated to cardiac contraction. (3C) Exploded view and image of the sensor integrated heart-on-a-chip platform. The device is composed of a 3D printed casing supporting a PDMS microwell grid, layered on a nanofabricated gold-on-glass MEA. The device is sealed by a 3D printed holder containing connectors to the MEA leads. MEA-integrated chip containing 45 gold electrodes on a 25×25 mm glass chip. A PDMS microwell array supports 9 cardiac organoid-carrying scaffolds; each containing five recording electrodes. (3D) Representative image of oxygen sensors embedded in a cardiac organoid formed within a MEA integrated scaffold. The IEA's gold electrodes are seen as shaded areas. Bar=200 μm. (3E) Scanning electron micrograph displaying a 30-day old cardiac organoid formed in an integrated scaffold. Bar=200 μm. (3F-H) Simultaneous measurements and Fast Fourier transform (FFT) analysis of (3F) contraction, (3G) field potential, and (3H) interstitial oxygen in cardiac organoids during spontaneous beating (see FIG. 9A). Interstitial oxygen concentration shows oscillatory behavior during the cardiac cycle, yielding distinct single frequency peaks in FFT analysis correlated to the mechanical and electrical behavior of the cardiac tissue. (3I-J) Representative graphs of the cardiac organoid's (3I) contraction and (3J) interstitial oxygen behaviors following stimulation with 100 μM epinephrine. Epinephrine increases the contractility and contraction rate of the vascularized cardiac organoids over time while decreasing the mean interstitial oxygen concentration in the organoid (dotted line) and increasing its oscillation frequency. The analysis demonstrates that the contractile behavior is correlated to the oxygen behavior (see FIG. 9A-9D). (3K) Representative graphs of the cardiac organoid's contraction, field potential, and interstitial oxygen behaviors and Fast Fourier transforms (FFT) following treatment with 10 μM of myosin II inhibitor Blebbistatin (see FIG. 9B). Blebbistatin inhibits the contraction of vascularized cardiac organoids but does not affect the FPs or interstitial oxygen oscillations frequency and intensity, suggesting there is an electro-mitochondrial coupling that does not involve actomyosin activity. (3L) Representative graphs of the cardiac organoid's contraction, field potential, and interstitial oxygen behaviors following treatment with 25 μM of Naᵥ channel inhibitor Tetrodotoxin (TTX). Exposure to TTX resulted in a complete loss of field potential generation and associated mechanical contraction. Oxygen oscillation decayed simultaneously with the field potential.

FIGS. 4A-4G include micrographs and graphs showing disruption of electro-mitochondrial coupling induces arrhythmic behavior. (4A) Immunofluorescent TMRE staining for mitochondrial membrane potential (MMP) and corresponding frequency heatmap in 2D-cultured hiPSC-derived cardiomyocytes. Contraction rate and corresponding mitochondrial membrane potential oscillations captured by live fluorescence microscopy (see Materials and methods). The mitochondrial membrane potential of beating 2D hiPSC-derived cardiomyocytes oscillated in the frequency of contraction, while neighboring non-beating cells did not oscillate (n=5). Bar=25 μm. (4B) Kinetic measurements of dynamic changes in cell contraction and mitochondrial calcium [Ca²⁺]ₘ following treatment with 10 μM mitochondrial calcium uniporter (MCU) inhibitor KB-R7943. [Ca²⁺]ₘ was measured using Rhod-2AM under stable mitochondrial dye staining (MitoTracker). Contraction was measured by visual analysis (see Materials and methods). Acute MCU inhibition dramatically decreases cardiomyocyte contractility, and [Ca²⁺]ₘ oscillation amplitude without affecting contraction rate. (4C) Phase image of hiPSC-derived vascularized cardiac organoid in an integrated metabolic-electro-mechanical sensor chip (n=4). Bar=100 μm. (4D) Real-time measurements of interstitial oxygen content and intracellular metabolic fluxes of vascularized cardiac organoids exposed to 10 μM MCU inhibitor KB-R7943. Fluxes were calculated at 0-, 15-, and 25-minutes following exposure to 10 μM KB-R7943. Glucose utilization and calculated ATP production are shown as nmol/min/10⁶ cells (see Materials and methods). Interstitial oxygen content suggests a progressive decrease in the oxygen uptake of the cardiac organoid caused by MCU inhibition, resulting in a decrease in ATP production and a growing ATP gap. (4E) Representative kinetic measurements of contraction, field potential, and interstitial oxygen content in cardiac organoids exposed to 10 μM KB-R7943 (n=9). (4F) Kinetic simultaneous measurements of contraction frequency, oxygen oscillation frequency, and field potential oscillation frequency after MCU inhibition. MCU inhibition resulted in a correlated increase in all oscillation rates. In contrast to oscillation rates, MCU inhibition progressively decreases the magnitude of cardiomyocyte contraction, electrical activity, and oxygen uptake, and led to arrhythmogenic cardiac behavior (n=9). (4G) Representative changes in single contraction measurements of contraction, field potential, and interstitial oxygen content due to treatment with 10 μM KB-R7943. MCU inhibition caused uncoupling between mitochondrial and electro-mechanical activity, resulting in arrhythmic cardiac behavior.

FIGS. 5A-5E include a micrograph, graphs, diagrams, and illustrations showing CRISPR/Cas9 knockout of MCU disrupts electro-mitochondrial coupling and induces arrhythmic behaviour. (5A) Kinetic Rhod-2AM measurements of mitochondrial membrane potential (MMP) in beating 2D-cultured hiPSC-derived cardiomyocytes. Non-targeting sgRNA had no effect on [Ca²⁺]ₘ showing a dominant frequency of 0.8 Hz, while MCU knockout (MCU^{KO}) showed a 50% decrease in [Ca²⁺]ₘ and oscillation magnitude while increasing oscillation rate to 1.3 Hz (n=7, p<0.01). (5B) Immunofluorescent staining of MCU in non-homogenous MCU^{KO} chimeric vascularized cardiac organoid. Immunofluorescent staining demonstrated a marked reduction in MCU expression. Bar=100 μm. (5C-5D) (5C) Representative kinetic measurements and (5D) dynamic analysis of contraction, field potential, and interstitial oxygen content in non-homogenous MCU^{KO} or sgRNA chimeric cardiac organoids (n=9, p<0.001). Disruption of MCU expression in the organoid reduced organoid contractility, reduced oxygen oscillation magnitude, and showed clear arrhythmogenic behavior in organoid field potential. (5E) Scheme depicting the changes in the interplay between the electrical, mitochondrial, and mechanical activity in cardiomyocytes due to MCU inhibition. The electro-mitochondrial coupling is underlying this coordination, and its uncoupling by MCU inhibition results in uncoordinated activity and arrhythmogenic behaviour. *p<0.05, **p<0.01, ***p<0.001. Error bars represent ±S.E. Significance was determined using a two-tailed heteroscedastic student-test.

FIGS. 6A-6F include a table, illustrations, and graphs showing that mitoxantrone inhibition of electro-mitochondrial coupling and resultant arrhythmia is partly reversed by metformin. (6A) Structure, clinical indications, and maximal physiological concentration (Cmax) of Mitoxantrone and Metformin. (6B) Scheme depicting the electro-mitochondrial coupling disruption in cardiomyocytes exposed to mitoxantrone, and the recovery resulting from an addition of metformin. MCU inhibition by Mitoxantrone causes an insufficient ATP production upon depolarization resulting in arrhythmogenic behavior. The addition of metformin improves calcium entry to mitochondria during depolarization and allows more ATP to be produced. (6C-6D) (6C) Representative recording and (6D) mean mitochondrial calcium, measured using live imaging of Rhod-2AM dye in different hiPSC-derived cardiomyocytes cultures treated with 10 μM MCU inhibitor mitoxantrone or DMSO (control), followed by treatment with 10 μM mitoxantrone (mitoxantrone) or 10 μM mitoxantrone and 100 μM AMP-activated protein kinase activator metformin (mitoxantrone+metformin; n=4). Mitoxantrone alone resulted in a 79% decrease in mitochondrial calcium (p<0.001). The addition of 100 μM metformin to the mitoxantrone treatment resulted in a 3.4-fold increase in mean mitochondrial calcium content (p<0.001). (6E) Representative simultaneous kinetic measurements of contraction, field potential, and interstitial oxygen content in cardiac organoids exposed to DMSO (control), 10 μM mitoxantrone (mitoxantrone), or 10 μM mitoxantrone and 100 μM metformin (mitoxantrone+metformin). Mitoxantrone caused irregular cardiac contraction and field potential. Concurrent treatment with metformin reverts the effect, displaying regular heart contraction and field potential. (6F) Representative changes in single contraction measurements of contraction, field potential, and interstitial oxygen content due to treatment with 10 μM mitoxantrone or concurrent treatment with 10 μM mitoxantrone and 100 μM metformin. MCU inhibition by mitoxantrone caused electro-mitochondrial uncoupling, resulting in arrhythmic cardiac behavior. Concurrent treatment with Metformin and Mitoxantrone rescues the arrhythmic cardiac behavior, displaying coordinated cardiac behavior. *p<0.05, **p<0.01, ***p<0.001. Error bars represent ±S.E. Significance was determined using a two-tailed heteroscedastic student-test.

FIGS. 7A-7C include showing diagrams and micrographs showing generation of multi-chambered vascularized cardiac organoids. Finite element model of (7A) the von Mises stress distribution and (7B) Gaussian displacement in 3D cardiac organoids formed on a solid surface (free organoid), geometrically confined in a microwell, or vascularized and confined in a microwell. The finite element model shows a gradient of mechanical stress contributes to the formation of cardiac chambers. (7C) Confocal cross-sections of organoids stained for mechanical stress markers Lamin A/C (Lamin) and YAP1. Free organoids show a homogenous distribution of both stress markers, while geometrically confined ones show the predicted circumferential stress. Anisotropic stress distribution in vascularized organoids results in a multi-chambered cardiac organoid formation. Bar=100 μm.

FIGS. 8A-8D include illustrations and a graph showing establishment of an integrated 2-PMT Heart-on-a-chip platform. (8A) Scheme and typical measurements depicting the advantages of using a 2-PMT system over a single PMT system. The addition of the second detector (cPMT), which measures the excitation signal, reduces the noise and enables accurate measurements at sub-second resolution. The second PMT also allows an emission-independent measurement of tissue contraction (see Materials and methods). (8B) calibration measurements of the reflected signal measured by the second PMT in different displacements. Curve fitting reveals a sigmoidal relationship between the emission intensity, measured by peak-to-peak voltage (V_{P-P}), and the sensor displacement. Cardiac displacement was measured by the embedded oxygen beads inside the cardiac organoids during a contraction when the beads move at different distances from the focal point. The Sigmoidal fit shows a correlation of R-square: 0.9835 and RMSE below 4. (8C) Schematic depicting the fabrication process of the MEAs using lift-off lithography technique. computer-aided design Solidworks® was used to design the MEAs. Cleaned microscope slides (Corning@) were used as the substrate. Layers of LOR 5b and AZ1505/positive photoresists were spin-coated on the substrates. Direct laser writer (Microtech®, LW405-A) was used after the pattern was drawn in CleWins® layout. After developing by AZ 726 MIF, Sputter deposition of Titanium and Gold were performed, respectively. The fabrication process was completed by lifting off the photoresist in Acetone. (8D) PDMS micro scaffolds mounted on the MEA transparent chip supporting the formation of 9 cardiac organoids. PDMS scaffolds were fabricated using a laser cutting CNC machine and covalently bound on top of the MEAs chip using oxygen plasma activation. FIG. 8A is an illustration of a prior art system and a system according to some embodiments of the invention. Schematic dissipating the advantages of using a 2-PMT system. Oxygen is measured by the phase shift between the emission signal detected by the photomultiplier (PMT), and the excitation signal. Thus, adding a second detector (cPMT), that measures the excitation signal, reduce the noise to enable accurate measurements at sub-second resolution. The second PMT also allows the emission-independent measurement of tissue contraction. (8B) Sigmoidal curve fitting examines the relationship between the emission intensity measured in peak-to-peak voltage (VP-P) from the oxygen bead and its displacement using the 2-PMT systems. Cardiac displacement was measured by the embedded oxygen beads inside the cardiac organoids during a contraction when the beads move at different distances from the focal point. The Sigmoidal fit shows a correlation of R-square: 0.9835 and RMSE below 4.

FIG. 8E is a flowchart of a method of measuring properties of a tissue or cell aggregates according to some embodiments of the invention.

FIGS. 9A-9D include non-limiting schemes and graphs showing real-time metabolic measurement of vascularized cardiac organoids. (9A) Simultaneous measurements of contraction, field potential, and interstitial oxygen in cardiac organoids during spontaneous beating. Interstitial oxygen concentration shows oscillatory behavior during the cardiac cycle, yielding distinct single frequency peaks in FFT analysis (9B) correlated to the mechanical and electrical behavior of the cardiac tissue. (9C) Schematic of the electrophysiology recording system connected to the MEA to track the spontaneous cardiac field potentials (FPs) simultaneously in real-time. Field potentials were amplified and filtered using an integrated signal conditioning circuit (AD8232), with a 2-pole adjustable high-pass filter, 3-pole adjustable low-pass filter, adjustable gain, and medical instrumentation amplifiers. Arduino MEGA 2560® microcontroller was used as an analog-to-digital converter (ADC). (9D) representative raw and filtered field potential measurements of cardiac organoids. A finite impulse response filter was applied in real-time using a custom-written MATLAB code (methods). Periodically, an anti-aliasing filter was used to eliminate noise with a frequency higher than 8 Hz, created by the electromagnetic field of the different instruments in the workspace.

FIGS. 10A-10G include graphs showing real-time metabolic measurement of vascularize cardiac organoids under epinephrine stimulation. (10A) Analysis of the kinetic behavior of the organoid's contraction rate during prolonged stimulation by 100 μM epinephrine. Kinetic analysis suggests that epinephrine stimulation results in a sigmoidal-like change in the organoid contraction rate. (10B-10C) Representative relation graphs between (10B) contraction amplitude (contractility)—contraction rate, and (10C) interstitial oxygen content—contraction rate during prolonged epinephrine stimulation. Analysis suggests a correlation between an increase in cardiac organoid contractility and oxygen consumption. (10D) Representative frequency histograms of interstitial oxygen measurements at 0, 15, and 90 minutes after stimulation with 100 μM epinephrine. Analysis shows that an increase in oxygen consumption correlates to an increase in interstitial oxygen content variability correlative to the increased oxygen amplitudes measured. (10E-10F) Representative correlation analysis between (10E) oxygen oscillation frequency and contraction frequency and (10F) oxygen oscillation amplitude and contractility reveal a direct linear correlation between the oscillatory behavior of the interstitial oxygen and organoid contraction. (10G) Representative graphs of Fast Fourier transforms (FFT) analysis showing the cardiac organoid's contraction, field potential, and interstitial oxygen frequency behaviors following treatment with 10 μM of myosin II inhibitor Blebbistatin. Blebbistatin treatment blocked cardiac contraction, and sensor movement but did not affect the field potential or the oxygen oscillation frequency and intensity.

FIGS. 11A-11L include micrographs, graphs, and heatmaps showing live mitochondrial imaging reveals oscillations in mitochondrial membrane potential. (11A) Immunofluorescent micrograph of mitochondrial membrane potential (ΔΨm) measured using live imaging of TMRE dye. (11B) Kinetic analysis of mitochondrial membrane potential (ΔΨm) using TMRE. The mitochondrial membrane potential of hiPSC-derived cardiomyocytes oscillates in the frequency of contraction. The mitochondrial membrane potential of non-beating cells did not oscillate and was lower overall. (11C-11D) Rainbow heatmaps of (11C) heat map micrograph shows mean mitochondrial membrane potential and (11D) major oscillating frequency of ΔΨm. Images show a correlation between areas with high mean mitochondrial membrane potential and oscillation frequency. (11E) Immunofluorescent micrograph of mitochondrial membrane potential (ΔΨm) measured using live imaging of JC-1 dye. (11F) Kinetic analysis of mitochondrial membrane potential (ΔΨm) following aggregation of JC-1 dye. Mitochondrial membrane potential showed distinct polarization peaks in contracting cells, while non-beating cells did not oscillate and show lower mitochondrial membrane potential overall. (11G-11H) Rainbow heatmaps of (11G) mean mitochondrial membrane potential and (11H) major oscillating frequency of ΔΨm measured using JC-1 (see Materials and methods). Similar to the behavior measured by TMRE, JC-1 heatmaps suggest a correlation between areas with high mean mitochondrial membrane potential and oscillation frequency. (11I) Live imaging of hiPSC-derived cardiomyocytes in 2D culture showed rapid oscillation in mitochondrial calcium [Ca²⁺]ₘ in beating cells, precisely correlated to the contraction frequency of the cells. (11J) Immunofluorescent micrographs of dynamic changes in mitochondrial calcium content following treatment with 10 μM mitochondrial calcium uniporter (MCU) inhibitor KB-R7943. Mitochondrial calcium was measured using live imaging of Rhod-2AM dye, mitochondrial content was measured by MitoTracker stain, and contraction was measured with visual contraction analysis (see Materials and methods). (11K) Representative single contraction measurements of mitochondrial calcium and contraction of cardiomyocytes following treatment with 10 μM KB-R7943. Acute MCU inhibition decreases the magnitude of cardiomyocyte contraction and its dependence on the efficient oscillation of mitochondrial calcium. (11L) Dynamic analysis of mitochondrial calcium amplitude and contractility of cardiomyocytes following treatment with 10 μM KB-R7943. The analysis suggests that the amplitude of mitochondrial calcium oscillation magnitude is correlated to the contractility, suggesting mitochondrial calcium oscillations is essential to maintain contraction magnitude. Bar=25 μm.

FIGS. 12A-12C include graphs and micrographs showing CRISPR/Cas9 knockout of MCU disrupts electro-mitochondrial coupling and induces arrhythmic behavior. (12A) Kinetic Rhod-2AM measurements of mitochondrial membrane potential (MMP) in beating 2D-cultured hiPSC-derived cardiomyocytes. Non-targeting sgRNA had no effect on [Ca²⁺]ₘ showing a dominant frequency of 0.8 Hz, while MCU knockout (MCU^{KO}) showed a 35-50% decrease in [Ca²⁺]ₘ and oscillation magnitude while increasing oscillation rate to 1.3-1.4 Hz. (12B-12C) (12B) RT-qPCR and (12C) Immunofluorescent confocal microscopy demonstrated a marked reduction in MCU expression on mRNA and protein levels. *p<0.05, **p<0.01, ***p<0.001. Error bars represent±S.E. Significance was determined using a two-tailed heteroscedastic student-test. Bar=100 μm.

FIGS. 13A-13E include micrographs, graphs, and schematics showing validation of electro-mitochondrial coupling in an ex-vivo porcine model. (13A) Schematic depicting the use of stimulated porcine left ventricle dissected tissue (see Materials and methods) embedded with oxygen sensors and placed on a multielectrode array as ex vivo validation model. (13B) Photo of dissected porcine tissue (green) on the electrode array. Bar=5 mm (13C) Scanning electron micrograph showing oxygen sensor (pseudo color) adhering to a porcine cardiac tissue section. Bar=25 μm (13D) Representative simultaneous kinetic measurements of contraction, field potential, and interstitial oxygen content in porcine cardiac tissue exposed to DMSO (control), 10 μM of blebbistatin, 10 μM mitoxantrone, or 10 μM mitoxantrone and 100 μM metformin (mitoxantrone+metformin). The myosin II inhibitor blebbistatin blocked cardiac contraction, without affecting field potential and oxygen oscillation frequency. Exposure to mitoxantrone induced arrhythmogenic behavior, increasing porcine tissue beating frequency from 1 to 2 Hz. Concurrent treatment with metformin partly reversed this effect in porcine tissue, decreasing beating frequency to 1.4 Hz. (13E) Continuous measurement of interstitial oxygen in porcine cardiac tissue exposed to mitoxantrone shows the kinetics of inhibition of mitochondrial activity. Treatment with metformin 50 minutes post-exposure shows a gradual restoration of function and reversal of the mitoxantrone's effect.

### DETAILED DESCRIPTION

The present invention, in some embodiments, provides methods of treating a disease or disorder characterized by electro-mitochondrial dyssynchronization. A multichambered cardiac organoid comprising endothelial cells, cardiomyocytes in a and at least two chambers beating in synchrony is also provided. Methods of producing and methods of using the multichambered cardiac organoid, as well sensing systems and their use are also provided.

### Cardiac Organoids

According to a first aspect, there is provided a cardiac organoid.

As used herein, an “organoid” refers to a simplified version of an organ produced in vitro. In some embodiments, the organoid is smaller than the in vivo organ. In some embodiments, the cardiac organoid is a cardiac organoid of the invention. In some embodiments, the organoid has a microanatomy or cellular organization similar to the organ. In some embodiments, the cellular organization is 2D organization. In some embodiments, the cellular organization is 3D cellular organization. In some embodiments, the organoid functions similarly to the organ. In some embodiments, the organoid has a gene expression similar to the organ. In some embodiments, gene expression is a gene expression profile. In some embodiments, the organoid beats similar to the organ. In some embodiments, the cells of the organoid are synchronized similar to the cells of the organ. In some embodiments, the organoid response to calcium similar to the organ. In some embodiments, the organoid has mitochondria that function similar to the organ's mitochondria. In some embodiments, the organoid signals similar to the organ. In some embodiments, the organoid responds to a drug or compound similar to the organ. In some embodiments, the organoid is useful for testing a drug to be used on the organ. In some embodiments, the organoid is useful for testing a side effect of a drug or compound of the organ. In some embodiments, the organoid is useful for modeling a disease of the organ. In some embodiments, the organ is a heart. In some embodiments, the organ is a portion of a heart. In some embodiments, the portion is a ventricle. In some embodiments, the portion is an atrium. In some embodiments, the organoid self-renews. In some embodiments, the organoid is not immortalized.

In some embodiments, the organoid is not genetically manipulated. In some embodiments, the organoid comprises diploid cells. In some embodiments, the organoid consists of diploid cells. In some embodiments, the organoid is devoid of aneuploid cells. In some embodiments, the organoid comprises cardiomyocytes. In some embodiments, the cardiomyocytes are derived from pluripotent stem cells. In some embodiments, the pluripotent stem cells are induced pluripotent stem cells (iPSCs). In some embodiments, the cardiomyocytes are obtained from PSCs. In some embodiments, the cardiomyocytes are human cardiomyocytes. In some embodiments, the iPSCs are human iPSCs. Methods of differentiation of pluripotent stem cells into cardiomyocytes are well known in the art and any such method may be employed. Further, an exemplary method is provided hereinbelow. In some embodiments, iPSCs are differentiated into cardiomyocytes.

As used herein, the term “cardiomyocyte(s)” refers to cardiac muscle cell(s) that make up a cardiac muscle (heart muscle). Each myocardial cell contains myofibrils, which are specialized organelles consisting of long chains of sarcomeres, the fundamental contractile units of muscle cells. Cardiomyocytes show striations similar to those on skeletal muscle cells. Unlike multinucleated skeletal cells, the majority of cardiomyocytes contain only one nucleus, although they may have as many as four. Cardiomyocytes have a high mitochondrial density, which allows them to produce adenosine triphosphate (ATP) quickly, making them highly resistant to fatigue.

By “induced pluripotent stem cell” or “iPSC” it is meant a PSC that is derived from a cell that is not a PSC (i.e., from a cell this is differentiated relative to a PSC), iPSCs can be derived from multiple different cell types, including terminally differentiated cells. iPSCs have an ES cell-like; morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs may be found in, for example, US. Patent Publication Nos, US20090047263, US20090068742, US20090191159, US20090227032, US20090246875, and US20090304646. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

In some embodiments, the organoid comprises endothelial cells. In some embodiments, the endothelial cells are microvascular endothelial cells. In some embodiments, the endothelial cells are cardiac endothelial cells. In some embodiments, the cardiac organoid comprises endothelial cells in a geometrically confined compartment. In some embodiments, the cells of the organoid are in a geometrically confined compartment. In some embodiments, the organoid comprises a mix of cardiomyocytes and endothelial cells. In some embodiments, the mix comprises a ratio of about 2:1 cardiomyocytes to endothelial cells.

In some embodiments, the organoid comprises at least 100, 1000, 5000, 10000, 15000, 20000, 25000, 30000, 35000, 40000, 45000, 50000, 55000, 60000, 65000, 68000, 70000, 75000, 80000, 85000, 90000, 95000 or 100000 cells. Each possibility represents a separate embodiment of the invention. In some embodiments, the organoid comprises at least 68000 cells. In some embodiments, the organoid comprises at least 75000 cells. In some embodiments, the organoid comprises at most 65000, 70000, 75000, 80000, 85000, 90000, 95000, 100000, 125000, 150000, 175000, 200000, 300000, 400000, 500000, 1000000, 10000000, 100000000, or 1000000000 cells. Each possibility represents a separate embodiment of the invention.

In some embodiments, the cardiac organoid is a multichambered cardiac organoid. In some embodiments, the organoid is a multichambered organoid. In some embodiments, multichambered comprises at least 2 chambers. In some embodiments, multichambered comprises at least 3 chambers. In some embodiments, multichambered comprises at least 4 chambers. In some embodiments, the chambers comprise chamber walls. In some embodiments, a chamber wall comprises endocardium. In some embodiments, a chamber wall is endocardium. In some embodiments, the chamber walls are capable of beating. In some embodiments, the organoid is a beating organoid. In some embodiments, beating is spontaneous beating. In some embodiments, the chamber walls are capable of contracting. In some embodiments, the beating or contracting is in response to a stimulus. In some embodiments, the stimulus is an endogenous stimulus. In some embodiments, the stimulus is an exogenous stimulus. In some embodiments, the stimulus is an endocrine or hormonal stimulus. In some embodiments, the stimulus is a chemical stimulus. In some embodiments, the stimulus is an electrical stimulus. In some embodiments, the organoid beats spontaneously and also beats, or alters beating, in response to a stimulus. In some embodiments, the beating is in synchrony.

In some embodiments, the cardiac organoid further comprises a vascular structure or a plurality thereof. In some embodiments, the cardiac organoid comprises endothelial cells. In some embodiments, addition of endothelial cells forms a vascular structure. In some embodiments, the structure forms naturally from the added endothelial cells without further stimulus. In some embodiments, the structure forms naturally from the added endothelial cells after addition of a pro-angiogenic stimulus. Pro-angiogenic proteins are well known in the art and any may be used. In some embodiments, the stimulus is a protein. In some embodiments, the protein is VEGF. In some embodiments, VEGF is VEGF-A. In some embodiments, a vascular structure includes or is selected from: artery, arteriole, capillary, vena, vein, venule, sinus, or any combination thereof. In some embodiments, the vascular structure comprises capillaries. In some embodiments, a wall of the chamber comprises capillaries. In some embodiments, the organoid comprises an anisotropic stress gradient. In some embodiments, the organoid comprises region of low-stress. In some embodiments, the vasculature forms in response to anisotropic stress.

In some embodiments, the cardiac organoid comprises circumferentially aligned cardiomyocytes. In some embodiments, the circumferentially aligned cardiomyocytes surround the chambers. In some embodiments, the chambers are hollow. In some embodiments, the chambers comprise fluid. In some embodiments, the fluid is culture media. In some embodiments, the chambers are surrounded by cardiomyocytes. In some embodiments, the chambers are layered with cardiomyocytes. In some embodiments, the walls of the chambers comprise circumferentially aligned cardiomyocytes. In some embodiments, the circumference is the circumference of the chamber. In some embodiments, the circumference is the circumference of the cardiomyocytes. In some embodiments, a chamber of the cardiac organoid comprises at least one capillary within a wall surrounding the chamber. In some embodiments, the chambers are surrounded by endocardium. In some embodiments, the endocardium is surrounded by epicardium.

In some embodiments, the cardiac organoid comprises elongated cardiomyocytes. In some embodiments, the cardiomyocytes are elongated cardiomyocytes. In some embodiments, the cardiomyocytes are organized in a sarcomeric pattern. In some embodiments, the cardiomyocytes are organized into sarcomeres. As used herein, the term “sacromeric pattern” refers to being organized similarly, or essentially the same as a sarcomere, e.g., the smallest functional unit of striated muscle or muscle tissue. In some embodiments, the cardiomyocytes are α-actinin positive. In some embodiments, the cardiomyocytes are cardiac troponin positive. The structure of muscle sarcomere as well as methods of determining same, such as by histology and/or microscopy, are common and would be apparent to a person of ordinary skill in the art.

In some embodiments, the cardiac organoid comprises at least one fibroblast-like cell. In some embodiments, the fibroblast-like cell is a fibroblast. In some embodiments, the fibroblast-like cell is a cardiac fibroblast-like cell. In some embodiments, the cardiac organoid comprises cardiac fibroblast-like cells. In some embodiments, the cardiomyocyte layer comprises fibroblast-like cells. In some embodiments, a chamber wall comprises a fibroblast-like cell. Fibroblast-like cells exhibit structural features and antigenic profiles related to their specific locations and functions. Non-limiting examples of fibroblast-like cells include, but are not limited to, myofibroblasts, perineural sheath cells, Ito cells, endocrine fibroblast-like cells, pericryptal and villous fibroblasts in the intestine, fibroblast-like cells in the myenteric plexus, lymphoid organ dendritic cells and fibroblasts at different sites including tendon, dermis and cornea.

In some embodiments, fibroblast-like cells are Periostin (POSTN) positive cells. In some embodiments, fibroblast-like cells are characterized by POSTN expression. In some embodiments, fibroblast-like cells comprise POSTN mRNA, a protein product thereof, or both. Methods of identifying fibroblast-like cells and markers for doing such are well known to the skilled artisan. In some embodiments, positive is protein positive. In some embodiments, positive is mRNA positive. In some embodiments, positive is expression positive.

In some embodiments, the cardiac organoid comprises a pacemaker-like cell. In some embodiments, the pacemaker-like cell is in a cluster. In some embodiments, the cardiac organoid comprises pacemaker-like cell clusters. In some embodiments, the pacemaker-like cell is Potassium/sodium hyperpolarization-activated cyclic nucleotide-gated channel 4 (HCN4) positive. In some embodiments, the pacemaker-like cell cluster is HCN4 positive. In some embodiments, the pacemaker-like cell is Short-stature homeobox 2 (SHOX2) positive. In some embodiments, the pacemaker-like cell cluster is SHOX2 positive. In some embodiments, the pacemaker-like cell is HCN4 and SHOX2 double positive. In some embodiments, the pacemaker-like cell cluster is HCN4 and SHOX2 double positive. In some embodiments, the pacemaker-like cell cluster comprise: HCN4 mRNA, a protein product thereof, or both, SHOX2 mRNA, a protein product thereof, or both, or any combination thereof. In some embodiments, a pacemaker-like cell is a pacemaker cell.

In some embodiments, the fibroblast-like cell is in contact with the pacemaker-like cell. In some embodiments, the cardiomyocytes are in contact with the pacemaker-like cell. In some embodiments, the contact with the pacemaker-like cell is in the cardiomyocyte layer. In some embodiments, the chamber wall comprises the pacemaker-like cell.

Methods for determining “positiveness” to a factor, or expression, as described herein, are common and would be apparent to one of ordinary skill in the art. Non-limiting examples for methods for determining expression include, but are not limited to, PCR, RT-PCR, quantitative RT-PCR, northern blot, RNA in situ hybridization, dot blot, western blot, or others.

In some embodiments, the cardiac organoid comprises an epicardium. In some embodiments, the epicardium is an outer epicardium. In some embodiments, the outer epicardium is an outer shell. In some embodiments, the chambers and chamber walls are surrounded by an epicardium. In some embodiments, the cardiomyocyte layer is surrounded by an epicardium. In some embodiments, the epicardium comprises cells positive for Wilms' tumor-1 (WT1). In some embodiments, the epicardium comprises cells positive for T-box transcription factor 18 (TBX18). In some embodiments, the epicardium comprises cells positive for WT1 and TXB18. In some embodiments, the epicardium cells comprise: WT1 mRNA, a protein product thereof, or both, TBX18 mRNA, a protein product thereof, or both, or any combination thereof.

In some embodiments, the chamber walls comprise endocardial-like cells. In some embodiments, the chamber walls comprise endocardium. In some embodiments, the endocardial-like cells are Platelet endothelial cell adhesion molecule (PECAM-1) positive. In some embodiments, the endocardium comprises cells positive for PECAM-1. In some embodiments, the endocardium is inner endocardium. In some embodiments, the organoid comprises an inner endocardium. In some embodiments, the organoid comprises and inner endocardium and an outer epicardium. In some embodiments, the chamber walls comprise PECAM-1 mRNA, a protein product thereof, or both. In some embodiments, the organoid comprises endocardial-like cell layer. In some embodiments, the chamber is layered with an endocardial-like cell layer. In some embodiments, a portion of the chamber is layered with an endocardial-like cell layer.

In some embodiments, the organoid comprises a ring of high stress cell. In some embodiments, high stress cells are high stress regions. In some embodiments, the high stress is marked by Lamanin A/C (LMNA). In some embodiments, the organoid comprises a gradient of Yes-associated protein 1 (YAP1). In some embodiments, the gradient surrounds a central cavity or chamber.

In some embodiments, the cardiac organoid disclosed herein is capable of beating. In some embodiments, the cardiac organoid disclosed herein is characterized by being capable of synchronized beating. In some embodiments, the cardiac organoid beats in synchrony. In some embodiments, the cardiac organoid comprises chambers beating in synchrony. In some embodiments, at least two chambers of the organoid beat in synchrony. In some embodiments, all chambers of the organoid beat in synchrony. In some embodiments, the organoid produces multi-phasic beating. In some embodiments, the organoid comprises multi-phasic beating. In some embodiments, multi-phasic is bi-phasic. In some embodiments, the organoid comprises at least a first two chambers beating in synchrony and at least a second two chambers beating in synchrony. In some embodiments, the first two chambers and the second two chambers are not synchronized. In some embodiments, the first two chambers and the second two chambers beat at different times. In some embodiments, beating at different times comprises a synchronization of the two groups of chambers such that when one beats the other does not beat and vice-versa. In some embodiments, the beating is spontaneous beating. In some embodiments, synchronized beating refers to at least 50%, at least 60%, at least 70%, at least 80%, at least 90%, at least 95%, at least 99%, or 100% of the cells or the contracting cells of the cardiac organoid disclosed herein, contracting simultaneously. Each possibility represents a separate embodiment of the invention. In some embodiments, the synchronous contraction is in response to a stimulus. In some embodiments, the cells all respond to the same stimulus.

In some embodiments, the synchronized beating persists for at least 1, 2, 3, 4, 5 or more weeks in culture, or any value and range therebetween. Each possibility represents a separate embodiment of the invention. In some embodiments, the organoid is capable of synchronized beating for at least 1, 2, 3, 4, 5 or more weeks in culture, or any value and range therebetween. Each possibility represents a separate embodiment of the invention. In some embodiments, the synchronized beating persists for 1-10 weeks, 2-12 weeks, 3-15 weeks, 4-10 weeks, 5-25 weeks, in culture. Each possibility represents a separate embodiment of the invention. In some embodiments, the synchronized beating is for at least 1 week in culture.

In some embodiments, beating is at a rate of least 20 beats per minute (bpm), at least 30 bpm, at least 35 bpm, at least 40 bpm, at least 45 bpm, at least 50 bpm, at least 55 bpm, at least 60 bpm, at least 65 bpm, at least 70 bpm, at least 75 bpm, at least 80 bpm, at least 85 bpm, at least 88 bpm, at least 90 bpm or any value and range therebetween. Each possibility represents a separate embodiment of the invention. In some embodiments, the beating is at a rate of at least 50 bpm. In some embodiments, the beating is at a rate of at least 52 bpm. In some embodiments, the beating is at a rate of at least 60 bpm. In some embodiments, the beating is at a rate of at least 65 bpm. In some embodiments, the beating is about 66 bpm. In some embodiments, the beating is at a rate of at least 88 bpm. In some embodiments, the beating is about 88 bpm. In some embodiments, the organoid beats at a rate similar to the organ. In some embodiments, the organoid beating rate is an unstimulated beating rate. It will be understood that the addition of a stimulant or other agent may increase or decrease the rate of beating, but the standard/untreated beating rate will be as indicated.

In some embodiments, the beating is between 20 and 90 bpm, 30 and 90 bpm, 40 and 90 bpm, 50 and 90 bpm, 60 and 90 bpm, 55 and 90 bpm, 45 and 90 bpm, 20 and 88 bpm, 30 and 88 bpm, 40 and 88 bpm, 50 and 88 bpm, 60 and 88 bpm, 55 and 88 bpm, 45 and 88 bpm, 20 and 80 bpm, 30 and 80 bpm, 40 and 80 bpm, 50 and 80 bpm, 60 and 80 bpm, 55 and 80 bpm, 45 and 80 bpm, 35 and 90 bpm, 55 and 80 bpm, or 55 and 75 bpm. Each possibility represents a separate embodiment of the invention. In some embodiments, the beating is between 40 and 80 bpm. In some embodiments, the beating is between 50 and 80 bpm. In some embodiments, the beating is between 40 and 90 bpm. In some embodiments, the beating is between 50 and 90 bpm. In some embodiments, the organoid responds to treatment with a stimulant by increasing beat rate. In some embodiments, the organoid responds to treatment with a stimulant by increasing contraction amplitude. Stimulants that increase heart rate are well known in the art and any such stimulant may be employed. In some embodiments, the stimulant is epinephrine. In some embodiments, the organoid responds to treatment with a potassium channel blocker by decreasing beat rate. In some embodiments, the organoid responds to treatment with a potassium channel blocker by decreasing contraction amplitude. In some embodiments, the organoid responds to treatment with an antiarrhythmic drug by decreasing beat rate. In some embodiments, the organoid responds to treatment with an antiarrhythmic drug by decreasing contraction amplitude. Drugs that decrease heart rate are well known in the art and any such drug may be used. In some embodiments, the drug that decreases heart rate and/or contraction amplitude is amiodarone.

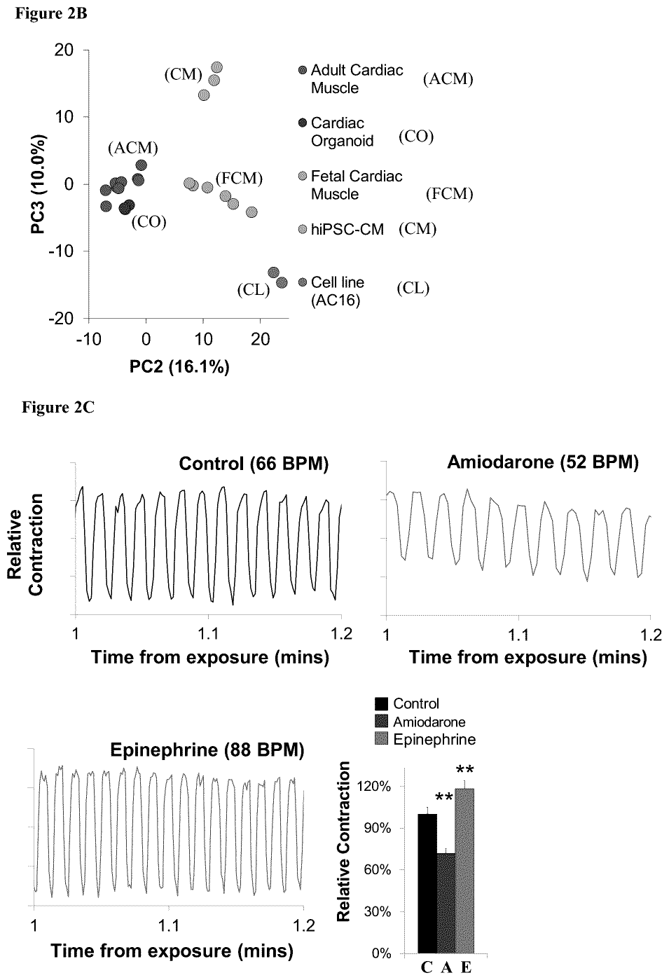

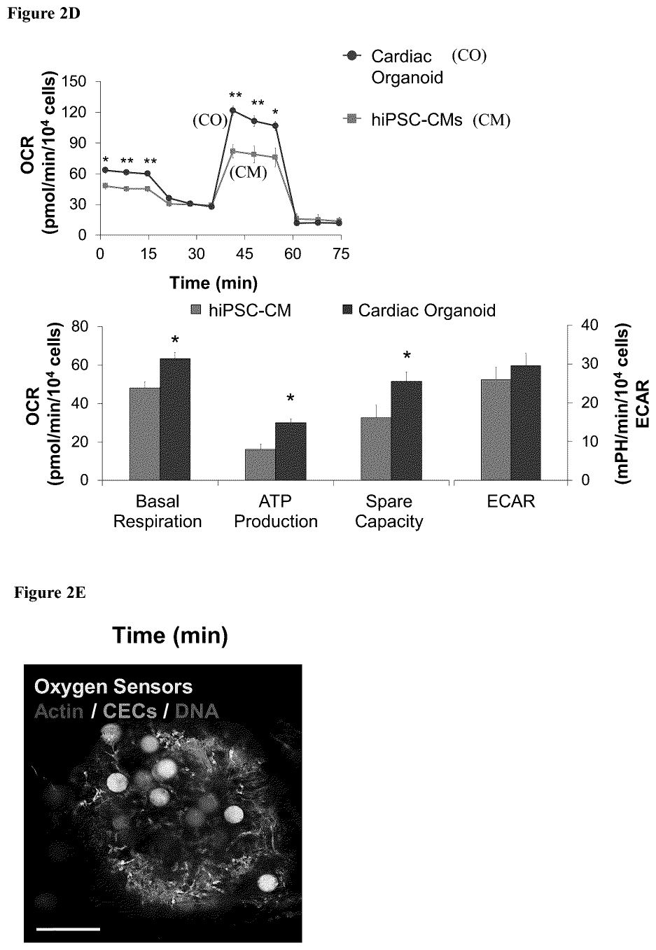

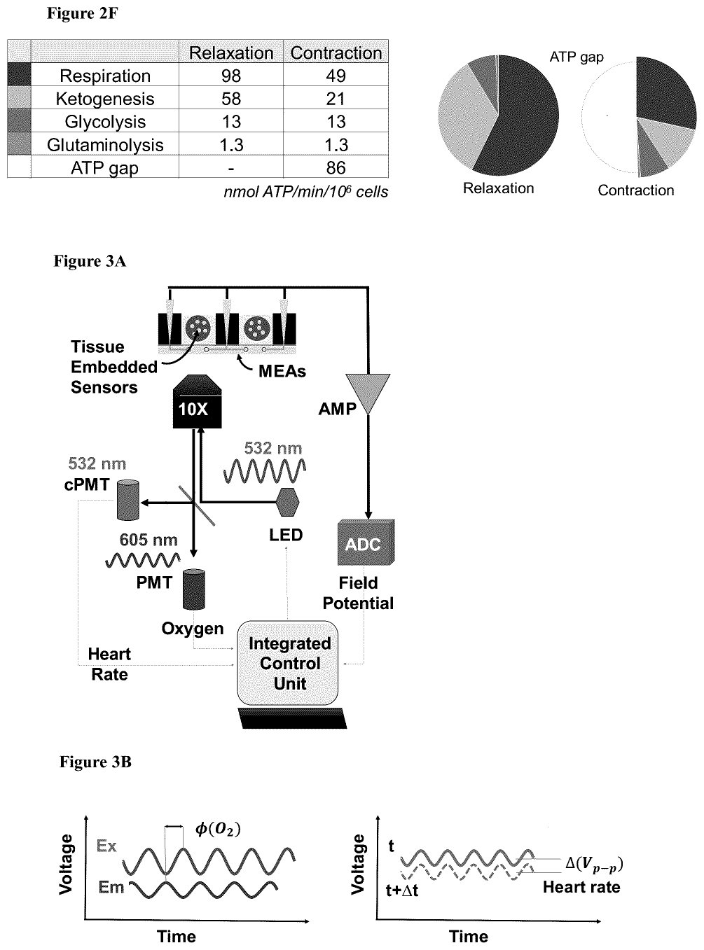

In some embodiments, the cardiac organoid disclosed herein comprises cells comprising increased expression of at least one factor of the factors provided in FIG. 2A. In some embodiments, the cardiac organoid disclosed herein comprises cells comprising increased expression of at least one factor selected from: Troponin T2 (TNNT2), Troponin I (TNNI3), Connexin 43 (Cx43, also known as GJA1), Myosin heavy chain 7 (MYH7), A-kinase anchoring protein 6 (AKAP6), Gap junction protein alpha 5 (GJA5), Junctophilin 2 (JPH2), Solute carrier family 8 member 1 (SLC8A1), ATPase Ca++ transporting cardiac muscle, slow twitch 2 (ATP2A2 also known as SERCA2), Calcium channel voltage-dependent L type alpha 1C subunit (CACNA1C), Ryanodine receptor 2 (RYR2), Calsequestrin 2 (CASQ2), Phospholamban (PLN, also known as PLB), Calcium/calmodulin-dependent protein kinase type II beta chain (CAMK2B), Triadin (TRDN), Caveolin (CAV3), Myc box-dependent-interacting protein 1 (BIN1), Amphiphysin (AMP2 also known as AMPH), Sodium channel protein type 5 subunit alpha (SCN5A), Potassium voltage-gated channel subfamily J member 2 (KIR2.1, also known as KCNJ2), Inositol 1,4,5-triphosphate receptor, type 3 (ITPR3), Potassium/sodium hyperpolarization-activated cyclic nucleotide-gated ion channel 2 (HCN2), Sodium channel subunit beta-1 (SCN1B), Potassium/sodium hyperpolarization-activated cyclic nucleotide-gated ion channel 1 (HCN1), Potassium inwardly-rectifying channel, subfamily J, member 8 (KCNJ8), Potassium voltage-gated channel subfamily H member 2 (KCNH2, also known as hERG), 5′-AMP-activated protein kinase catalytic subunit alpha-1 (PRKAA1), Carnitine palmitoyltransferase I (CPT1A), Mitochondrial transcription factor A (TFAM), Peroxisome proliferator-activated receptor gamma coactivator 1-alpha (PPARGCIA), Inorganic Pyrophosphatase 1 (PPA1), Serine/threonine-protein phosphatase 2A regulatory subunit B (PPP2R4), Glucose transporter type 4 (SLC2A4, also known as GLUT4), Mitogen-activated protein kinase 1 (MAPK1, also known as ERK2), Catalytic subunit a of protein kinase A (PRKACA), Alpha-1A adrenergic receptor (α1A, also known as ADRA1A), alpha-1B adrenergic receptor (alB, also known as ADRAIB), Sodium channel β-subunit 4 (SCN4B), and Potassium voltage-gated channel subfamily E member 1 (KCNE1). In some embodiments, the cardiac organoid disclosed herein is characterized by or comprises increased expression of at least one factor selected from: TNNT2, TNNI3, Cx43, MYH7, AKAP6, GJA5, JPH2, SLC8A1, ATP2A2, CACNA1C, RYR2, CASQ2, PLN, CAMK2B, TRDN, CAV3, BIN1, AMP2, SCN5A, KIR2.1, ITPR3, HCN2, SCN1B, HCN1, KCNJ8, KCNH2, PRKAA1, CPT1A, TFAM, PPARGC1A, PPA1, PPP2R4, SLC2A4, MAPK1, PRKACA, α1A, α1B, SCN4B, and KCNE1. In some embodiments, increased is as compared to isolated cardiomyocytes. In some embodiments, increased is as compared to cardiomyocyte culture. In some embodiments, the cardiomyocytes are the same cardiomyocytes as used to produce the organoid. In some embodiments, the cardiomyocytes are derived from iPSCs. In some embodiments, increased is as compared to fetal cardiac tissue. In some embodiments, the organoid comprises gene expression more similar to adult cardiac muscle than fetal cardiac muscle. In some embodiments, the cardiac organoid disclosed herein comprises cells comprising increased expression of at least 1, 2, 3, 4, 5, 6, 7, 8, 9 or all of TNNT2, TNNI3, Cx43, MYH7, AKAP6, GJA5, JPH2, SLC8A1, ATP2A2, CACNA1C, RYR2, CASQ2, PLN, CAMK2B, TRDN, CAV3, BIN1, AMP2, SCN5A, KIR2.1, ITPR3, HCN2, SCN1B, HCN1, KCNJ8, KCNH2, PRKAA1, CPT1A, TFAM, PPARGC1A, PPA1, PPP2R4, SLC2A4, MAPK1, PRKACA, α1A, α1B, SCN4B, and KCNE1. Each possibility represents a separate embodiment of the invention. In some embodiments, the cardiac organoid disclosed herein is characterized by or comprises increased expression of at least 1, 2, 3, 4, 5, 6, 7, 8, 9 or all of TNNT2, TNNI3, Cx43, MYH7, AKAP6, GJA5, JPH2, SLC8A1, ATP2A2, CACNA1C, RYR2, CASQ2, PLN, CAMK2B, TRDN, CAV3, BIN1, AMP2, SCN5A, KIR2.1, ITPR3, HCN2, SCN1B, HCN1, KCNJ8, KCNH2, PRKAA1, CPT1A, TFAM, PPARGC1A, PPA1, PPP2R4, SLC2A4, MAPK1, PRKACA, α1A, α1B, SCN4B, and KCNE1. Each possibility represents a separate embodiment of the invention.

In some embodiments, increased expression is increased mRNA expression. In some embodiments, increased expression is increased protein expression. In some embodiments, increased expression comprises increased levels or amounts of mRNA molecules, protein molecules, or both, or at least one factor, as disclosed herein. In some embodiments, increased is at least: 10, 15, 20, 23, 30, 35, 40, 45, 50, 55, 60, 65, 70, 75, 80, 85, 90, 95, 100, 110, 120, 125, 130, 140, 150, 160, 170, 180, 190, 200, 250, 300, 350, 400, 40, or 500% increased, or any value and range therebetween. Each possibility represents a separate embodiment of the invention. In some embodiments, increased is 10-1,000%, 150-900%, 200-990%, 300-1,200%, 500-975%, or 600-1,100%. Each possibility represents a separate embodiment of the invention.

In some embodiments, the cardiac organoid produces a physiological response to a condition. In some embodiments, the condition is an induced condition. In some embodiments, the condition is a physiological condition or parallel to a physiological condition. In some embodiments, the condition is the application, administration or contact with a therapeutic agent. In some embodiments, the therapeutic agent is a drug. In some embodiments, the condition is the application, administration or contact with a chemical. In some embodiments, the chemical is a harmful chemical. In some embodiments, the chemical is a toxin. In some embodiments, the chemical is a solvent. In some embodiments, the solvent is an organic solvent. Examples of chemicals that can cause heart disease/disorder or side effects can be found for example is Kurppa et al., 1984 “Chemical exposures at work and cardiovascular morbidity”, Scand. J. Work Environ. Healthy 10: 381-388, Assadi, 2017, “Electrocardiographic changes and exposure to solvents”, J Arrhythm., December 14; 34(1):65-70 and Tsutsumi, 2015, “Prevention and management of work-related cardiovascular disorders”, Int J Occup Med Environ Health; 28(1):4-7, herein incorporated by reference in their entirety.

In some embodiments, the condition is hypoxia. In some embodiments, the condition is a hypoxic condition. Hypoxia is a situation of low oxygen for a cell. In some embodiments, hypoxia comprises oxygen levels below 6, 5, 4, 3, 2.5, 2, 1.5, 1, 0.75, 05. 0.25 or 0.1% oxygen. Each possibility represents a separate embodiment of the invention. In some embodiments, hypoxia comprises 2% oxygen or less. In some embodiments, the condition is a circulation condition. In some embodiments, the condition is the application of circulation. In some embodiments, the circulation is irregular or abnormal circulation. In some embodiments, irregular circulation is low circulation. In some embodiments, irregular circulation is high circulation. In some embodiments, high, low, regular and abnormal are as compared to circulation in a healthy heart. In some embodiments, low circulation comprises low fluid flow over the organoid and/or high circulation comprises high fluid flow over the organoid. In some embodiments, low circulation is an ischemic condition. In some embodiments, the condition is an ischemic condition. In some embodiments, low circulation comprises low nutrients. In some embodiments, the nutrient is glucose. In some embodiments, the nutrient is an amino acid. In some embodiments, the condition is exposure to a metabolite. In some embodiments, the condition is a change in metabolite exposure. In some embodiments, the condition is withdrawal of a metabolite. In some embodiments, a metabolite is glucose. In some embodiments, the condition is exposure to a hormone. In some embodiments, the condition is a change in hormone exposure. In some embodiments, the condition is withdrawal of a hormone. In some embodiments, the condition is a genetic mutation of the cells of the organoid. In some embodiments, the condition mimics diabetes, ischemia, genetic disease or any other condition, disease or damage. In some embodiments, the cardiac organoid produces a physiological response to a condition, that is essentially the same as cardiac tissue, or a heart. In some embodiments, the cardiac organoid disclosed herein is a heart simulating organoid.

In some embodiments, the cardiac organoid disclosed herein comprises cells comprising increased oxidative phosphorylation. In some embodiments, the cardiac organoid disclosed herein is characterized by or comprises increased oxidative phosphorylation. In some embodiments, the cardiac organoid disclosed herein comprises cells comprising increased basal respiration. In some embodiments, the cardiac organoid disclosed herein is characterized by or comprises increased basal respiration. In some embodiments, the cardiac organoid disclosed herein comprises cells comprising increased mitochondrial maximal capacity. In some embodiments, the cardiac organoid disclosed herein is characterized by or comprises increased mitochondrial maximal capacity. In some embodiments, basal respiration is increased by at least 30%. In some embodiments, basal respiration is increased by at least 35%. In some embodiments, basal respiration is increased by at about 35%. In some embodiments, oxidative phosphorylation is increased by at least 80%. In some embodiments, oxidative phosphorylation is increased by at least 85%. In some embodiments, oxidative phosphorylation is increased by about 85%. In some embodiments, mitochondrial maximal capacity is increased by at least 90%. In some embodiments, mitochondrial maximal capacity is increased by at least 100%. In some embodiments, mitochondrial maximal capacity is increased by at least 200%. In some embodiments, mitochondrial maximal capacity is increased by about 2-fold. In some embodiments, mitochondrial maximal capacity is increased by about 100%.

In some embodiments, the organoid further comprises oxygen sensing particles. In some embodiments, the particles are beads. In some embodiments, the particles are synthetic. In some embodiments, the particles are non-organic. In some embodiments, the particles are embedded in the organoid. In some embodiments, the organoid further comprises a sensor. In some embodiments, the sensor is an electrochemical sensor. In some embodiments, the sensor is a glucose sensor. In some embodiments, the sensor is a lactate sensor. In some embodiments, the sensor is a glutamine sensor. In some embodiments, the sensor is configured to measure or sense at least one or glucose, lactate or glutamine. In some embodiments, the organoid is characterized by fatty acid oxidation as the primary metabolic pathway. In some embodiments, the organoid is characterized by changes in interstitial oxygen at sub-second resolution.

### Methods of Production

According to another aspect, there is provided a method of producing a cardiac organoid.

According to another aspect, there is provided a cardiac organoid produced by a method of the invention.

In some embodiments, the method comprises coculturing cardiomyocytes and endothelial cells. In some embodiments, the coculturing is coculturing a mass of cardiomyocytes and endothelial cells. In some embodiments, the endothelial cells are microvascular cells. In some embodiments, the endothelial cells are cardiac cells. Microvascular cardiac endothelial cells are available commercially, or can be produced by any method known in the art. In some embodiments, the cardiomyocytes are derived from iPSCs. In some embodiments, the organoid is a human organoid and the cardiomyocytes are human cells. In some embodiments, the organoid is a human organoid and the endothelial cells are human cells. In some embodiments, the method further comprises differentiating iPSCs into cardiomyocytes. In some embodiments, the differentiation is as described hereinbelow. It will be understood that any differentiation method known in the art may be used.

In some embodiments, the cardiomyocytes and endothelial cells are cocultured in a ratio of about 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 4:1, 5:1, 6:1, 7:1, 8:1, 9:1 or 10:1. Each possibility represents a separate embodiment of the invention. In some embodiments, the cardiomyocytes and endothelial cells are cocultured in a ratio of about 2:1. In some embodiments, the cardiomyocytes and endothelial cells are cocultured in a ratio of at least 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, or 2.5:1. Each possibility represents a separate embodiment of the invention. In some embodiments, the cardiomyocytes and endothelial cells are cocultured in a ratio of at least 2:1. In some embodiments, the cardiomyocytes and endothelial cells are cocultured in a ratio of at between 1:1 to 3:1, 1.1:1 to 3:1, 1.2:1 to 3:1, 1.3:1 to 3:1, 1.4:1 to 3:1, 1.5:1 to 3:1, 1.6:1 to 3:1, 1.7:1 to 3:1, 1.8:1 to 3:1, 1.9:1 to 3:1, 2:1 to 3:1, 1:1 to 2.9:1, 1.1:1 to 2.9:1, 1.2:1 to 2.9:1, 1.2.9:1 to 2.9:1, 1.4:1 to 2.9:1, 1.5:1 to 2.9:1, 1.6:1 to 2.9:1, 1.7:1 to 2.9:1, 1.8:1 to 2.9:1, 1.9:1 to 2.9:1, 2:1 to 2.9:1, 1:1 to 2.8:1, 1.1:1 to 2.8:1, 1.2:1 to 2.8:1, 1.3:1 to 2.8:1, 1.4:1 to 2.8:1, 1.5:1 to 2.8:1, 1.6:1 to 2.8:1, 1.7:1 to 2.8:1, 1.8:1 to 2.8:1, 1.9:1 to 2.8:1, 2:1 to 2.8:1, 1:1 to 2.7:1, 1.1:1 to 2.7:1, 1.2:1 to 2.7:1, 1.3:1 to 2.7:1, 1.4:1 to 2.7:1, 1.5:1 to 2.7:1, 1.6:1 to 2.7:1, 1.7:1 to 2.7:1, 1.8:1 to 2.7:1, 1.9:1 to 2.7:1, 2:1 to 2.7:1, 1:1 to 2.6:1, 1.1:1 to 2.6:1, 1.2:1 to 2.6:1, 1.3:1 to 2.6:1, 1.4:1 to 2.6:1, 1.5:1 to 2.6:1, 1.6:1 to 2.6:1, 1.7:1 to 2.6:1, 1.8:1 to 2.6:1, 1.9:1 to 2.6:1, 2:1 to 2.6:1, 1:1 to 2.5:1, 1.1:1 to 2.5:1, 1.2:1 to 2.5:1, 1.3:1 to 2.5:1, 1.4:1 to 2.5:1, 1.5:1 to 2.5:1, 1.6:1 to 2.5:1, 1.7:1 to 2.5:1, 1.8:1 to 2.5:1, 1.9:1 to 2.5:1, 2:1 to 2.5:1, 1:1 to 2.4:1, 1.1:1 to 2.4:1, 1.2:1 to 2.4:1, 1.3:1 to 2.4:1, 1.4:1 to 2.4:1, 1.5:1 to 2.4:1, 1.6:1 to 2.4:1, 1.7:1 to 2.4:1, 1.8:1 to 2.4:1, 1.9:1 to 2.4:1, 2:1 to 2.4:1, 1:1 to 2.3:1, 1.1:1 to 2.3:1, 1.2:1 to 2.3:1, 1.3:1 to 2.3:1, 1.4:1 to 2.3:1, 1.5:1 to 2.3:1, 1.6:1 to 2.3:1, 1.7:1 to 2.3:1, 1.8:1 to 2.3:1, 1.9:1 to 2.3:1, 2:1 to 2.3:1, 1:1 to 2.2:1, 1.1:1 to 2.2:1, 1.2:1 to 2.2:1, 1.3:1 to 2.2:1, 1.4:1 to 2.2:1, 1.5:1 to 2.2:1, 1.6:1 to 2.2:1, 1.7:1 to 2.2:1, 1.8:1 to 2.2:1, 1.9:1 to 2.2:1, 2:1 to 2.2:1, 1:1 to 2.1:1, 1.1:1 to 2.1:1, 1.2:1 to 2.1:1, 1.3:1 to 2.1:1, 1.4:1 to 2.1:1, 1.5:1 to 2.1:1, 1.6:1 to 2.1:1, 1.7:1 to 2.1:1, 1.8:1 to 2.1:1, 1.9:1 to 2.1:1, 2:1 to 2.1:1, 1:1 to 2:1, 1.1:1 to 2:1, 1.2:1 to 2:1, 1.3:1 to 2:1, 1.4:1 to 2:1, 1.5:1 to 2:1, 1.6:1 to 2:1, 1.7:1 to 2:1, 1.8:1 to 2:1, or 1.9:1 to 2:1. Each possibility represents a separate embodiment of the invention. In some embodiments, the cardiomyocytes and endothelial cells are cocultured in a ratio of at between 1.5:1 to 2.5:1. In some embodiments, a coculture is a mixture.

In some embodiments, the coculture is in a 3D scaffold. In some embodiments, the coculture is in media sufficient for the formation of 3D cell structure. In some embodiments, the coculture is in a cell culture matrix. In some embodiments, the coculture is in a basement membrane matrix. In some embodiments, the matrix is solubilized. In some embodiments, the matrix is Matrigel. In some embodiments, the Matrigel is growth factor reduced Matrigel. In some embodiments, the Matrigel is devoid of growth factors. In some embodiments, growth factors are supplemented growth factors. In some embodiments, the cells are suspended in the matrix/scaffold.

In some embodiments, the cocultured cells are cocultured at a density of about 6.8×10⁴ cells per μl. In some embodiments, the cocultured cells are cocultured at a density of about 1, 2, 3, 4, 4.5, 5, 5.5, 6, 6.1, 6.2, 6.3, 6.4, 6.5, 6.6, 6.7, 6.8, 6.9, 7, 7.1, 7.2, 7.3, 7.4, 7.5, 7.6, 8, 9, or 10×10⁴ cells per 1l. Each possibility represents a separate embodiment of the invention. In some embodiments, the cocultured cells are seeded at a density of about 6.8×10⁴ cells per μl. In some embodiments, the cocultured cells are cocultured at a density of at least 1, 2, 3, 4, 4.5, 5, 5.5, 6, 6.1, 6.2, 6.3, 6.4, 6.5, 6.6, 6.7, 6.8×10⁴ cells per μl. Each possibility represents a separate embodiment of the invention. In some embodiments, the cocultured cells are cocultured at a density of at most 6.8, 6.9, 7, 7.1, 7.2, 7.3, 7.4, 7.5, 7.6, 8, 9, 10, 20, 30, 40, 50, 60, 70, 80, 90 or 100×10⁴ cells per μl. Each possibility represents a separate embodiment of the invention.

In some embodiments, the coculture is in a microwell. In some embodiments, the coculture is transferred to a microwell. In some embodiments, the coculture is allowed to expand in a microwell. In some embodiments, the coculture is allowed to form an organoid in the microwell. In some embodiments, the coculture is allowed to coalesce in the microwell. In some embodiments, the mixture of cardiomyocytes and endothelial cells are cocultured in a microwell. In some embodiments, the microwell is of a size to allow growth of the cells at the seeding density. In some embodiments, the microwell is of a size to allow growth of a given number of cells. In some embodiments, the microwell comprises a diameter of about 1 mm. In some embodiments, the microwell comprises a diameter of about 1.2 mm. In some embodiments, the microwell comprises a diameter of about 1.5 mm. In some embodiments, the microwell comprises a diameter of between 1-1.5 mm. It will be understood by a skilled artisan that while a microwell of 1-1.5 mm is a sufficient size for 7.48×10\hat{ }4 cells contained in about 1.1 ul of Matrigel the size of the organoid can be scaled up or down by increasing the volume of the microwell and the volume of scaffold/cells proportionately.

In some embodiments, the coculture is in a geometrically confined space. In some embodiments, the geometrically confined space is a microwell. In some embodiments, the coculturing in a geometrically confined space is such that anisotropic stress gradients are generated in the cells. In some embodiments, the coculturing produces anisotropic stress gradients in the cells. In some embodiments, in the cells is in the cell mass. In some embodiments, the method comprises producing anisotropic stress gradients in the cells. In some embodiments, the anisotropic stress gradients are generated by coculturing in a geometrically confined space. In some embodiments, the space confines growth of the cells. In some embodiments, confining the cells comprises the cells growing to be in contact with the walls of the space. It will be understood by a skilled artisan that in order to for the forming organoid to be confined the size of the culture well/dish must be calibrated to the number of cells added. If too few cells are added, the culture is not confined and so will form a single mass of cells and not a multichambered structure due to the homogenous stress. Too many cells and the organoid cannot properly structurally organize as described herein. The same is true for cells in a scaffold (e.g., Matrigel). The size of the well must be calibrated to the volume of scaffold added.

In some embodiments, the cells in matrix/scaffold are cultured in media. In some embodiments, the media is culture media. In some embodiments, the media is tissue culture media. In some embodiments, the media is RPMI or an equivalent media. In some embodiments, the RPMI is RPMI-1640. Tissue culture media are well known and which media are equivalent would also be understood by a skilled artisan. In some embodiments, the media is media for non-adherent cells. In some embodiments, the media is non-adherent cell media. In some embodiments, the media is supplemented with B27. In some embodiments, the media is no supplemented with insulin. In some embodiments, the media is supplemented with a growth factor. In some embodiments, the growth factor is vascular endothelial growth factor (VEGF). In some embodiments, the VEGF is VEGF-A. In some embodiments, the growth factor is at a concentration of about 2, 2.5, 3, 3.5, 4, 4.5, 5, 5.5, 6, 6.5, 7, 7.5, or 8 ng/ml. Each possibility represents a separate embodiment of the invention. In some embodiments, the growth factor is at a concentration of at least 0.5, 1, 1.5, 2, 2.5, 3, 3.5, 4, 4.5, or 5, ng/ml. Each possibility represents a separate embodiment of the invention. In some embodiments, the growth factor is at a concentration of at most 5, 5.5, 6, 6.5, 7, 7.5, 8, 8.5, 9, 9.5, or 10 ng/ml. Each possibility represents a separate embodiment of the invention. In some embodiments, the growth factor is at a concentration of about 5 ng/ml. In some embodiments, the growth factor is at a concentration of between 2.5-7.5 ng/ml. In some embodiments, the growth factor is at a concentration of between 3-7 ng/ml. In some embodiments, the growth factor is at a concentration of between 4-6 ng/ml. In some embodiments, the growth factor is at a concentration of between 4.25-5.75 ng/ml. In some embodiments, the growth factor is at a concentration of between 4.5-5.5 ng/ml.

In some embodiments, the cells are cultured for a time sufficient for them to start beating. In some embodiments, beating is synchronous beating. In some embodiments, beating is spontaneous beating. In some embodiments, beating is beating such as produced by the organoid of the invention. In some embodiments, the cells are cultured for a time sufficient to produce an organoid of the invention. In some embodiments, the cells are cultured for a time sufficient to produce an organoid that displays or comprises at least one characteristic of an organoid of the invention. Characteristics of organoids of the invention are described herein above. In some embodiments, the cardiac organoid is a cardiac organoid of the invention. In some embodiments the method is a method of producing an organoid of the invention.

### Methods of Use

According to another aspect, there is provided a method for testing a therapeutic agent or compound, the method comprising contacting a cardiac organoid of the invention with the therapeutic agent or compound.

According to another aspect, there is provided a method of evaluating cardiac cell function, the method comprising exposing a cardiac organoid of the invention to a condition thereby evaluating cardiac cell function.

In some embodiments, the method is a diagnostic method. In some embodiments, the method is an in vitro method. In some embodiments, the method is an ex vivo method. In some embodiments, the method is a non-patient specific method. In some embodiments, the method is a culture method. In some embodiments, the method is a method of determining efficacy. In some embodiments, the method is a method of determining side effects. In some embodiments, the method is a method of determining dose. In some embodiments, the dose is a therapeutically effective dose. In some embodiments, the contacting is with a therapeutically effective dose.

In some embodiments, the method further comprises testing a physiological output of the cardiac organoid after the contacting or exposing. In some embodiments, the method further comprises measuring at least one parameter of the cardiac organoid after the contacting or exposing. In some embodiments, the parameter is an output. In some embodiments, the parameter is electro-mitochondrial synchronization. In some embodiments, the output is a mechanical output. In some embodiments, the output is an electrical output. In some embodiments, the output is beat rate. In some embodiments, the output is respiration. In some embodiments, the output is oxidative phosphorylation. In some embodiments, the output is mitochondrial maximal capacity. In some embodiments, the output is contraction rhythm. In some embodiments, the rhythm is sinus rhythm. In some embodiments, the output is gene expression. In some embodiments, the output is protein expression.

In some embodiments, the method further comprises comparing the physiological output to an output measured before the contacting or exposing. In some embodiments, the testing comprises the comparing. In some embodiments, an increase indicates effectiveness of the agent or compound. In some embodiments, a decrease indicates effectiveness of the agent or compound. In some embodiments, an increase indicates a side effect of the agent or compound. In some embodiments, a decrease indicates a side effect of the agent or compound.

In some embodiments, the method comprises testing a physiological output of the cardiac organoid before the contacting or exposing. In some embodiments, the method comprises testing a physiological output of the cardiac organoid after the contacting. In some embodiments, the method comprises testing a physiological output of the cardiac organoid before the contacting, testing a physiological output of the cardiac organoid after the contacting, and comparing both testings.

In some embodiments, the testing is or comprises testing for a negative cardiac side effect. In some embodiments, the side effect is a negative side effect. In some embodiments, the negative side effect is a negative cardiac side effect. In some embodiments, a negative cardiac side effect is electro-mitochondrial desynchronization. In some embodiments, a negative cardiac side effect is arrhythmia. In some embodiments, a negative cardiac side effect comprises arrhythmia. As used herein, the term “arrhythmia” refers or encompasses any abnormal or irregular rate or rhythm of a heartbeat. In some embodiments, arrhythmia is selected from: extra beats, supraventricular tachycardias, ventricular arrhythmias, or bradyarrhythmias. In some embodiments, extra beats comprise any one of: premature atrial contractions, premature ventricular contractions, and premature junctional contractions. In some embodiments, supraventricular tachycardias comprise any one of: atrial fibrillation, atrial flutter, and paroxysmal supraventricular tachycardia. In some embodiments, ventricular arrhythmias comprise any one of: ventricular fibrillation and ventricular tachycardia. In some embodiments, bradyarrhythmias comprise any one of: sinus node dysfunction-induced bradyarrhythmias and atrioventricular conduction disturbances-induced bradyarrhythmias. In some embodiments, arrhythmia is therapeutic induced arrhythmia. In some embodiments, the therapeutic is an anticancer therapeutic. In some embodiments, an anticancer therapeutic is a cancer treatment. In some embodiments, arrhythmia is cancer treatment induced arrhythmia (CTIA). In some embodiments, the cancer treatment is doxorubicin. In some embodiments, the cancer treatment is selected from the anticancer drugs provided in Table 1.

Therapeutic agents or conditions that induce cardiac side effects are well known in the art and the subject may be taking any such therapeutic agent or exposed to any such conditions. In some embodiments, the subject is taking a therapeutic agent that induces a cardiac side effect. In some embodiments, the subject is exposed to a condition that induces a cardiac side effect. In some embodiments, the testing is testing an agent with a cardiac side effect or known to produce a cardiac side effect. In some embodiments, the testing is testing a condition known to produce a cardiac side effect. Examples of agents with cardiac side effects can be found for example in Mamoshina et al., 2021 “Toward a broader view of mechanisms of drug cardiotoxicity”, Cell Reports Medicine, March 16; 2(3):100216, herein incorporated by reference in its entirety. In some embodiments, the therapeutic is selected from those provided in Table 1. In some embodiments, the therapeutic is an antineoplastic agent. In some embodiments, antineoplastic agent is an anticancer agent. In some embodiments, the therapeutic is an anti-inflammatory agent. In some embodiments, the therapeutic is a central nervous system agent. In some embodiments, the therapeutic agent is a gastrointestinal agent. In some embodiments, the therapeutic is a genitourinary system agent. In some embodiments, the therapeutic is an antiallergic agent. In some embodiments, the therapeutic is an anti-infective agent. In some embodiments, the therapeutic is a cardiovascular agent. Examples of these agents with cardiac side effects can be found in Table 1.

TABLE 1
Therapeutic agents with cardiac side effects

| | |
|---|---|
| Drug | Side effect |

Antineoplastic agents

| | |
|---|---|
| 5-fluorouracil | arrhythmias, myocardial ischemia, heart failure |
| Arsenic trioxide | QT prolongation, tachycardia |
| Bevacizumab | heart failure |
| Bortezomib | heart failure, arrhythmia |
| Cisplatin | arrhythmias, myocardial ischemia, heart failure |
| Cytarabine | bradycardia, heart failure, ischemia |
| Daunorubicin | arrhythmias, heart failure |
| Dasatinib | heart failure |
| Docetaxel | bradycardia, myocardial ischemia, heart failure |
| Doxorubicin | arrhythmias, heart failure |
| Idarubicin | arrhythmias, heart failure |
| Imatinib | systolic heart failure, heart failure, left ventricular dysfunction |
| Ipilimumab | lethal myocarditis |
| Lapatinib | left ventricular ejection fraction, congestive heart failure |
| Nilotinib | myocardial ischemia |
| Nivolumab | lethal myocarditis |
| Paclitaxel | bradycardia, myocardial ischemia, heart failure |
| Romidepsin | QT prolongation, myocardial infarction |
| Sorafenib | heart failure, myocardial ischemia, QT prolongation |
| Sunitinib | long QT, left ejection fraction, myocardial infarction |
| Trastuzumab | heart failure, tachycardia |
| Vandetanib | long QT |
| Vinblastine | myocardial ischemia, heart failure |

Anti-inflamatory agents

| | |
|---|---|
| Diclofenac | myocardial infarction |
| Etoricoxib | thrombotic events |
| Ibuprofen | myocardial infarction, hypertension |
| Indomethacin | myocardial infarction |
| Naproxen | myocardial infarction |
| Rofecoxib | myocardial infarction |

Central nervous system agents

| | |
|---|---|
| Benfluorex | valvular heart disease |
| Bupivacaine | ventricular arrhythmias, myocardial depression |
| Chlorphentermine | pulmonary heart disease |
| Clozapine | myocarditis, cardiomyopathy |
| Cocaine | left ventricular hypertrophy, arrhythmias |
| Dexfenfluramine | valvular heart disease |
| Ergotamine | valvular heart disease |
| Fenfluramine | valvular heart disease |
| Fluoxetine | Bradycardia |
| Haloperidol | QT prolongation, torsade de pointes (TdP), sudden cardiac death |
| Levomethadyl acetate | QT prolongation, TdP |
| Lidocaine | bradycardia, cardiac arrest |
| Methysergide | valvular heart disease |
| Pergolide | valvular heart disease |
| Phentermine | valvular heart disease |
| Propoxyphene | QT prolongation, TdP, |
| Sertindole | QT prolongation, TdP, sudden cardiac death |
| Sibutramine | myocardial infarction |
| Thioridazine | QT prolongation, TdP, sudden cardiac death |
| Venlafaxine | QT prolongation, arrhythmias |
| Ziprasidone | QT prolongation, TdP, sudden cardiac death |

Gastrointestinal agents

| | |
|---|---|
| Cispapride | ventricular arrhythmia, QT prolongation, TdP, cardiac arrest |
| Loperamide | cardiac arrest, QT prolongation, ventricular tachycardia, TdP |
| Omeprazole | acute myocardial infarction, heart failure |
| Tegaserod | Ischemia |

Genitourinary System agent

| | |
|---|---|
| Terodiline | ventricular tachycardia, cardiac death |

Antiallergic agents

| | |
|---|---|
| Astemizole | long QT syndrome, TdP |
| Diphenhydramine | QT prolongation |
| Terfenadine | QT prolongation, TdP |

Anti-infective agents

| | |
|---|---|
| Azidothymidine | dilated cardiomyopathy |
| Azithromycin | QT prolongation, TdP, cardiac death |
| Clarithromycin | QT prolongation, myocardial infarction, arrhythmias, cardiac death |
| Erthyromycin | QT prolongation, ventricular tachycardia, TdP, ventricular fibrillation |
| Grepafloxacin | QT prolongation |
| Sofobuvir | Bradycardia |
| Sparfloxacin | QT prolongation |
| Pentamidine | QT prolongation, arrhythmias |

Cardiovascular agents

| | |
|---|---|
| Buflomedil | QT prolongation, cardiac arrest |
| Dofetilide | QT prolongation, TdP |
| Encainide | QT prolongation, TdP |
| Lidoflazine | QT prolongation |
| Mibefradil | QT prolongation |
| Orciprenaline | tachycardia, palpitations |
| Prenylamine | QT prolongation, sudden cardiac death, ventricular tachycardia, TdP |
| Probucol | QT prolongation, arrhythmias |

Other agents

| | |
|---|---|
| Alogliptin | heart failure |
| Clobutinol | QT prolongation |
| Rosiglitazone | heart failure |
| Saxagliptin | heart failure |

In some embodiments, the therapeutic is selected from 5-fluorouracil, Arsenic trioxide, Bevacizumab, Bortezomib, Cisplatin, Cytarabine, Daunorubicin, Dasatinib, Docetaxel, Doxorubicin, Idarubicin, Imatinib, Ipilimumab, Lapatinib, Nilotinib, Nivolumab, Paclitaxel, Romidepsin, Sorafenib, Sunitinib, Trastuzumab, Vandetanib, Vinblastine, Diclofenac, Etoricoxib, Tbuprofen, Indomethacin, Naproxen, Rofecoxib, Central nervous system agents, Benfluorex, Bupivacaine, Chlorphentermine, Clozapine, Cocaine, Dexfenfluramine, Ergotamine, Fenfluramine, Fluoxetine, Haloperidol, Levomethadyl acetate, Lidocaine, Methysergide, Pergolide, Phentermine, Propoxyphene, Sertindole, Sibutramine, Thioridazine, Venlafaxine, Ziprasidone, Cispapride, Loperamide, Omeprazole, Tegaserod, Terodiline, Astemizole, Diphenhydramine, Terfenadine, Azidothymidine, Azithromycin, Clarithromycin, Erthyromycin, Grepafloxacin, Sofobuvir, Sparfloxacin, Pentamidine, Buflomedil, Dofetilide, Encainide, Lidoflazine, Mibefradil, Orciprenaline, Prenylamine, Probucol, Alogliptin, Clobutinol, Rosiglitazone, and Saxagliptin. In some embodiments, the therapeutic agent interacts with MCU. In some embodiments, interaction with MCU inhibits MCU activity. In some embodiments, MCU activity is calcium transport. In some embodiments, the therapeutic agent interacts with a calcium channel. In some embodiments, the therapeutic agent that interacts with MCU is selected from Sorafenib, Sunitinib, Vandetanib, Bupivacaine, Cocaine, Fluoxetine, Haloperidol, Levomethadyl, Propoxyphene, Sertindole, Thioridazine, Venlafaxine, Ziprasidone, Cisapride, Loperamide, Terodiline, Astemizole, Diphenhydramine, Terfenadine, Azithromycin, Mitoxantrone, Clarithromycin, Erythromycin, Grepafloxacin, Sofosbuvir, Sparfloxacin, Pentamidine, Buflomedil, Dofetilide, Encainide, Lidoflazine, Mibefradil, Orciprenaline, Prenylamine, Probucol, and Clobutinol.

In some embodiments, the cardiac side effect is electro-mitochondrial desynchronization. In some embodiments, the cardiac side effect is arrhythmia. In some embodiments, arrhythmia is ventricular arrhythmia. In some embodiments, arrhythmia is supraventricular arrhythmia. In some embodiments, the arrhythmia is inherited arrhythmia. In some embodiments, the arrhythmia is bradycardia. In some embodiments, the arrhythmia is tachycardia. In some embodiments, the arrhythmia is valvular atrial fibrillation. In some embodiments, the cardiac side effect is ischemia. In some embodiments, ischemia is myocardial ischemia. In some embodiments, the cardiac side effect is heart failure. In some embodiments, heart failure is systolic heart failure. In some embodiments, the cardiac side effect is QT prolongation. In some embodiments, the cardiac side effect is tachycardia. In some embodiments, the tachycardia is ventricular tachycardia. In some embodiments, the cardiac side effect is bradycardia. In some embodiments, the cardiac side effect is left ventricular dysfunction. In some embodiments, the cardiac side effect is myocarditis. In some embodiments, myocarditis is lethal myocarditis. In some embodiments, the cardiac side effect is myocardial infarction. In some embodiments, the cardiac side effect is long QT. In some embodiments, the cardiac side effect is left ejection fraction. In some embodiments, the cardiac side effect is a thrombotic event. In some embodiments, the cardiac side effect is hypertension. In some embodiments, the cardiac side effect is valvular heart disease. In some embodiments, the cardiac side effect is myocardial depression. In some embodiments, the cardiac side effect is pulmonary heart disease. In some embodiments, the cardiac side effect is myocarditis. In some embodiments, the cardiac side effect is cardiomyopathy. In some embodiments, the cardiomyopathy is dilated cardiomyopathy. In some embodiments, the cardiac side effect is left ventricular hypertrophy. In some embodiments, the cardiac side effect is torsade de pointes (TdP). In some embodiments, the cardiac side effect is sudden cardiac death. In some embodiments, the cardiac side effect is cardiac arrest. In some embodiments, the cardiac side effect is long QT syndrome. In some embodiments, the cardiac side effect is ventricular fibrillation. In some embodiments, the cardiac side effect is palpitations.

In some embodiments, the therapeutic agent is a drug. In some embodiments, the therapeutic agent is a small molecule. In some embodiments, the therapeutic agent is a biologic. In some embodiments, the therapeutic agent is a cardiac therapeutic agent. In some embodiments, the therapeutic agent is a non-cardiac therapeutic agent. In some embodiments, the therapeutic agent is a therapeutic agent suspected of causing a cardiac side effect. In some embodiments, the therapeutic agent is an agent that is administered systemically. In some embodiments, the therapeutic agent is an agent that is formulated for systemic administration. In some embodiments, the therapeutic agent is an agent that is being considered for systemic administration.

In some embodiments, the therapeutic agent is an anticancer therapeutic agent. In some embodiments, the anticancer agent is chemotherapeutic. In some embodiments, the chemotherapeutic is mitoxantrone. In some embodiments, the therapeutic agent is suitable for treating, preventing, or ameliorating a heart disease or a condition or a symptom associated therewith. In some embodiments, the therapeutic is a calcium signaling targeting agent. In some embodiments, calcium signaling targeting agent comprises any compound capable of modulating calcium transport, mobilization, exchange, or any combination thereof, in a cell. In some embodiments, modulating comprises increasing or enhancing. In some embodiments, modulating comprises reducing or inhibiting.

In some embodiments, the calcium signaling targeting agent reduces flux of calcium across the mitochondrial membrane. In some embodiments, the calcium signaling agent reduces the frequency of calcium oscillation across a mitochondrial membrane. In some embodiments, the calcium signaling agent reduces the rate of calcium flux, oscillation, mobilization, transfer, or any combination thereof, across a mitochondrial membrane. In some embodiments, a mitochondrial membrane comprises the inner mitochondrial membrane, the outer mitochondrial membrane, or both.

In some embodiments, the anticancer agent is or comprises a calcium signaling targeting agent. In some embodiments, the agent comprises or is a calcium channel blocker. In some embodiments, the anticancer agent induces, stimulates, enhances, promotes, or any combination thereof arrhythmia. In some embodiments, the anticancer agent is suspected of inducing CTIA. Types of calcium channel blockers would be apparent to an ordinarily skilled physician. Non-limiting examples of such calcium channel blockers include, but are not limited to, Amlodipine (Norvasc), Diltiazem (Cardizem, Tiazac, others), Felodipine, Isradipine, Nicardipine, Nifedipine (Procardia), Nisoldipine (Sular), and Verapamil (Calan SR, Verelan), to name a few. In some embodiments, increasing mitochondrial calcium comprises increasing mitochondrial activity. In some embodiments, mitochondrial activity comprises oxidative phosphorylation.

In some embodiments, the method further comprises inducing a cardiac deficiency, condition or disease, or a simulated condition thereof, in the cardiac organoid before the contacting. In some embodiments, inducing comprises genetic modification of cells of the organoid. In some embodiments, the therapeutic agent is a designed to treat the induced deficiency, condition or disease, or a simulating condition thereof. In some embodiments, the method is a method of testing efficacy of the therapeutic agent. It will be understood by a skilled artisan that a condition or disease can be induced in the organoid in order to test an agent that is designed to treat that condition or disease. In some embodiments, the condition or disease is or comprises arrhythmia. In some embodiments, arrhythmia is induced by contacting the organoid with mitoxantrone.

According to another aspect, there is provided a method for treating arrhythmia in a subject.

According to another aspect, there is provided a method for treating a disease or disorder in a subject.

In some embodiments, the disease or disorder is characterized by electro-mitochondrial desynchronization. In some embodiments, a symptom of the disease or disorder is caused by electro-mitochondrial desynchronization. As described hereinbelow for the first time, mitochondrial function and electrical activity of a tissue can become uncoupled. This desynchronization leads to impaired cellular function. While it is well known that impaired mitochondrial function (e.g., energy output) can lead to diseases and disorders and it is known that ion levels have an impact on mitochondrial function, it was heretofore not known that the ion fluxes into the cell and into the mitochondria must be kept in lockstep to coordinate oxygen consumption with other cellular functions and maintain cellular homeostasis. Thus, impairment of normal biological function can occur even when energy output from the mitochondria and the electrical activity in the mitochondrial and tissue both individually appear normal, but when the two are no longer synchronized.

In some embodiments, the disease is selected from the group consisting of: arrhythmia, cardiomyopathy, seizures, epilepsy, motor neuron spasms, muscle weakness, muscular atrophy, a channelopathy, Catecholaminergic polymorphic ventricular tachycardia (CPVT), myopathy with extrapyramidal signs (MPXPS), Alzheimer's disease, Huntington's disease, Parkinson's disease, amyotrophic lateral sclerosis (AML), hereditary spastic paraplegia, ischemia-reperfusion injury, ischemic heart disease, rare mitochondrial encephalomyopathy, Sagittal Sinus Thrombosis, Intracranial Sinus Thrombosis, Stormorken Syndrome, Generalized Epilepsy With Febrile Seizures Plus, Optic Atrophy 3, Autosomal Dominant, Generalized Epilepsy With Febrile Seizures Plus, Type 6, Palmoplantar Keratoderma, Nonepidermolytic, and Eastern Equine Encephalitis. It will be understood by a skilled artisan that the above listed diseases/conditions are examples of diseases/conditions that can be characterized by electro-mitochondrial desynchronization. However, not all cases of these diseases/conditions are so characterized (i.e., there is not desynchronization components to how the disease/condition manifests in some subjects) and so a skilled artisan will need to determine if desynchronization is present in any given subject. In some embodiments, the arrhythmia is cancer treatment induced arrhythmia (CTIA). In some embodiments, the cancer treatment is doxorubicin. In some embodiments, the cancer treatment is selected from the anticancer agents provided in Table 1. Channelopathies are well known in the art and are summarized for example in Kim, 2014, “Channelopathies”, Korean J. Pediatr.; 57(1):1-18, herein incorporated by reference in its entirety.

In some embodiments, the method further comprises confirming electro-mitochondrial desynchronization. In some embodiments, the method further comprises confirming the disease or disorder is characterized by electro-mitochondrial desynchronization. In some embodiments, the method further comprises confirming electro-mitochondrial desynchronization in the subject. In some embodiments, the method further comprises confirming the disease or disorder manifests by electro-mitochondrial desynchronization in the subject. It will be understood by a skilled artisan that while many diseases can have a component, symptom, or cause that is electro-mitochondrial in nature not every manifestation of the disease will have it. Thus, the method may include determining that in this particular subject suffering from the disease electro-mitochondrial desynchronization is present. In some embodiments, the confirming is in the subject. In some embodiments, the confirming is a tissue of the subject. In some embodiments, the tissue is an electrical tissue. Examples of electrical tissues include, but are not limited to, neurons and cardiac tissue. In some embodiments, the tissue is cardiac tissue. In some embodiments, the tissue is neuronal tissue. In some embodiments, the tissue is neurons. In some embodiments, the tissue is central nervous system tissue. In some embodiments, the tissue is brain. In some embodiments, the tissue is peripheral nervous system tissue. In some embodiments, the tissue is diseased tissue. In some embodiments, the tissue is tissue of the disease or disorder. In some embodiments, the tissue is from the subject. In some embodiments, the tissue is derived from cells from the subject. In some embodiments, derived from comprises expanded from. In some embodiments, derived from comprises differentiated from. In some embodiments, the tissue is a biopsy. In some embodiments the tissue is a tissue contacted with a drug or agent. In some embodiments, the subject is receiving the drug or agent. In some embodiments, the method further comprises selecting a subject suffering from the disease or disorder. In some embodiments, the method further comprises determining a symptom of a disease or disorder of the subject is caused by electro-mitochondrial desynchronization.

In some embodiments, desynchronization is desynchronization of mitochondrial function and electrical activity. In some embodiments, electrical activity is electrical activity in the mitochondria. In some embodiments, electrical activity is electrical activity in the tissue. In some embodiments, the tissue is the tissue comprising the mitochondria. Any method for determining both electrical activity and mitochondrial function at the same time can be used to assess synchronization. In some embodiments, the method of confirming/determining electro-mitochondrial desynchronization is in vitro. In some embodiments, the method of confirming/determining electro-mitochondrial desynchronization is in vivo. In some embodiments, the method of confirming/determining electro-mitochondrial desynchronization is ex vivo. In some embodiments, the method of confirming/determining electro-mitochondrial desynchronization is a method of the invention. In some embodiments, the method of confirming/determining electro-mitochondrial desynchronization is a method described hereinbelow. In some embodiments, the method of confirming/determining electro-mitochondrial desynchronization comprises evaluating tissue in a system of the invention. In some embodiments, the method of confirming/determining electro-mitochondrial desynchronization comprises using a system of the invention to measure electro-mitochondrial desynchronization. In some embodiments, the confirming/determining comprises measuring mitochondrial activity. In some embodiments, mitochondrial activity comprises oxygen consumption. In some embodiments, mitochondrial activity comprises mitochondrial calcium levels. In some embodiments, the confirming/determining comprises measuring field potential in the tissue and contraction in the tissue. In some embodiments, the confirming/determining comprises measuring field potential in the tissue and oxygen consumption in the tissue. In some embodiments, the confirming/determining comprises measuring field potential in the tissue and calcium levels in the tissue. In some embodiments, the confirming/determining comprises measuring oxygen consumption in the tissue and contraction in the tissue. In some embodiments, the confirming/determining comprises measuring calcium levels in the tissue and contraction in the tissue. In some embodiments, the confirming/determining comprises measuring field potential in the tissue, oxygen consumption in the tissue and contraction in the tissue. In some embodiments, the confirming/determining comprises measuring field potential in the tissue, calcium levels in the tissue and contraction in the tissue. In some embodiments, the method of confirming/determining electro-mitochondrial desynchronization comprises using a system of the invention to measure oxygen consumption in the tissue and calcium levels in the tissue. In some embodiments, the method of confirming/determining electro-mitochondrial desynchronization comprises using a system of the invention to measure contraction in the tissue and oxygen consumption in the tissue. In some embodiments, measure is simultaneously measure

Beyond the methods provided herein to confirm/determine electro-mitochondrial desynchronization, other methods known in the art can be employed to this purpose. For example, typical and well-known method of measuring electrical output can be used. These include EKG, EMG, EEG, ECG and EOG. Desynchronization can be seen in these measures alone and will manifest as late potentials, reduced R waves, increased R/R ratios and other abnormalities. Additional methods such as those described in Saminathan et al., 2021, “A DNA-based voltmeter for organelles”, Nature Nanotechnology, 16, 96-103, herein incorporated by reference in its entirety, may also be used. Mitochondrial function can also be evaluated in tissue using functional stains for oxygen or calcium, high resolution microscopy or in vivo calcium measurement. In particular, mitochondrial calcium levels can be evaluated by a mitochondrial calcium selective dye such as Rhod-2AM or can be tracked by NMR/IR scanning. Generally, methods of measuring mitochondrial calcium in vivo can be found for example in Pozzan and Rudolf, 2009, “Measurement of mitochondrial calcium in vivo”, Biochim Biophys Acta., November; 1787(11):1317-23, and Serrat et al., 2022, “Imaging mitochondrial calcium dynamics in the central nervous system”, J. Neuroscience Methods, vol 373, May; 209560, herein incorporated by reference in their entirety. Simultaneous methods of measurement such as measuring oxygen consumption by protoporphyrin IX-triplet state lifetime technique, NMR or other optical methods and electrical activity such as by EEG/EMG/ECG is known and can be seen for example is Campbell and Marcinek, 2017, “Evaluation of in vivo mitochondrial bioenergetics in skeletal muscle using NMR and optical methods”, Biochim Biophys Acta, April; 1862(4):716-724, herein incorporated by reference in its entirety. Genetic screens of mitochondrial genes and the discovery of calcium channel mutations may indicate a desynchrony. Similarly, use of a drug with a known desynchronizing effect, such as a cancer treatment that produces arrhythmia (CTIA), is another indication that the condition is characterized by desynchronization.

In some embodiments, confirming/determining comprises determining mitochondrial calcium concentration in a sample obtained from the subject. In some embodiments, determining is measuring. In some embodiments, the sample is a fluid sample. In some embodiments, the sample comprises cells. In some embodiments, the cells are cardiac cells. In some embodiments, the cells are disease cells. In some embodiments, the sample is a cell-free sample. In some embodiments, the fluid is a bodily fluid. In some embodiments, the fluid is selected from blood, serum, plasma, tumor fluid, gastric fluid, intestinal fluid, saliva, bile, tumor fluid, breast milk, urine, interstitial fluid, cerebral spinal fluid and stool. In some embodiments, the fluid is blood. In some embodiments, the blood is peripheral blood. In some embodiments, calcium concentration beyond a predetermined threshold is indicative of desynchronization. In some embodiments, the predetermined threshold is the calcium concentration in a subject that does not suffer from electro-mitochondrial desynchronization. In some embodiments, the predetermined threshold is the calcium concentration in a healthy subject. In some embodiments, the predetermined threshold is the calcium concentration in a subject that suffers from the disease or disorder but that is not characterized by electro-mitochondrial desynchronization.

In some embodiments, beyond is above. In some embodiments, beyond is below. It will be understood by a skilled artisan that if the threshold is a maximum allowed value then beyond it will be above it, wherein if a threshold is a minimum allowed value beyond it will be below it. In some embodiments, beyond the threshold is significantly beyond. In some embodiments, significantly is statistically significantly. In some embodiments, beyond is at least 5, 10, 15, 20, 25, 30, 35, 40, 45 or 50% beyond the threshold. Each possibility represents a separate embodiment of the invention. In some embodiments, beyond is at least 10% beyond. In some embodiments, beyond is at least 30% beyond.

In some embodiments, confirming/determining comprises observing in the subject arrhythmic or proarrhythmic symptoms. In some embodiments, the symptoms are arrythmia. In some embodiments, the symptoms to do not respond to antiarrhythmic treatment. In some embodiments, presence of the symptoms indicates desynchronization. In some embodiments, the antiarrhythmic treatment targets electrical activity through membrane channels. In some embodiments, the membrane channels are not calcium channels. In some embodiments, the membrane channel is not a calcium channel other than a non-dihydropyridine calcium channel. In some embodiments, the calcium channel is a non-mitochondrial calcium channel. In some embodiments, the calcium channel is a mitochondrial calcium channel. It will be understood by a skilled artisan that if a calcium channel blocker is not effective then activation of the calcium channel is required as described in the method provided herein. In some embodiments, the antiarrhythmic treatment is a sodium channel blocker. Sodium channels blockers prevent sodium from entering cells and thus can slow electrical impulses in heart muscle. Example of sodium channel blockers include, but are not limited to: disopyramide, flecainide, mexiletine, propafenone, and quinidine. In some embodiments, the antiarrhythmic treatment is a beta blocker. Beta blockers slow heart rate, often by blocking hormones such as adrenaline. Example of beta blockers include, but are not limited to: acebutolol, atenolol, bisoprolol, metoprolol, nadolol, and propranolol. In some embodiments, the antiarrhythmic treatment is a potassium channel blocker. Potassium channel blockers prevent potassium from entering cells and can thus slow electrical impulses in the heart. Example of potassium channel blockers include, but are not limited to: amiodarone, bretylium, dofetilide, dronedarone, ibutilide and sotalol. In some embodiments, the antiarrhythmic treatment is a non-dihydropyridine calcium channel blocker. Non-dihydropyridine calcium channel blockers prevent calcium from entering heart cells, which can decrease heart rate and contractions. Examples of non-dihydropyridine calcium channel blockers include, but are not limited to diltiazem and verapamil. In some embodiments, the antiarrhythmic treatment is adenosine. Adenosine blocks/slows electrical impulses at the atrioventricular node. In some embodiments, the antiarrhythmic treatment is a digoxin. Digoxin slows the heart rate and increases the contractility of the heart.

In some embodiments, confirming/determining comprises performing an electrical test selected from EKG, EEG and EMG. In some embodiments, confirming/determining comprises performing an electrocardiogram (EKG or ECG). In some embodiments, confirming/determining comprises performing an electroencephalogram (EEG). In some embodiments, confirming/determining comprises performing an electromyogram (EMG). In some embodiments, confirming/determining comprises performing an electrooculogram (EOG). In some embodiments, an abnormal electrical readout indicates desynchronization. In some embodiments, an abnormal readout is as compared to a readout from a healthy subject. In some embodiments, an abnormal readout is as compared to a readout from a subject suffering from the disease or disorder but which is not characterized by electro-mitochondrial synchronization. In some embodiments, an abnormal readout comprises late potentials. In some embodiments, an abnormal readout comprises reduced R waves. In some embodiments, an abnormal readout comprises increased R/R ratio.

In some embodiments, confirming/determining comprises confirming exposure to an agent that is known to produce electro-mitochondrial desynchronization. In some embodiments, the agent is a drug. In some embodiments, the agent is a chemical. In some embodiments, the agent is an agricultural chemical. In some embodiments, the chemical is a solvent. In some embodiments, the chemical is a pesticide. In some embodiments, the agent is selected from those provided in Table 1. In some embodiments, a chemical agent is selected from toluene, trichloroethane, xylene, heptanes, hexane, ethyl ether trichloroethylene, and trichlorotrifluoroethane. Other examples of agents include, but are not limited to carbon monoxide, carbon disulfide, pesticides, methane-derived halogenated hydrocarbons, caffeine, bisphenol A, organic nitrates, arsenic, cadmium, cobalt, organic solvents, and metals.

According to another aspect, there is provided a method for treating a cell proliferation related disease in a subject.

According to another aspect, there is provided a method for treating a disease in a subject, comprising administering a first agent and a second agent, wherein the disease is treatable by the first agent.

According to another aspect, there is provided a method for treating a cell proliferation related disease in a subject, comprising administering a first agent and a second agent.

In some embodiments, the first agent produces a cardiac side effect. In some embodiments, the side effect is a detrimental side effect. In some embodiments, the first agent is suspected of producing or is likely to produce a cardiac side effect. In some embodiments, the disease is cancer and the first agent is an antineoplastic agent. In some embodiments, the disease is an inflammatory disease and the first agent is an anti-inflammatory agent. In some embodiments, the disease is a disease of the central nervous system (CNS) and the first agent is a CNS agent. In some embodiments, the CNS disease is a brain disease. In some embodiments, the CNS disease is a neuronal disease. In some embodiments, the disease is a gastrointestinal disease and the first agent is a gastrointestinal agent. In some embodiments, the disease is a genital or urinary disease and the first agent is a genitourinary agent. In some embodiments, the disease is an allergic reaction and the first agent is an antiallergic agent. In some embodiments, the disease is an infection and the first agent is an anti-infective agent. In some embodiments, an anti-infective agent is an antibiotic. In some embodiments, anti-infective agent is an antiviral agent. In some embodiments, an anti-infective agent is a vaccine. In some embodiments, the disease is a cardiovascular disease and the first agent is a cardiovascular agent. In some embodiments, the first agent is selected from the agents provided in Table 1.

Examples of inflammatory diseases include, but are not limited to autoimmune disease, rheumatoid arthritis, inflammatory bowel syndrome (IBS), inflammatory bowel disease (IBD), colitis, Crohn's disease, ankylosing spondylitis, antiphospholipid antibody syndrome, gout, myositis, scleroderma, lupus, Sjogren's syndrome and vasculitis to name but a few. Examples of CNS diseases include, but are not limited to Alzheimer's disease, Parkinson's disease, amyotrophic lateral sclerosis (ALS), muscular dystrophy, palsy, epilepsy, multiple sclerosis (MS), neurofibromatosis and sciatica to name but a few. Examples of gastrointestinal diseases include, but are not limited to IBS, IBD, acid reflux, gird, hemorrhoids, intestinal cancer, colon polyps and diverticular disease to name but a few. Examples of genital or urinary diseases include but are not limited to urinary tract infection, testicular cancer, renal failure, endometrial ablation, erectile dysfunction, incontinence, kidney stones, prostate cancer, sickle cell nephropathy and yeast infections to name but a few. Examples of allergic reactions include but are not limited to food allergies, asthma, environmental allergies, and drug allergies to name but a few. Examples of infections include, but are not limited to bacterial infections, viral infections, and parasitic infections. Examples of cardiovascular disease include, but are not limited to heart attack, stroke, heart failure, arrhythmia, and palpitations to name but a few.

In some embodiments, the method comprises administering to the subject a therapeutically effective amount of an agent that modulates mitochondrial calcium concentration. In some embodiments, the treating comprises administering to the subject a therapeutically effective amount of an agent that modulates mitochondrial calcium concentration. In some embodiments, the method comprises administering to the subject a therapeutically effective amount of an agent that modulates mitochondrial calcium channel activity. In some embodiments, the treating comprises administering to the subject a therapeutically effective amount of an agent that modulates mitochondrial calcium channel activity. In some embodiments, the method comprises administering to the subject a therapeutically effective amount of an agent that modulates mitochondrial calcium uniporter (MCU) activity. In some embodiments, the treating comprises administering to the subject a therapeutically effective amount of an agent that modulates MCU activity. In some embodiments, the mitochondrial calcium channel is MCU. In some embodiments, the mitochondrial calcium channel is Voltage-dependent anion channel (VDAC). In some embodiments, a mitochondrial calcium channel is not a non-dihydropyridine calcium channel. Examples of mitochondrial calcium channels include, but are not limited to MCU and VDAC. Example of other mitochondrial calcium channels include permeability mode of uptake (RaM), ryanosine receptor (mRyR or RyRI), the mitochondrial rapid exchanger (mHCX), DAG activated+tramsition pore (mPTP), Na+/Ca2+ exchanger (mNCX), and H+/Ca2 cation channels (DCC) which are well known in the art. Other examples can be found for example in Malli et al., 2014, “Mitochondrial Ca2+ channels: great unknowns with important functions”, FEBS Lett. May 17; 584(10): 1942-1947, herein incorporated by reference in its entirety. In some embodiments, the increase is in a tissue. In some embodiments, the tissue is a tissue of the subject. In some embodiments, the increase is in a heart tissue. In some embodiments, the heart tissue is a heart tissue of the subject. In some embodiments, the modulating is modulating mitochondrial activity. In some embodiments the modulation in activity is in the tissue.

In some embodiments, modulating MCU activity comprises inhibiting interaction of a drug with MCU. In some embodiments, interaction is binding. In some embodiments, the drug inhibits MCU activity. In some embodiments, the drug activates MCU activity. In some embodiments, interaction of the drug with MCU inhibit or activates MCU activity. In some embodiments, the agent inhibits or induces interaction of a drug with MCU. In some embodiments, inhibits is blocks. In some embodiments, induces is triggers. In some embodiments, agent competes with MCU for binding to the drug. In some embodiments, the agent is a peptide that binds the drug. In some embodiments, the agent is a small molecule that binds the drug. In some embodiments, an agent that interacts with MCU is selected from Sorafenib, Sunitinib, Vandetanib, Bupivacaine, Cocaine, Fluoxetine, Haloperidol, Levomethadyl, Propoxyphene, Sertindole, Thioridazine, Venlafaxine, Ziprasidone, Cisapride, Loperamide, Terodiline, Astemizole, Diphenhydramine, Terfenadine, Azithromycin, Mitoxantrone, Clarithromycin, Erythromycin, Grepafloxacin, Sofosbuvir, Sparfloxacin, Pentamidine, Buflomedil, Dofetilide, Encainide, Lidoflazine, Mibefradil, Orciprenaline, Prenylamine, Probucol, and Clobutinol. In some embodiments, the administering of the agent treats arrhythmia. In some embodiments, the agent is a second agent.

In some embodiments, modulating comprises administering an agent selected from: metformin, kaempferol, spermine, A-769662, AICAR, IND 1316, PF 06409577, ZLN 024, Erastin, Honokiol, Ezetimibe, Disulfiram, Efsevin and spermidine. In some embodiments, modulating comprises administering an agent selected from: metformin, spermine, A-769662, AICAR, IND 1316, PF 06409577, ZLN 024, and spermidine. In some embodiments, the agent is metformin. In some embodiments, the agent is selected from metformin, spermidine and spermine.

In some embodiments, the method comprises administering to the subject a therapeutically effective amount of an agent capable of modulating mitochondrial calcium concentration in a heart tissue of the subject, thereby treating a cardiac disease or disorder characterized by electro-mitochondrial desynchronization. In some embodiments, the method comprises administering to the subject a therapeutically effective amount of an agent capable of modulating mitochondrial calcium concentration in a neuronal tissue of the subject, thereby treating a nervous disease or disorder characterized by electro-mitochondrial desynchronization. In some embodiments, the method comprises administering to the subject a therapeutically effective amount of an agent capable of modulating mitochondrial calcium concentration in a heart tissue of the subject, thereby treating arrhythmia. In some embodiments, the method comprises administering to the subject a therapeutically effective amount of an agent capable of modulating mitochondrial calcium channel activity in a heart tissue of the subject, thereby treating a cardiac disease or disorder characterized by electro-mitochondrial desynchronization. In some embodiments, the method comprises administering to the subject a therapeutically effective amount of an agent capable of modulating mitochondrial calcium channel activity in a neuronal tissue of the subject, thereby treating a nervous disease or disorder characterized by electro-mitochondrial desynchronization. In some embodiments, the method comprises administering to the subject a therapeutically effective amount of an agent capable of modulating mitochondrial calcium channel activity in a heart tissue of the subject, thereby treating arrhythmia. In some embodiments, the agent is a second agent.

In some embodiments, modulating is increasing. In some embodiments, modulating is decreasing. In some embodiments, increasing is increasing to above a predetermined threshold. In some embodiments, decreasing is decreasing to below a predetermined threshold. In some embodiments, increasing is producing a statistically significant increase. In some embodiments, decreasing is producing a statistically significant decrease. In some embodiments, the predetermined threshold is the levels or activity in a healthy subject. In some embodiments, the desynchronization comprises decreased mitochondrial calcium concentration and the modulating is increasing. In some embodiments, the desynchronization comprises decreased mitochondrial calcium channel activity and the modulating is increasing. In some embodiments, decreased is significantly decreased. In some embodiments, decreased is as compared to a healthy control. In some embodiments, decreased is as compared to a subject suffering from the disease but not characterized by desynchronization. In some embodiments, the desynchronization comprises increased mitochondrial calcium concentration and the modulating is decreasing. In some embodiments, the desynchronization comprises increased mitochondrial calcium channel activity and the modulating is decreasing. In some embodiments, increased is significantly increased. In some embodiments, increased is as compared to a healthy control. In some embodiments, increased is as compared to a subject suffering from the disease but not characterized by desynchronization.

In some embodiments, the desynchronization comprises increased mitochondrial calcium concentration or increased mitochondrial calcium channel activity and the disease or disorder is selected from: mitochondrial encephalomyopathy, Sagittal Sinus Thrombosis, Intracranial Sinus Thrombosis, Stormorken Syndrome, Generalized Epilepsy With Febrile Seizures Plus, Optic Atrophy 3, Autosomal Dominant, Generalized Epilepsy With Febrile Seizures Plus, Type 6, Palmoplantar Keratoderma, Nonepidermolytic, Eastern Equine Encephalitis and CTIA. In some embodiments, the CTIA is doxorubicin induced arrhythmia. In some embodiments, the desynchronization comprises decreased mitochondrial calcium concentration or increased mitochondrial calcium channel activity and the disease or disorder is selected from: arrhythmia, cardiomyopathy, seizures, epilepsy, motor neuron spasms, muscle weakness, muscular atrophy, a channelopathy, CPVT, MPXPS, Alzheimer's disease, Huntington's disease, Parkinson's disease, AML, hereditary spastic paraplegia, ischemia-reperfusion injury, ischemic heart disease, rare and CTIA.

In some embodiments, the disease or disorder is a genetic disease or disorder. In some embodiments, the genetic disease or disorder induces arrhythmia and/or cardiac disfunction. Example of such genetic diseases include but are not limited to: CPTV and MPXPS. In some embodiments, the disease or disorder is CPVT and the modulating comprises administering an agent selected from Spermine, Spermidine, Metformin, Erastin, A-769662, AICAR, IND 1316, PF 06409577 and ZLN 024. In some embodiments, the genetic disorder or disease induces neurorythmic disorders. Examples of such genetic disease include but are not limited to: rare mitochondrial encephalomyopathy, Sagittal Sinus Thrombosis, Intracranial Sinus Thrombosis, Stormorken Syndrome, Generalized Epilepsy With Febrile Seizures Plus, Optic Atrophy 3, Autosomal Dominant, Generalized Epilepsy With Febrile Seizures Plus, Type 6, Palmoplantar Keratoderma, Nonepidermolytic, and Eastern Equine Encephalitis.

In some embodiments, the disease or disorder is a symptom of a disease or disorder. In some embodiments, the disease or disorder is a cardiac disease or disorder. In some embodiments, a cardiac disease or disorder is selected from an arrhythmia and a cardiomyopathy. In some embodiments, the disease or disorder is arrhythmia. In some embodiments, the disease or disorder is a nervous system disease or disorder. In some embodiments, the disease or disorder is a neuronal disease or disorder. In some embodiments, the nervous system is the central nervous system (CNS). In some embodiments, the nervous system is the peripheral nervous system (PNS). In some embodiments, the nervous system is the CNS, PNS or both. In some embodiments, the CNS disease or disorder is selected from seizures and epilepsy. In some embodiments, the seizures are selected from central and peripheral seizures. In some embodiments, the PNS disease or disorder is selected from seizures, motor neuron spasms, muscle weakness and muscle atrophy. In some embodiments, the disease or disorder is a drug induces disease or disorder. In some embodiments, the disease or disorder is a drug induces symptom or side effect. In some embodiments, the drug is an anti-cancer drug.

In some embodiments, the method comprises administering to the subject a therapeutically effective amount of an agent capable of increasing Ca²⁺ signaling. In some embodiments, the method comprises administering to the subject a therapeutically effective amount of a mitochondrial calcium channel activator. In some embodiments, the method comprises administering to the subject a therapeutically effective amount of an MCU activator. As used herein, the term “MCU activator” encompasses any compound capable of directly or indirectly modulate MCU so as to control or modify mitochondrial Ca²⁺ uptake. In some embodiments, the MCU activator increases the concentration or the effective concentration of Ca²⁺ in the matrix of the mitochondria. In some embodiments, the MCU activator modifies the frequency or rate of Ca2+ oscillation between the mitochondrial matrix the mitochondrial inter membrane space. In some embodiments, the agent that is capable of increasing Ca²⁺ signaling is metformin. In some embodiments, the MCU activator is or comprises metformin.

In some embodiments, the cell proliferation related disease comprises cancer. In some embodiments, the cell proliferation related disease is cancer. As used herein, the term “cancer” refers to any disease characterized by abnormal cell growth. In some embodiments, cancer is further characterized by the potential or ability to invade to other parts of the body beyond the part where the abnormal cell growth originated. In some embodiments, cancer is selected from breast cancer, cervical cancer, endocervical cancer, colon cancer, lymphoma, esophageal cancer, brain cancer, head and neck cancer, renal cancer, meningeal cancer, glioma, glioblastoma, Langerhans cell cancer, lung cancer, mesothelioma, ovarian cancer, pancreatic cancer, neuroendocrine cancer, prostate cancer, skin cancer, stomach cancer, tenosynovial cancer, tongue cancer, thyroid cancer, uterine cancer, and testicular cancer. In some embodiments, the cancer is lung cancer. In some embodiments, the cancer is a solid cancer. In some embodiments, the cancer is a blood cancer. In some embodiments, the cancer is a tumor.

In some embodiments, the method comprises administering an anticancer agent. In some embodiments, the anticancer agent is a chemotherapeutic. In some embodiments, the anticancer agent is a calcium signaling targeting agent. In some embodiments, the anticancer agent is a calcium channel blocker. In some embodiments, the anticancer agent induces CTIA. In some embodiments, the anticancer agent causes CTIA. In some embodiments, the anticancer agent is a first agent.

In some embodiments, the subject is a subject in need of a method of the invention. In some embodiments, the subject is a mammal. In some embodiments, the mammal is a human. In some embodiments, the subject suffers from a proliferation relate disease. In some embodiments, the subject suffers from arrhythmia. In some embodiments, the subject is at risk for developing cancer. In some embodiments, the subject is suspected of having cancer. In some embodiments, the subject is genetically predisposed to cancer. In some embodiments, the subject has cancer. In some embodiments, the subject is undergoing cancer treatment. In some embodiments, the cancer treatment comprises chemotherapy. In some embodiments, the subject suffers from CTIA.

In some embodiments, the subject does not suffer from a metabolic disorder, or any disease or condition associated therewith. In some embodiments, metabolic disorder or any disease or condition associated therewith is diabetes, hyperglycemia, or both. In some embodiments, the subject is not afflicted with diabetes, or any disease associated with the metabolic syndrome. In some embodiments, the subject is not treated with metformin so as to treat the metabolic syndrome, or any disease or condition associated therewith, such as, but not limited to diabetes. In some embodiments, the subject does not suffer from a disease or condition treatable with metformin. In some embodiments, the subject does not suffer from a disease or condition treatable by increasing mitochondrial calcium concentration. In some embodiments, the subject does not suffer from a disease or condition treatable by increasing MCU activity. In some embodiments, the subject does not suffer from a disease or condition treatable with an MCU activator.

In some embodiments, the method further comprises selecting a subject suitable for treatment. In some embodiments, treatment is treatment by a method of the invention. In some embodiments, the method further comprises selecting a subject suitable for treatment with metformin In some embodiments, the method further comprises selecting a subject suitable for treatment with an agent selected from metformin, kaempferol, spermine, A-769662, AICAR, IND 1316, PF 06409577, ZLN 024, Erastin, Honokiol, Ezetimibe, Disulfiram, Efsevin and spermidine. In some embodiments, the selecting comprises excluding a subject afflicted with or at increased risk of developing a metabolic syndrome, or any disease or condition associated therewith, such as diabetes.

By another aspect, there is provided a method of selecting a subject suitable for treatment with an agent capable of:
a. modulating mitochondrial calcium concentration in a tissue of the subject;
b. modulating mitochondrial calcium channel activity in the tissue; or
c. a combination there thereof;
   the method comprising determining the presence of electro-mitochondrial desynchronization in the subject, wherein the presence of said desynchronization indicates the subject is suitable for treatment.
   In some embodiments, the subject suffers from a disease or disorder. In some embodiments, the disease or disorder is one that is known to potentially comprise or can be characterized by electro-mitochondrial desynchronization. In some embodiments, in some embodiments, the tissue is a diseased tissue. In some embodiments, the tissue is a tissue of the disease or disorder. In some embodiments, the tissue is a tissue of the disease or disorder in the subject.

### Array and System

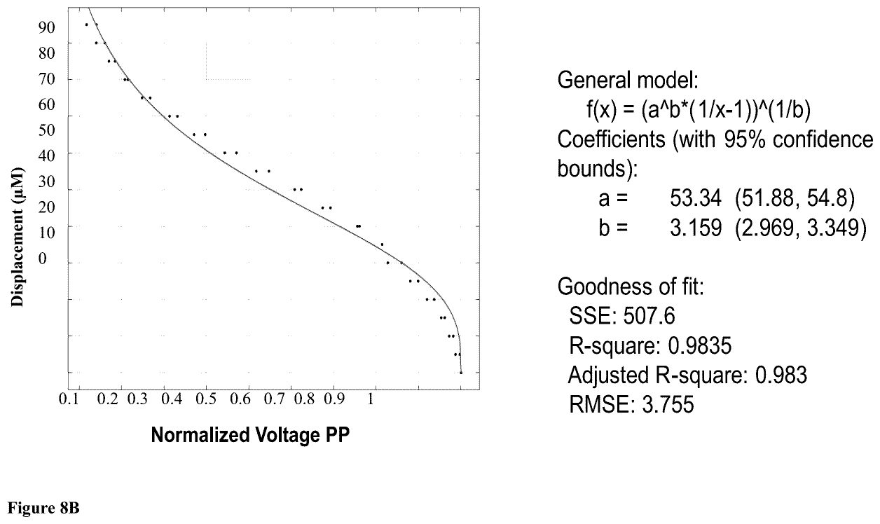

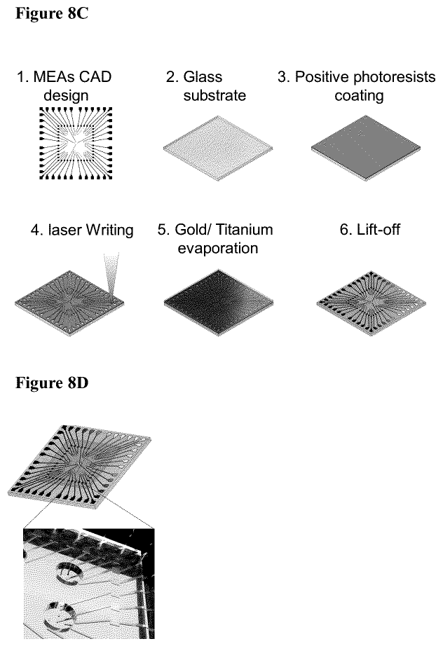

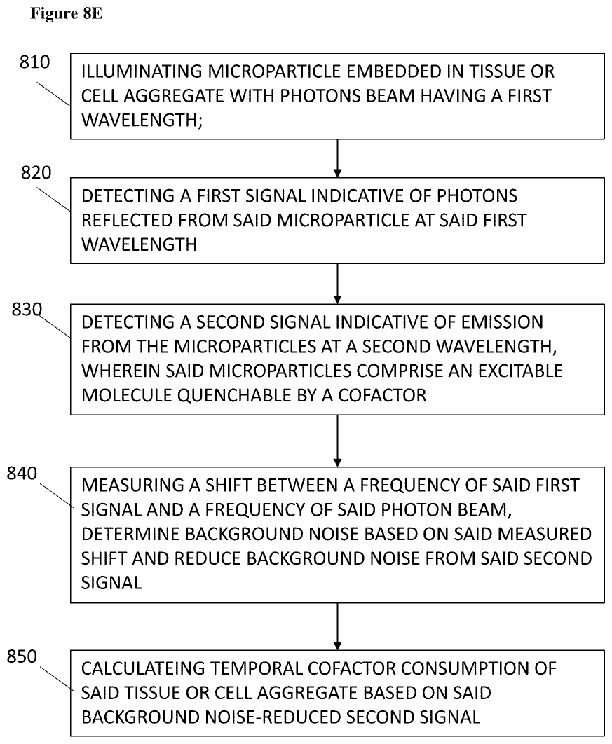

Reference is now made to FIG. 8A which is an illustration of a prior art sensing system and a system according to some embodiments of the invention. Prior art system 10 may include an illumination source 110 configured to emit light at a first wavelength, for example, a LED laser emitting light at 532 nm. System 10 may further include a PMT sensor 140 configured to detect a signal indicative of emission from microparticles embedded in a tissue or cell aggregate 5 at a second wavelength, for example, 605 nm. In some embodiments, the microparticles comprise an excitable molecule quenchable by a cofactor. In some embodiments, a controller, not illustrated, is configured to calculate temporal cofactor consumption of the tissue or cell aggregate based on the first and second signals, for example, the oxygen levels in tissue or cell aggregate 5. In some embodiments, the cofactor is oxygen.

A system 100, according to some embodiments of the invention may include, illumination source 110 configured to emit light (a photon beam 115) at a first wavelength, for example, a LED laser emitting light at 532 nm, a first PMT sensor 120 and a controller 130. In some embodiments, first PMT sensor 120 may be configured to detect photons at the first wavelengths reflected from tissue or cell aggregate 5. In some embodiments, system 100 may further include second PMT sensor 140 configured to detect a signal indicative of emission from microparticles embedded in a tissue or cell aggregate 5 at a second wavelength. In some embodiments, system 100 may include an optical element 125 configured to direct the reflected photons to first PMT sensor 120 and the emitted photons to second PMT sensor 140.

Controller 130 may be configured to control illumination source 110 to emit light at the first wavelength and to receive signals from first PMT sensor 120 and second PMT sensor 140. Controller 130 may be any suitable computing device capable of controlling illumination source 110 and receiving signals from PMT sensors 120 and 140. Controller 130 may include a processing unit (e.g., CPU), a memory, and any input/output device(s). Controller 130 may be configured to execute methods according to some embodiments of the invention, for example, the method of FIG. 8E.

Reference is made to FIG. 8E which is a flowchart of a method of measuring properties of a tissue or cell aggregates according to some embodiments of the invention. In step 810, microparticles embedded in tissue or cell aggregate 5 may be illuminated with photons beam having a first wavelength. For example, controller 130 may control illumination source 110 to emit photons beam having a wavelength of 532 nm (e.g., the LED laser illustrated in FIG. 8A).

In step 820, a first signal indicative of photons reflected from the microparticles may be detected by a first sensor (e.g., first PMT sensor 120) at the first wavelength. For example, controller 130 may receive from first PMT sensor 120 a first signal 155 indicative of emission from microparticles embedded in tissue or cell aggregate 5 at a wavelength of 605 nm as illustrated in FIG. 8A.

In step 830, a second signal indicative of emission from the microparticles, may be detected by a second sensor (e.g., second cPMT sensor 140) at a second wavelength, wherein said microparticles comprise an excitable molecule quenchable by a cofactor. For example, photons having a wavelength of 605 nm may be reflected from the microparticles in tissue or cell aggregate 5 and detected by second PMT sensor 140 and controller 130.

In step 840, a shift between a frequency of said first signal and a frequency of said photon beam, is measured and background noise is determined based on said measured shift. The background noise is reduced from said second signal. For example, second signal 160 may be used to reduce the noise of signal to produce the clean smooth signal 150. In some embodiments, the raw signal may be signal 155 and signal 150 is the filtered signal. In some embodiments, the filtering may include measuring a shift between a frequency of second signal 160 and a frequency of first signal 155; determining background noise based on the measure shift; and reducing the background noise form the second signal to receive filtered signal 160.

In step 850, a temporal cofactor consumption of said tissue or cell aggregate may be calculated based on said background noise-reduced second signal. In some embodiments, the temporal cofactor consumption may be the oxygen level in tissue or cell aggregate 5.

In some embodiments, a temporal change in intensity of said first signal may be detected. In some embodiments, a relative displacement of said microparticle may be calculated, based on said detected change. In some embodiments, the detected change in the intensity of the signal is proportional to the relative displacement of the microparticles, for example, the distance of the focal point may be measured by measuring the movement of an x-y table (e.g., a mechanical stage), holding system 100, (e.g., in micrometers) when the focal point of the microscope lens moved away from the microparticles in tissue or cell aggregate 5 due to the displacement. In order to re-focus the lens on the microparticles the x-y table is moved, and the length of the movement is measured. For example, the distance between two consecutive maxima, in signal 160, are proportional to the relative displacement. In some embodiments, displacement is measured in an axis perpendicular to photons beam 115. In some embodiments, the displacement is contraction of the organoid. In some embodiments, the displacement is organoid beating. It will be understood by a skilled artisan that any measure of the contraction can be measured by the displacement including but not limited to, the frequency of contraction and the magnitude of contraction.

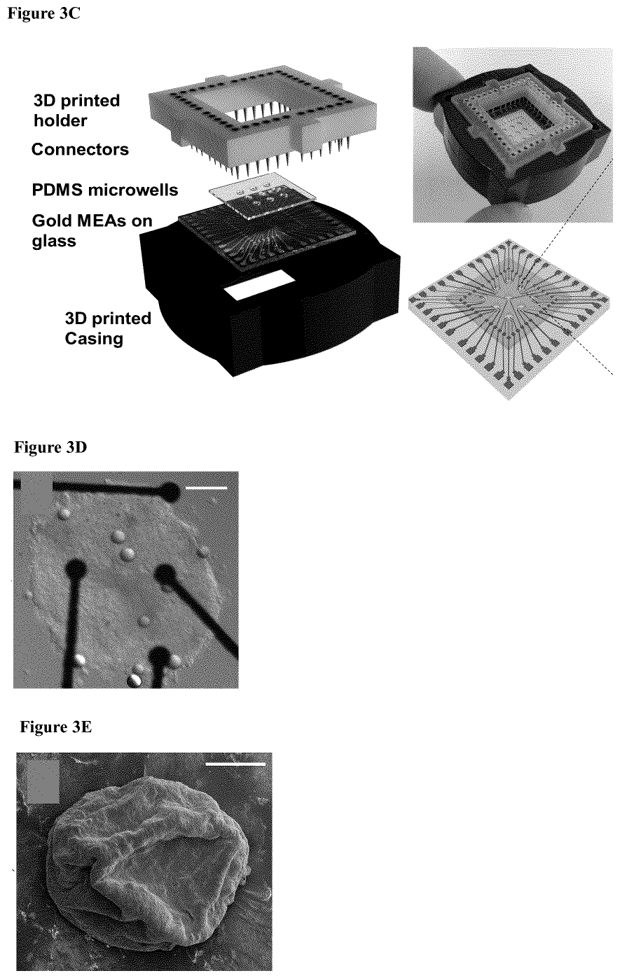

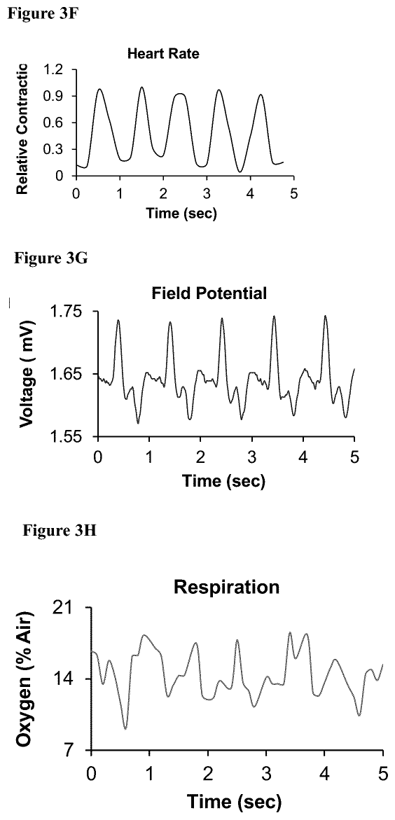

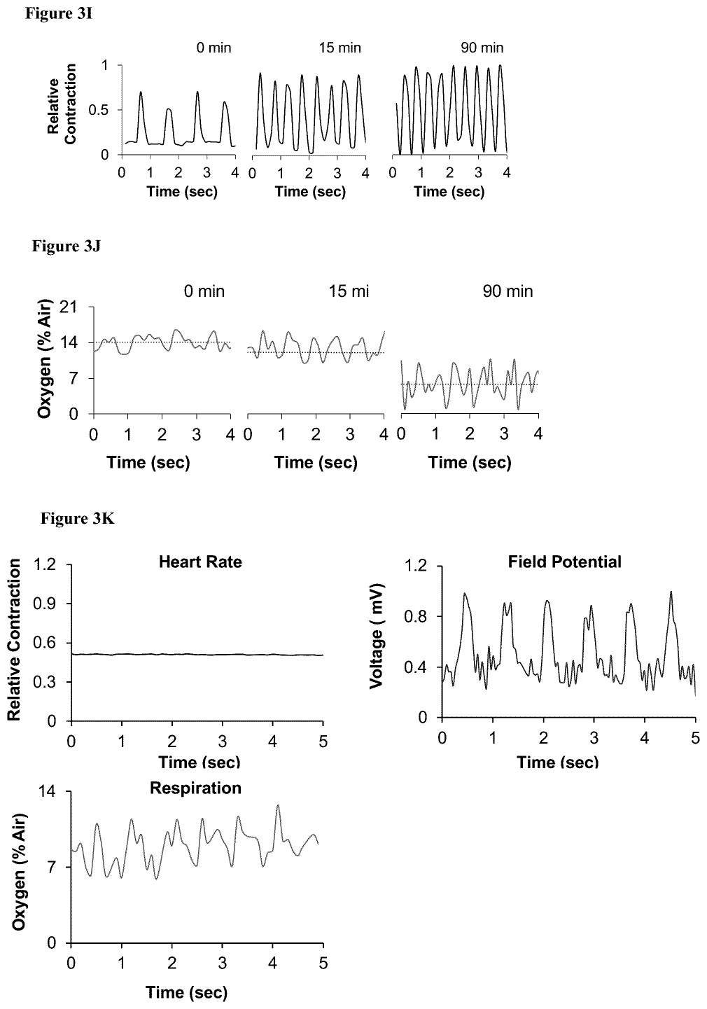

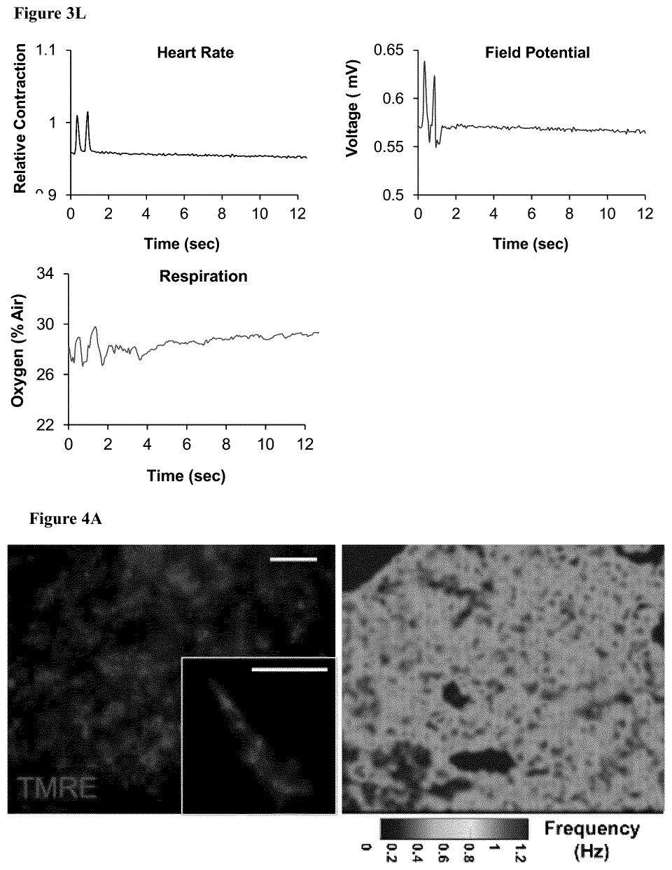

In some embodiments, the method may further include sensing field potential (as illustrated in the example, of FIG. 3G) of the tissue or cell aggregates from an array of microelectrodes (not illustrated) for measuring the electrical activity of the tissue or cell aggregates simultaneously to detecting the first signal. In some embodiments, the method may further include comparing frequencies of the first signal, the second signal, and the field potential of the tissue. A nonlimiting example, for such comparison when the tissue is simulating a beating heart, is illustrated in FIG. 3F (when the displacement is indicative of heartbeat), 3G (when the electrical activity is indicative of the electrical activity of the heat), and 3H (when the oxygen level is indicative of respiration). In some embodiments, if the comparison between the frequencies yields a deviation lower than a threshold, the comparison is indicative of a healthy tissue or cell aggregates, for example, a healthy heart.

By another aspect, there is provided a system comprising an illumination source, a first photomultiplier tube (PMT) sensor; and a controller.

In some embodiments, the control is configured to control the illumination source to illuminate a microparticle embedded in a tissue or cell aggregate with a photon beam. In some embodiments, the photon beam has a first wavelength. In some embodiments, the control is configured to control the illumination source to illuminate a tissue or cell aggregate with a photon beam. In some embodiments, the control is configured to detect a first signal indicative of photons reflected from the microparticles. In some embodiments, the detecting a first signal is by the first PMT sensor. In some embodiments, the first signal is at a first wavelength. In some embodiments, the photons reflected from the microparticles are at the first wavelength. In some embodiments, the control is configured to detect a change in intensity of the first signal. In some embodiments, the control is configured to calculate displacement of the microparticles. In some embodiments, displacement is relative displacement. In some embodiments, the calculation is based on the detected change. In some embodiments, the detected changes in the intensity of the first signal is indicative of the relative displacement of the microparticle. In some embodiments, the displacement is measured in an axis perpendicular to the photons beam. In some embodiments, the displacement is a measure of movement of the tissue or cell aggregate. In some embodiments, movement is contraction.

In some embodiments, the system is a sensing system. In some embodiments, the sensing is sensing a parameter in the tissue or cell aggregate. In some embodiments, the system comprises a receptacle for the tissue or cell aggregate. In some embodiments, the cell aggregate is an organoid. In some embodiments, the organoid is a cardiac organoid. In some embodiments, the organoid is a brain organoid. In some embodiments, the organoid is a cardiac organoid of the invention. Methods of making brain organoids are well known in the art and any such method may be employed to produce an organoid to be sensed.

In some embodiments, the system further comprises a second PMT sensor. In some embodiments, the control is configured to detect a second signal. In some embodiments, the second signal is detected by the second PMT sensor. In some embodiments, the second signal is indicative of emission from microparticles embedded in the tissue or cell aggregate. In some embodiments, the second signal is at a second wavelength. In some embodiments, the second wavelength is a different wavelength than the first wavelength. In some embodiments, the first and second signal are sufficiently different to not overlap. In some embodiments, the first and second signal are at different band passes. In some embodiments, the first signal comprises a wavelength of about 532 nm. In some embodiments, the photon beam comprises a wavelength of about 532 nm. In some embodiments, the photon beam and first signal comprise the same wavelength. In some embodiments, the second signal comprises a wavelength of about 605 nm. In some embodiments, the microparticles comprise an excitable molecule or moiety quenchable by a cofactor. In some embodiments, the control is configured to calculate temporal cofactor consumption in the tissue of cell aggregate. In some embodiments, the calculating is based on the second signal. In some embodiments, the calculating is based on the second signal and the first signal.

In some embodiments, the cofactor is a biological compound. In some embodiments, the compound is produced in the tissue or cell aggregate. In some embodiments, the cofactor is oxygen (O2). In some embodiments, the excitable molecule or moiety is excitable by the photon beam. In some embodiments, the excitable molecule or moiety upon excitation emits at the second wavelength. In some embodiments, an excitable molecule or moiety is phosphorescent. In some embodiments, the microparticles are oxygen sensors. Oxygen sensors that are quenchable and emit a detectable signal are well known in the art and any such sensor maybe used. In some embodiments, the quenchable sensor comprises an oxygen quenchable luminescent dye. Such dyes are well known in the art. In some embodiments, the quenchable sensor comprises ruthenium. Examples of such can be found in McEvoy et al., 1996, “Dissolved oxygen sensor based on fluorescence quenching of oxygen-sensitive ruthenium complexes immobilized in sol-gel-derived porous silica coatings”, doi.org/10.1039/AN9962100785, Wang et al., 2014, “Optical methods for sensing and imaging oxygen: materials, spectroscopies and applications”, hem. Soc. Rev., 2014, 43, 3666-3761, and Achatz et al., 2010, “Luminescent Sensing of Oxygen Using a Quenchable Probe and Upconverting Nanoparticles”, Angew Chem Int Ed Engl. 2011 Jan. 3; 50(1):260-3, herein incorporated by reference in their entirety. In some embodiments, the temporal cofactor consumption is the cofactor level in the tissue or cell aggregate. In some embodiments, the temporal cofactor consumption is proportional to the cofactor level in the tissue or cell aggregate.

In some embodiments, the controller is configured to filter the second signal. In some embodiments, the filtering is with a parameter of the photon beam. In some embodiments, the filtering is with the first signal. In some embodiments, the filtering is with a difference between the photon beam and the first signal. In some embodiments, the difference is the shift between the photon beam and the first signal. In some embodiments, shift is shift in frequency. In some embodiments, shift is shift in wavelength. In some embodiments, the filtering comprises measuring a shift between a frequency of the first signal and a frequency of the photon beam. In some embodiments, the filtering comprises measuring a shift between a wavelength of the first signal and a wavelength of the photon beam. In some embodiments, the filtering comprises determining background based on the measured shift. In some embodiments, background is background noise. In some embodiments, filtering comprises reducing the background from the second signal. In some embodiments, filtering comprises filtering the background from the second signal. In some embodiments, filtering comprises cleaning the second signal.

In some embodiments, the controller is further configured to sense field potential of the tissue or cell aggregate. In some embodiments, the field potential is from an array of microelectrodes. In some embodiments, the array is in the tissue or cell aggregate. In some embodiments, the array is in the receptacle holding the tissue or cell aggregate. In some embodiments, the microelectrodes are for measuring the electrical activity in the tissue or cell aggregate. In some embodiments, in the tissue or cell aggregate is of the tissue or cell aggregate. In some embodiments, the sensing field potential is simultaneous to the detecting the first signal. In some embodiments, the sensing field potential is simultaneous to the detecting the second signal. In some embodiments, the sensing field potential is simultaneous to the detecting the first signal and the second signal. In some embodiments, the control is configured to compare frequencies of the first signal and the second signal. In some embodiments, the control is configured to compare frequencies of the first signal and the field potential. In some embodiments, the control is configured to compare frequencies of the second signal and field potential. In some embodiments, if the comparison between the frequencies yields a deviation lower than a predetermined threshold, the low deviation is indicative of a healthy tissue or cell aggregate. In some embodiments, if the comparison between the frequencies yields a deviation greater than a predetermined threshold, the high deviation is indicative of a tissue or cell aggregate with a disease or disorder. In some embodiments, the disease or disorder comprises electro-mitochondrial desynchronization. In some embodiments, the high deviation is indicative of electro-mitochondrial desynchronization.

By another aspect, there is provided a use of the system of the invention in making measurements in a tissue or cellular aggregate.

By another aspect, there is provided a method of evaluating cellular function, the method comprising:
a. placing a tissue, an organoid or a cellular aggregate in a system of the invention;
b. applying a condition to the tissue, organoid or cellular aggregate; and
c. measuring at least cofactor consumption in the tissue, organoid or cellular aggregate;
thereby evaluating cellular function.

In some embodiments, the method comprises placing the tissue or cellular aggregate in the system. In some embodiments, the tissue or cellular aggregate is an organoid. In some embodiments, the organoid is a cardiac organoid. In some embodiments, the cardiac organoid is capable of beating. In some embodiments, the cardiac organoid is multichambered. In some embodiments, the cardiac organoid is a cardiac organoid of the invention.

In some embodiments, the measuring comprises measuring cofactor consumption, displacement and electrical field potential. In some embodiments, a significant deviation in at least one of displacement, cofactor consumption, and electrical field potential after applying the condition is indicative of electro-mitochondrial desynchronization. In some embodiments, a significant deviation in at least two of displacement, cofactor consumption, and electrical field potential after applying the condition is indicative of electro-mitochondrial desynchronization. In some embodiments, a significant deviation in all of displacement, cofactor consumption, and electrical field potential after applying the condition is indicative of electro-mitochondrial desynchronization. In some embodiments, deviation is deviation from a control. In some embodiments, the control is displacement, cofactor consumption, and/or electrical field potential before applying the condition. In some embodiments, the control is displacement, cofactor consumption, and/or electrical field in a control tissue, organoid or cellular aggregate. In some embodiments, the control is a healthy control. In some embodiments, a control is an untreated control. In some embodiments, the control is a control that does not have electro-mitochondrial desynchronization.

By another aspect, there is provided a method of testing a therapeutic agent for cardiac side effects, the method comprising placing a cardiac organoid with the system and adding the therapeutic agent, thereby testing a therapeutic agent for cardiac side effects.

By another aspect, there is provided a method of testing a cardiovascular agent, the method comprising placing a cardiac organoid with the system and adding the cardiovascular agent, thereby testing a cardiovascular agent.

In some embodiments, the agent is not a cardiovascular drug. In some embodiments, the agent is a cardiovascular drug. In some embodiments, a cardiovascular drug is a cardiac drug. In some embodiments, the method further comprises measuring at least one of displacement, cofactor consumption, and electrical field potential in said cardiac organoid. In some embodiments, at least two of displacement, cofactor consumption, and electrical field potential are measured. In some embodiments, all of displacement, cofactor consumption, and electrical field potential are measured. In some embodiments, displacement is contraction of the cardiac organoid. In some embodiments, cofactor consumption is metabolism in the cardiac organoid. In some embodiments, the cofactor is oxygen. In some embodiments, metabolism is respiration. In some embodiments, metabolism comprises mitochondrial function. In some embodiments, field potential is field potential duration. In some embodiments, field potential is field potential amplitude. In some embodiments, field potential is spontaneous field potential. In some embodiments, field potential comprises electrical activity in the organoid.

In some embodiments, a deviation in any one of displacement, cofactor consumption, and electrical field potential indicates a cardiac side effect. In some embodiments, a deviation is an imbalance between displacement, cofactor consumption, and electrical field potential. In some embodiments, a deviation is a change that is not compensated by the other conditions. It will be understood that a therapeutic agent could increase displacement, consumption and potentials all in equal proportion. This would not constitute a deviate as all the measures are still in proportion but have merely increased. A deviation is thus an imbalance between the three measures. In some embodiments, a deviation in any one of displacement, cofactor consumption, and electrical field potential indicates an effect of the agent. In some embodiments, a deviation is a significant deviation. In some embodiments, significant is statistically significant. In some embodiments, significant is a deviation greater than a predetermined threshold. In some embodiments, the deviation is after administering the drug as compared to before the drug was administered. In some embodiments, the deviation is after administering the drug as compared to a control in which the drug was not administered. In some embodiments, the control is an untreated control. In some embodiments, the deviation is in any two of displacement, cofactor consumption, and electrical field potential. In some embodiments, the deviation is in all of displacement, cofactor consumption, and electrical field potential. It will be understood that a change in any of displacement, cofactor consumption, and electrical field potential indicates that the drug has had an effect on the cardiac organoid and would therefore have an effect on a heart of a subject as well. In some embodiments, a deviation is an imbalance between displacement, cofactor consumption, and electrical field potential. In some embodiments, the imbalance is between contraction and mitochondrial function. In some embodiments, the imbalance is indicative of arrhythmia. In some embodiments, the imbalance is indicative of a therapeutic effect on contraction rhythm.

### General

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Cell Culture

Human induced pluripotent stem cell lines: UN-1, ACS-1021, and ACS-1028 were cultured on growth factor reduced Matrigel (BD Biosciences, San Jose, CA) in mTeSR-1 media (StemCell Technologies, Canada) with daily medium changes. Cells were obtained and tested for mycoplasma contamination using PCR. Cells were serially passaged using 0.5× TrypLE Enzyme (Thermo Fisher Scientific, Waltham, MA, USA) supplemented with 0.5 mM EDTA (Invitrogen, Thermo Fisher Scientific).

HEK 293T cells (ATCC, USA) were seeded in 10 cm cell culture plates at a density of 4×10\hat{ }6 cells/plate. The cells were maintained in 293T medium composed of DMEM high glucose (4.5 g/l; Merck, USA) supplemented with 10% FBS (BI, Israel), 1× NEAA (BI, Israel), and 2 mM L-alanine-L-glutamine (BI, Israel).

Rat primary cardiac microvascular endothelial cells (CECs; Vec Technologies, USA) were cultured on gelatin-coated flasks using Endothelial Cell Growth Medium-2MV (Lonza, Switzerland) according to the manufacturer's directions.

All cells were cultured under standard conditions in a humidified incubator at 37° C. under 5% CO2.

### Finite Element Analysis

Finite element models were created using the Multiphysics static structural model of SolidWorks® 2018. Numerical simulation and investigation were done in several conditions of free-cardiac mass on low-adherent plate surface, a homogenous-geometrically confined cardiac mass embedded in soft matrix and heterogeneous-geometrically confined cardiac mass embedded in the soft matrix and patterned by rigid microvascular structures. Initial mass was simulated as a 500 μm diameter sphere with tissue parameters defined by Poisson's ratio of 0.4, the density of 1.06 g/ml, and isotropic spring constant of 1 Pa/m². Physiological Young's modulus of 4 kPa was chosen between 1.25 kPa measured for cardiomyocytes and 10-20 kPa of adult tissue. An isotropic spring foundation of 1 Pa/m² was applied to its lower surface, simulating a glass plate bottom.

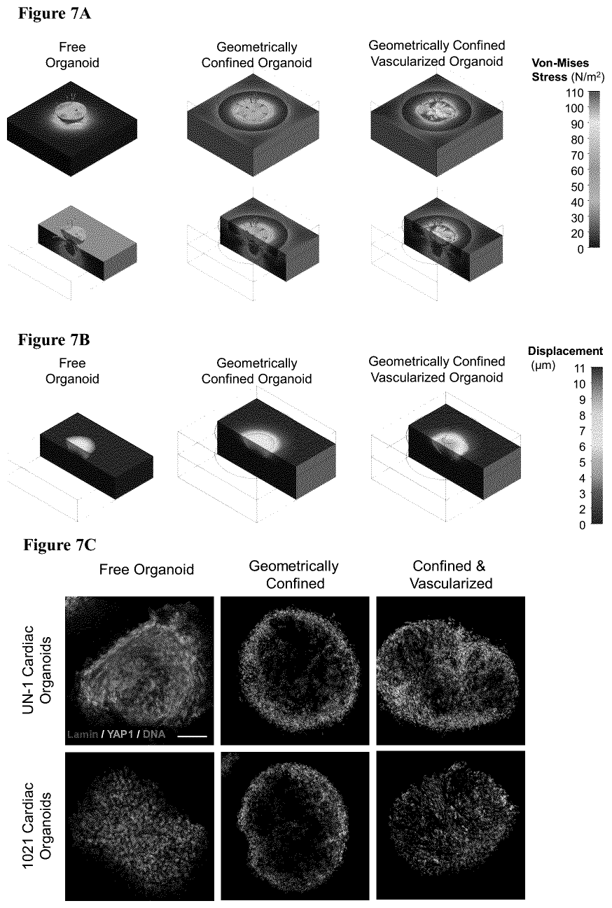

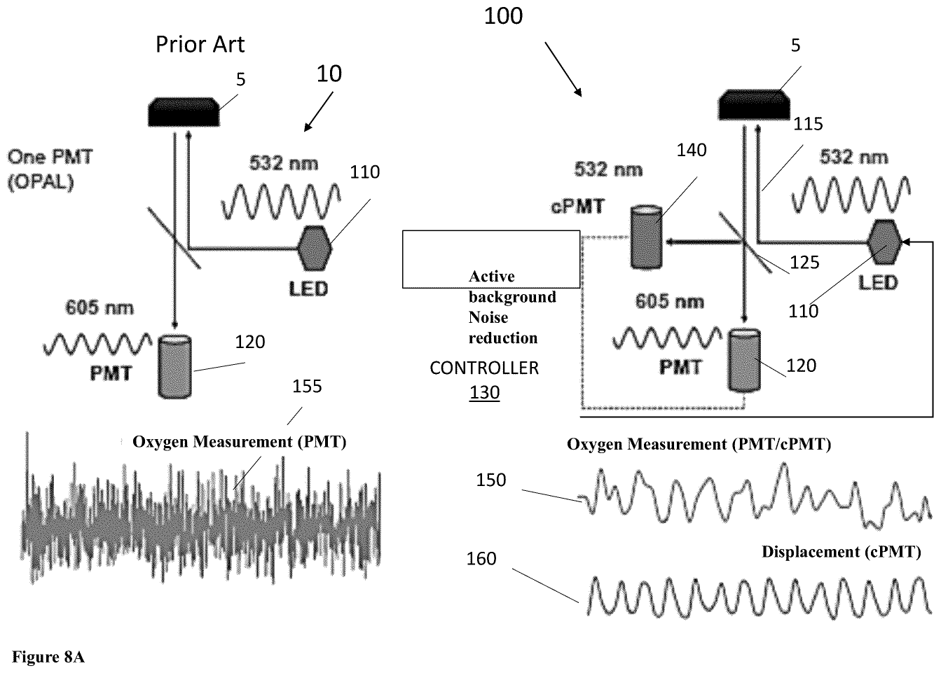

Confinement was simulated using a 1.5 mm diameter silicone well (defined by Young's modulus of 800 kPa, Poisson's ratio of 0.45, density of 0.965 g/ml, and isotropic spring constant of 1 Pa/m²) and surrounding matrix defined by Young's modulus of 1 kPa, Poisson's ratio of 0.5, density of 1.03 g/ml, and isotropic spring constant of 1 Pa/m². Microvascular structures were simulated by random patterns on 30 μm diameter microvascular-like structures defined by Poisson's ratio of 0.4, density of 1.03 g/ml, and isotropic spring constant of 1 Pa/m². Physiological Young's modulus of 50 kPa was chosen between 10 kPa estimated for capillaries and 300 kPa of arteries. The static load model was applied using isotropic contraction of 350 μN causing Gaussian displacement fitted to the measured curvature resulting in 5-15 μm of radial displacement (FIG. 7B). The mesh used minimum and maximum element sizes of 150 and 165 m, respectively.

Cardiac Differentiation of Human iPSC

The human induced pluripotent stem cells (hiPSCs) were seeded on Matrigel in mTeSR-1 medium and allowed to reach 80-90% confluence. Cardiac differentiation was conducted as previously described. Briefly, a basal medium named CDM3 was used (RPMI-1640, 500 μg/mL recombinant human serum albumin, 213 μg/mL L-ascorbic acid 2-phosphate and 1% penicillin/streptomycin). At day zero of differentiation, the medium was replaced with CDM3 supplemented with 6 μmol/L CHIR99021 (Stemgent) for two days. On day two of the differentiation, the culture medium was switched to CDM3 medium supplemented with 2 μM Wnt-C59 (Selleckchem), for an additional two days. From day four onward, cells were cultured with RPMI supplemented with B27 without Insulin; (Gibco, USA).

### Generation of GFP-Reported CECs

Plasmid encoding GFP reporter upstream of a minimal CMV promoter were purchased from System Biosciences and validated in-house. Plasmids were acquired as bacterial LB-agar stabs and used per the provider's instructions. Briefly, each stab was first seeded into agar LB (Bacto Agar; BD, USA) in 10 cm plates. Then, single colonies were inoculated into flasks containing LB (BD Difco LB Broth, Lennox; BD, USA) and 100 μg/ml penicillin (BI, Israel). Transfection grade plasmid DNA was isolated from each flask using the ZymoPURE II Plasmid Maxiprep Kit (Zymo Research, USA) according to the manufacturer's instructions.

HEK 293T cells were transfected with a GFP-expressing plasmid and the packaging plasmids using the TransIT-LT1 transfection reagent (Mirus Bio, USA) according to the provider's instructions. Briefly, 6.65 μg GFP lentivector plasmid, 3.3 μg pVSV-G, and 5 μg psPAX2 were mixed in Opti-MEM reduced serum medium (Gibco, USA), with 45 μl of TransIT-LT1, kept at room temperature to complex and then added to each plate. Following 18 h of incubation, the transfection medium was replaced with 293T medium and virus-rich supernatant was harvested after 48 h and 96 h. The supernatant was clarified by centrifugation (500×g, 5 min) and filtred (0.45 μm, Millex-HV, MerckMillipore). Packaging plasmids were a kind gift of the Nissim Benvenisti Lab, HUJI, Jerusalem, Israel.

### Organoid Culture

hiPS-derived cardiomyocytes and microvascular cardiac endothelial cells (VEC Technologies, USA) were counted and mixed in a 2:1 ratio in growth factor reduced Matrigel (BD Biosciences, San Jose, CA) at a cell density of 6.8×10⁴ cells per μl. A volume of 1.1 μl of the gel-imbedded mixture (7.48×10\hat{ }4 cells) was injected into each microwell and left to form spontaneously in RPMI-1640 supplemented with B27 minus insulin and 5 ng/ml vascular endothelial growth factor (VEGF-A, Peprotech) until their spontaneous beating was regained after 6-10 days.

### Immunostaining of Cardiac Organoids

Cardiac organoids were fixed with 4% paraformaldehyde (PFA) for 1 hour on ice and washed 3 times with Dulbecco's Phosphate Buffered Saline with calcium and magnesium (DPBS), (Sigma-Aldrich, USA). Samples were incubated for 1 hour at room temperature with 100 mM glycine and washed for 30 minutes with DPBS. Permeabilization was carried out overnight at 4° C. with 0.5% Triton X-100 in DPBS. Blocking buffer was composed of 3.8 g NaCl, 0.94 g NaHPO₄, 0.2 g NaH₂PO₄, 5 g bovine serum albumin Fraction V (MP Biomedicals, USA), 0.5% Triton X-100, and 0.25 ml Tween-20 in 50 ml distilled water. Samples were incubated in blocking buffer for 48 hours at 4° C., washed and incubated with primary antibodies diluted in blocking buffer for an additional 48 hours at 4° C. Samples were washed 24 hours at room temperature before the addition of secondary antibodies diluted in blocking buffer, and 48 hours incubation at 4° C. Nuclei were counterstained with Hoechst 33258 (Sigma-Aldrich, USA) at 1:1,000 concentration. The sample was washed for 24 h before microscopy. Confocal microscopy was performed on an LSM-700 Zeiss microscope.

### Antibodies

Rabbit Anti-cTNT Abcam: ab45932 Concentration 1:100; Mouse Anti-α-Actinin thermofisher: A7811 Concentration 1:100; Rabbit Anti-MLC2v Abcam: ab79935 Concentration 1:100; Rabbit Anti-WT1 Abcam: ab89901 Concentration 1:100; Rat Anti-HCN4 Abcam: ab32675 Concentration 1:100; Mouse Anti-SHOX2 Abcam: ab55740 Concentration 1:100; Rabbit Anti-Cardiac Troponin I Abcam: ab52862 Concentration 1:100; Rabbit Anti-Periostin Abcam: ab215199 Concentration 1:100; Mouse Anti-CD31 Abcam: ab215199 Concentration 1:100; Rabbit Anti-TBX18 Abcam: ab115262 Concentration 1:100; Donkey Anti-Rabbit Alexa Fluor 594 Jackson ImmunoResearch: 711-585-152 Concentration 1:100; Donkey Anti-Mouse Alexa Fluor 488 Jackson ImmunoResearch: 715-546-150 Concentration 1:100; Donkey Anti-Rat Alexa Fluor 647 Jackson ImmunoResearch: 712-605-150 Concentration 1:100; and Donkey Anti-Mouse Alexa Fluor 647 Abcam: ab150111 Concentration 1:100.

### RNA-Seq Analysis

RNA-seq data for adult and fetal human cardiomyocytes was downloaded from GSEA series GSE126573 along with accompanying metadata. RNA extraction from cell cultures was performed using RNeasy Micro kit (Qiagen, USA) according to the manufacturer's direction. Library preparation and RNA sequencing were performed by the Hebrew University Center for Genomic Technologies. Briefly, library construction was conducted using Illumina TruSeq RNA Library Prep V2 Kit (Illumina, USA) and sequenced on Illumina NextSeq500 with single-end, 86 bp reads using the High Output V2 Kit. Sequencing reads were mapped to the UCSC human transcriptome (genome build hg19) using Bowtie2. Expression levels of all genes were quantified using RSEM, yielding an expression matrix of inferred gene counts. Differential expression analysis was performed using R package DESeq2 using the default parameter. Reported are p-values from a negative binomial Wald-test.

### Visual Contraction Analysis

Fast phase-contrast imaging (17 frames per second) was performed at suitable magnification using an LSM-700 Zeiss microscope. The time-lapse micrograph was analyzed using a custom MATLAB® code. Briefly, multiple regions of interest (ROIs) were selected on the cardiac tissue borders, producing a distinct area with changes representative of the cardiac contraction. Dynamic changes in the ROI intensity were measured over time and converted into beating rates using Fast Fourier Transformation (FFT). Contractility was calculated based on the frequency amplitude magnitude.

### Optical Permissive Confined Microelectrode Array (MEA) Fabrication

Fabrication procedures were conducted in a class 100 cleanroom environment at the Hervey M. Krueger Family Center of Nanoscience and Nanotechnology at the Hebrew University of Jerusalem. Microelectrode array and disposable PDMS microwell insert design were carried out using CleWin 5® and SolidWorks® (SolidWorks, USA). A standard commercialized 75×50×1 mm microscope slides (Corning®) were cleaned in turn by isopropanol, piranha solution (a mixture of sulfuric acid, water, and hydrogen peroxide), and deionized water. Electrode patterns were etched in a double positive-tone photoresist (AZ1505 and LOR 5b) using direct laser writing (Microtech@, LE405-A), followed by a 2 minutes development in AZ 726 MIF. The pattern was sputtered with a 10 nm titanium layer, and a 150 nm Gold using a Thin Film Deposition System Vacuum Coater (TFDS-141®; VST). Electrodes were then formed using a 2 minutes lift-off process in 100% AR grade acetone. PDMS microwell inserts were fabricated using laser cutting. Briefly, a thin sheet of PDMS (Dow Corning) was cast to 0.7 mm height using a motorized film applicator (Erichsen) and cured at 70° C. for 1 h. Microwells were cut to 1.5 mm diameter and a center-to-center distance of 3 mm using a 355 nm pulsed Nd-YAG laser (3D-Micromac). PDMS inserts were washed with 70% (vol/vol) Ethanol (EtOH), nitrogen dried, and covalently bound to clean 0.5 mm thick glass coverslips (Schott) or clean optical permissive microelectrode arrays using oxygen plasma activation.

### 3D Printing of Cardiac Microphysiological Platform

Microelectrode connectors and casing design was carried out using Solidworks®. SUP706®, VeroBlackPlus@, and VeroWhitePlus@ were deposited according to a designed pattern using a Connex3 Objet260® 3D printer. The 3D printed parts were cleaned from the support material (SUP706®) overnight in 2% Sodium hydroxide and 4% sodium metasilicate solution. The printed parts were fitted with connectors and assembled into a microrheological platform and assembled with metal connectors. Prior to use, the complete platform was cleaned overnight in 70% Ethanol and sterilized for 3 hours in UV.

### Electrophysiology Recording

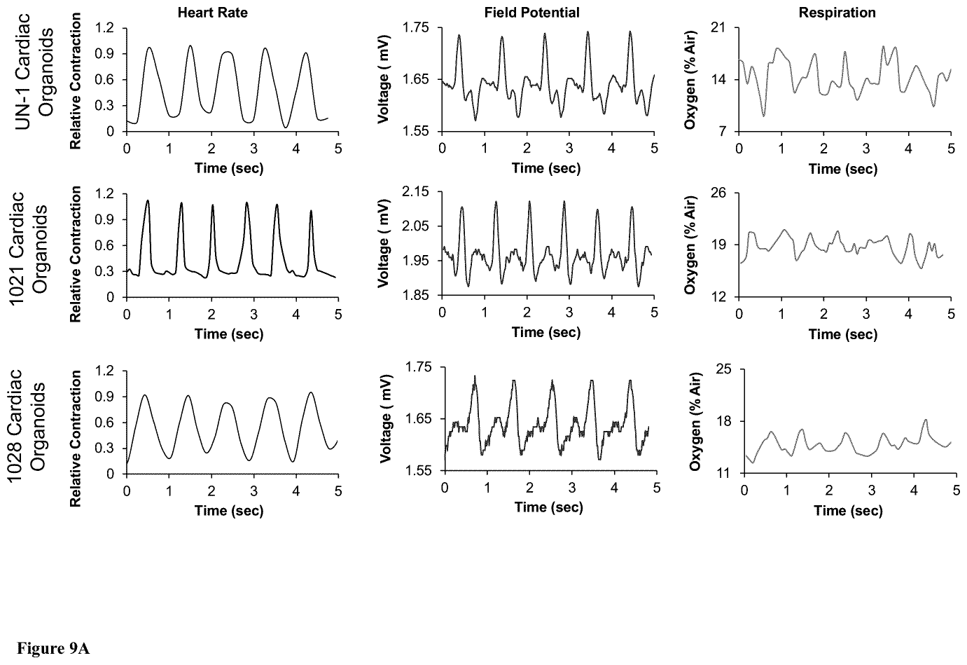

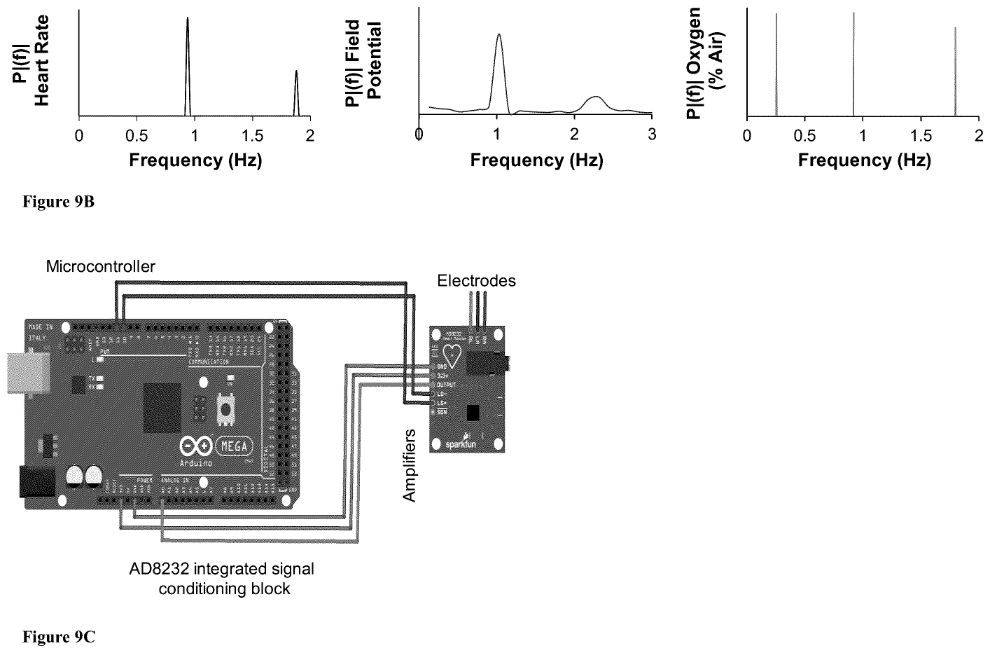

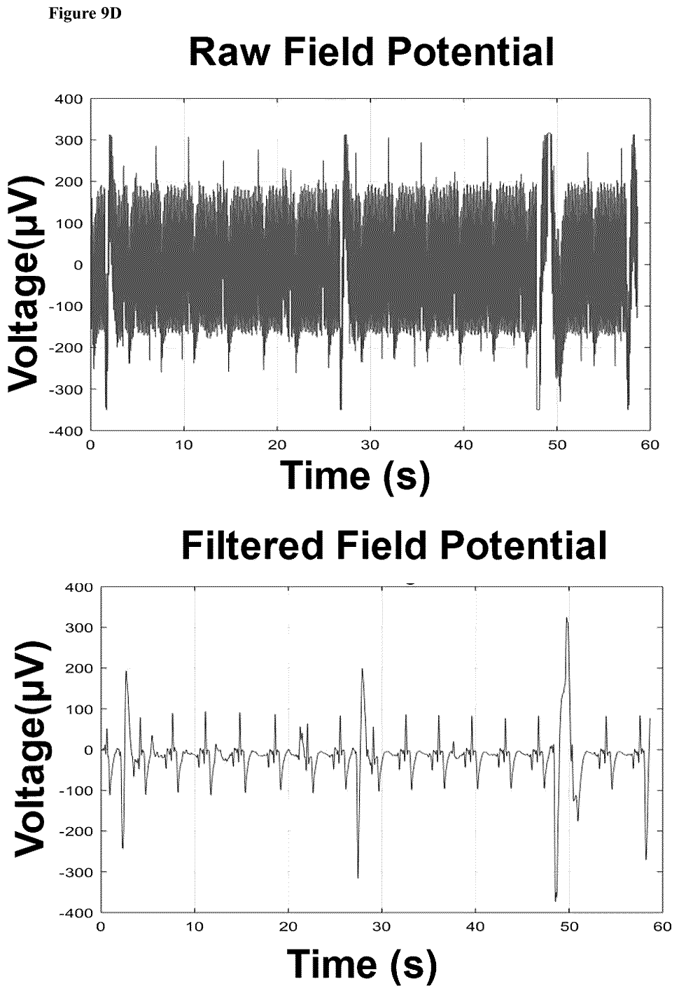

The MEA design and printed connections enabled access to 45 different electrodes inside the platform intermittently via a compatible sensor circuit (FIGS. 9A-9B). Spontaneous cardiac field potentials (FPs) were recorded from cardiac organoids using an integrated signal conditioning circuit (AD8232®), with a 2-pole adjustable high-pass filter, 3-pole adjustable low-pass filter, adjustable gain, and medical instrumentation amplifiers to extract, amplify, and filter the extracellular field potential of the organoids remotely within the confined well. The custom design circuit was connected to Arduino MEGA 2560® microcontroller acting as an analog-to-digital converter (ADC) to the integrated control system. Spontaneous cardiac field potentials were measured at 100 Hz sampling rate in each microwell. Field potential rhythm frequency was calculated in real-time using a custom-made MATLAB® software imposing Fourier Transformation (FFT) on the evolving kinetic data (FIGS. 9C-9D).

### Real-Time Oxygen and Contraction Rate Measurement

Real-time oxygen measurement was performed based on previously described elements. Briefly, phosphorescent ruthenium microprobes (CPOx-50-RuP) show a decrease in phosphorescence decay time as a function of oxygen concentration, allowing to measure the oxygen content. Controlled sinusoidal modulated 532-nm LED signal excites the embedded oxygen sensors and emits a sinusoidal amplitude-modulated light at 605 nm that is shifted in phase due to oxygen quenching. A phase shift is measured between the emission hardware-filtered first detector (PMT), and a second excitation detector (cPMT). The 2-PMT system allows a 40-fold increase in measurement resolution and real-time measurement of tissue contraction (FIG. 8). Background interference was filtered using 53.5-kHz and 31.3-kHz two-frequency phase modulation. Measurements were carried out at a 10 Hz sampling rate. Contraction rate and oxygen rhythm frequency were calculated in real-time using a custom-made MATLAB® software imposing Fourier Transformation (FFT) on the evolving kinetic data. Using custom MATLAB code, we calculated the cardiac organoid contraction rates directly from our oxygen reader based on the emission intensity measured in peak-to-peak voltage from the oxygen bead and its displacement using the second PMT system. Cardiac displacement was measured by the embedded oxygen beads inside the cardiac organoids during a contraction. When the beads move at different distances from the focal point, changes in peak-to-peak voltage occurred leading to the detection of the contraction behavior. These data were converted to frequency data using a fast Fourier transform (FFT) by the same MATLAB® code.

### Mitochondrial Membrane Potential Analysis (MMP)

Mitochondrial membrane potential (MMP/ΔΨm) was estimated using JC-1 and TMRE dyes according to the manufacturer's instructions (Invitrogen, USA). Cells were loaded with either 2 μM JC-1 or 2 nM TMRE dye in RPMI supplemented with B27-I for 30 min at 37° C., washed with PBS, and measured in RPMI supplemented with B27-I continuously using an incubated LSM-700 Zeiss microscope at 5% CO2 and 37° C. JC1 measurement was conducted by sequential excitation at 488 nm and 570 nm at a 5 Hz sampling rate. Mitochondrial membrane potential was calculated from the ratio between green (530 nm) and red (590 nm) mean emission intensity in each measurement. TMRE measurement was conducted by excitation at 549 nm at a 10 Hz sampling rate. Mitochondrial membrane potential was calculated from the mean emission intensity at 574 nm. Kinetic analysis was conducted using multiple regions of interest (ROIs) representative of mitochondrial networks. Changes in fluorescent intensity were used to determine oscillation rate and amplitude. Spatial analysis of mitochondrial membrane potential oscillation was done using a custom MATLAB code. Briefly, each continuous mitochondrial membrane potential (MMP) florescent time series micrograph was divided into 10 μm² voxels, and a dominant frequency was identified by FFT for each voxel. The generated frequency matrix was used to create heat map plots representing the spatial analysis of mitochondrial membrane potential oscillation. Heat map plots were generated using ImageJ software (National Institutes of Health, USA).

### MCU Inhibition Studies

For mitochondrial calcium live imaging, different hiPSC-derived cardiomyocytes were cultured in RPMI supplemented with B27 minus insulin, and 0.1% DMSO (control) or 10 M KB-R7943 mesylate (Tocris Bioscieice, USA), Mean mitochondrial calcium was measured using live imaging of Rhod-2AM dye after 0, 30, and 90 seconds of exposure to DMSO (control) or 10 μM MCU inhibitor KB-R7943 mesylate

For simultaneous real-time oxygen, contraction rate, and field potential measurements, cardiac organoids were cultured in RPMI supplemented with B27 minus insulin, 5 ng/ml VEGF and 0.1% DMSO (control) or 10 μM KB-R7943 mesylate. Real-time oxygen, contraction rate, and field potential were measured after 0, 15, and 25 minutes of exposure to DMSO (control) or 10 μM KB-R7943 mesylate.

### Generation of the MCU-KO and Control (NT) CRISPR/Cas9 Plasmids

For MCU-KO, two different sgRNA oligos from the GeCKO v.2 Human CRISPR Knockout Pooled Library (Addgene #1000000048)—MCU HGLibA_28660 and HGLibB_28619—were cloned into the lentiCRISPR v2 plasmid (Addgene #52961). For control (NT), NT1 and NT2 non-targeting/control sgRNA oligos were cloned into lentiCRISPR v2 plasmid.

The sgRNA cloning was performed according to the human GeCKO v.2 system instructions. In brief, the two oligos, comprising each sgRNA insert, were synthesized with BsmBI-compatible ends and were then phosphorylated and annealed in a single session: phosphorylation by T4 PNK (NEB-M0201S) followed by heating to 95° C. for 5 min and controlled cooling to allow annealing. The vector plasmid was digested with BsmBI (FastDigest Esp3I, FD0454, Thermo), de-phosphorylated (FastAP thermosensitive alkaline phosphatase, EF0651, Thermo), and gel extracted (QiaQuick gel extraction, Qiagen). The vector and insert fragments were ligated (T4 DNA ligase, EL0011) and transformed into chemical competent Stbl3 cells (Mix & Go! E. coli Transformation Kit, T3001, Zymo). Proper insertion was verified by Sanger sequencing using the LKO.1 primer.

The plasmids were designated MCU-KO.A (MCU HGLibA_28660), MCU-KO.B (HGLibB_28619), Control-NT1 and Control-NT2 lentiCRISPR v2. Transfection grade plasmid DNA was isolated using the ZymoPURE Plasmid Miniprep Kit (D4209, Zymo Research) according to the manufacturer's instructions. lentiCRISPR v2 plasmid were a kind gift by Nissim Benvenisti lab, HUJI, Jerusalem, Israel.

Generation of MCUKO hiPSC-Derived Cardiomyocytes

The MCU knock-out (KO) was generated by dual lentiviral transduction of MCU-KO.A and MCU-KO.B lentiCRISPR v2 plasmids. Control transduction was similarly done by dual lentiviral transduction of Control-NT1 and Control-NT2 non-targeting lentiCRISPR v2 plasmids. Lentivirus stocks were produced as previously described.

Transduction stocks were prepared by a 1:1 ratio mix of the corresponding lentivirus stocks (MCU-KO.A/B and NT1/2 respectively). hiPSC-CMs were infected at a 50% viral stock dilution for two consecutive sessions of 12 h each. Cell viability was unchanged by the transduction.

### Mitochondrial Calcium Imaging

Mitochondrial calcium uptake within human iPSC-derived cardiomyocytes was measured using Rhod-2AM dye according to the manufacturer's instructions (Abcam, USA). Cells were loaded with 2 M Rhod-2AM in RPMI supplemented with B27-I for 30 min at 37° C., washed with PBS, and incubated with RPMI supplemented with 10 M KB-R7943 or 0.1% DMSO, and B27-I. Measurements were taken continuously using an incubated LSM-700 Zeiss microscope at 5% CO2 and 37° C. Rhod-2AM measurement was conducted by excitation at 552 nm at a 10 Hz sampling rate. Mitochondrial calcium uptake was calculated by the change of the mean emission intensity at 581 nm. The time-lapse micrograph was analyzed using multiple regions of interest (ROIs) and was selected based on Mitotracker staining, producing a distinct area with changes representative to mitochondrial networks. Changes in fluorescent intensity were used to determine oscillation rate and amplitude.

### Ex-Vivo Porcine Studies

The tissue was procured post-mortem from a 70 kg domestic female pig that underwent a laparoscopic liver operation (Ethical approval number MD-21-16533-3) and was euthanized using a lethal dose of potassium chloride. Once the ECG tracing showed a flat line and ETCO2 was zero the pig was pronounced dead, and the left chest was opened lateral to the sternum. The lung was retracted, and the pericardium was incised. The ascending aorta, inferior and superior vena cava were divided as well as the pulmonary veins and the heart was detached from the chest and placed on ice.

The anterior wall of the left ventricle was incised from base to apex and several slices measuring 2-3 mm in length and 1-2 mm thick were obtained using a #10 scalpel blade. Cardiac tissues were further cut into 500±200 μm thick slices and washed thoroughly with 37° C. pre-warmed ex-vivo medium composed of M199 medium, supplemented with 1×insulin-transferrin-selenium (ITS), 10% fetal bovine serum (FBS), and 5 ng/mL vascular endothelial growth factor (VEGF), and 10 mM of 2-[4-(2-hydroxyethyl)piperazin-1-yl]ethanesulfonic acid (HEPES).

The cardiac explants were then embedded with oxygen sensors, placed on the MEAs chip, and transferred to a 37° C. temperature-controlled incubation chamber that contains M199 medium supplemented with 1× ITS, 10% FBS, 5 ng/mL VEGF, and 10 mM HEPES to support long term function, as previously described. The explant was continuously point-stimulated at 2 Hz cycle length. Contraction, field potential, and oxygen were assessed using the integrated metabolic-electro-mechanical sensor chip.

The cardiac explants were paced using a custom-designed, Arduino-controlled, point-stimulator at 2 Hz. The custom design circuit was composed of Arduino MEGA 2560 microcontroller, pulse-width modulation (PWM) to voltage, and digital-to-analog module. The custom code is provided in the relevant availability section.

In the validation assays, simultaneous real-time oxygen, contraction rate, and field potential were measurements in paced cardiac explants, as described above, further supplemented 0.1% DMSO (control), 10 μM blebbistatin, 10 μM mitoxantrone, or 10 μM mitoxantrone and 100 μM metformin (mitoxantrone+metformin).

### Quantification of Metabolic Function

Mitochondrial function was measured using the Seahorse XF Cell Mito Stress Test Kit according to the manufacturer's instructions (Agilent, Santa Clara, CA). Briefly, cardiac organoids or human iPSC-derived cardiomyocytes were seeded on Seahorse XFp mini plates coated with 1% Matrigel at a density of 1 organoid or 3,000 cardiomyocytes per well. Cells were allowed to acclimate for 24 hours. Cultures were then incubated in unbuffered XF Base Medium supplemented with 2 mM Glutamine, 1 mM sodium pyruvate, and 10 mM glucose (pH 7.4) for 1 hour at 37° C. in a non-CO₂ incubator. The inventors measured the basal oxygen consumption rate (OCR) for 30 min and then injected 1 μM oligomycin, a mitochondrial complex V inhibitor that blocks oxidative phosphorylation. The decrease in OCR due to oligomycin treatment was defined as the oxidative phosphorylation rate. 0.5 μM carbonyl cyanide 4-(trifluoromethoxy) phenylhydrazone (FCCP), an uncoupling agent, was added at 60 min to measure maximal mitochondrial activity, and complete inhibition was induced at 90 min using a mixture of 0.5 μM antimycin A and rotenone, mitochondrial complex III and mitochondrial complex I inhibitors. The results were normalized to the total cell number per well. The 2D hiPSC-CM remained at approximately the seeding numbers by the time of assay, as determined by Hoechst staining post the Mito stress assay. In brief, two separate fields per well were imaged on an Olympus X81 microscope, the raw images exported and cell nuclei were counted on Cell Profiler as primary objects.

For cell counting of cardiac organoids, genomic DNA (gDNA) was extracted from single organoids or known quantities of 2D hiPSC-CM cells using the Quick-DNA miniprep plus kit (D3024, Zymo Research) according to the manufacturer's instructions. gDNA concentration and total gDNA extracted per sample were measured using a Nanodrop 1000. To find the number of cardiomyocytes per organoid, we utilized qPCR with human-specific primers, amplifying a 156 bp region of gene EDEM1. The qPCR was performed as previously described. The template from multiple single organoids and 10⁵ iPSC-CM 2D cells was two-fold diluted from 10 ng to 0.625ng per reaction and all samples/dilutions were assessed in quadruplicates. The average Ct values per template quantity were plotted and samples with comparable slopes/efficiencies were further assessed. Considering a similar qPCR efficiency for the latter, the ΔCt between organoid and 2D cardiac cells was used to calculate the percentage of human cardiac cells in each organoid. Cardiac cell numbers per organoid were estimated by comparing the total gDNA quantity extracted from 10⁵ iPSC-CM 2D cells to the total gDNA extracted from each organoid and applying the qPCR-estimated percentage of cardiac cells. The human cardiomyocytes in each organoid are significantly more oxidative than the rat endothelial cells, thus, they are considered the sole contributors to the assay results.

### Metabolic Measurements and ATP Production

Amperometric 4-analytes sensors (B.LV5) were purchased from Innovative Sensor Technology (IST, Switzerland). Measurements were carried out and calibrated to sensitivity decrease by an on-chip potentiostat (IST, Switzerland). Oxygen, glucose, lactate, and glutamine fluxes were measured by calculating the change in metabolite concentration over time. Glutamine measurement allowed us to calculate lipid metabolism fluxes. Oxidative phosphorylation flux was calculated by dividing the oxygen uptake rate by six. The inventors estimated 32 ATP molecules generated by the complete oxidation of glucose. Glycolysis flux was calculated by dividing the lactate production rate by two, with the maximal rate defined by glucose uptake rate minus the oxidative phosphorylation flux. ATP production in glycolysis was estimated to be two molecules per molecule of glucose. Glutaminolysis was calculated directly by glutamine uptake. ATP production in glutaminolysis was estimated to be three molecules per molecule of lactate generated.

### Quantification RT-PCR

Total RNA isolation from hiPS-derived CM or cardiac organoids was performed using NucleoSpin RNA II kit (Macherey-Nagel, Germany) according to the manufacturer's instructions. RNA concentration and purity were determined using a NanoDrop ND-1000 spectrophotometer (Thermo Fisher Scientific, USA). cDNA synthesis was performed using qScript™ cDNA Synthesis (Quanta BioSciences) according to the manufacturer's instructions. 1 g of purified RNA was used for each reaction.

qRT-PCR was performed using KAPA SYBR FAST (Kapa Biosystems) on Applied Biosystems™ QuantStudio™ 5 Real-Time PCR System. Data analysis was done by normalizing Ct values of target genes on that of the RPL32 gene, and data were presented as relative quantification (RQ) values. The primers used were (5′ to 3′): EDEM1-F-GGCCCCCGCGCTTTAAAATA (SEQ ID NO: 1); EDEM1-R-GGAAAGCGCTGGTAGAAGCC (SEQ ID NO: 2); MCU—F-CACACAGTTTGGCATTTTGG (SEQ ID NO: 3); and MCU—R-CGTGACTTTTTGGCTCCTTT (SEQ ID NO: 4).

### Processing, Analysis, and Graphic Display of Genomic Data

R studio (www.rstudio.com/) was used to perform principal component analysis (PCA; prcomp package), scatter plots, and volcano plots (ggplot package).

Hierarchical clustering, heat maps, correlation plots, and similarity matrices were created in Morpheus. Gene ontology enrichment analyses and clustering were performed using DAVID Informatics Resources 6.7 and PANTHER Classification System. Metabolic network maps were created using McGill's Network Analyst Tool using the KEGG database.

### Scanning Electron Microscopy

Organoids samples were precoated with an Au—Pd nanolayer using an SC7640 Sputter. SEM imaging was performed using the FEI Sirion High-Resolution Scanning Electron Microscope (HR SEM, Holland). Images were taken at secondary electron (SE) detection with an accelerating voltage of 5 kV, a spot size of 4.0 and at a 5.3 mm working distance using high-resolution mode. TSL-EDAX (EDAX, USA) system was mounted for Electron Back Scattered Diffraction (EBSD).

### Quantification and Statistical Analysis

All experiments were performed in at least 3 biological repeats. Measurements were performed in either technical triplicates or quadruplets, images were analyzed with 5 or more fields of view; Graphs show mean±SEM. Pairwise comparisons were performed using Student's t-test; the Mann-Whitney U test was used when the distribution could not be determined to be normal; False discovery rate (FDR) correction was used to adjust for multiple comparisons and RNA seq comparisons. * Indicates p<0.05, ** indicates p<0.01, *** indicates p<0.001, unless denoted otherwise.

### Software Resources

Custom analysis software is available at: github.com/mohammadghosheh95/Heart-on-a-Chip.

### Statistical Analysis

Experiments were repeated 3 times with triplicate samples for each experimental condition unless stated otherwise. Data from representative experiments are presented, and similar trends were seen in multiple trials. A parametric two-tailed Student's t-test was used for calculating significant differences between groups. All error bars represent ±SE unless otherwise noted.

### Example 1

### Generation of Vascularized Multi-Chambered Cardiac Organoids by Anisotropic Stress

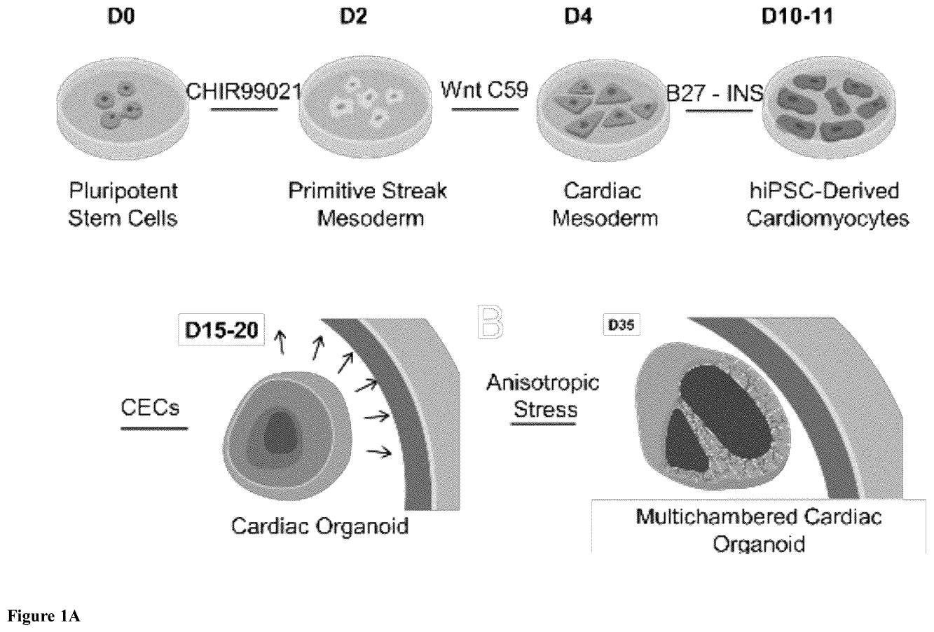

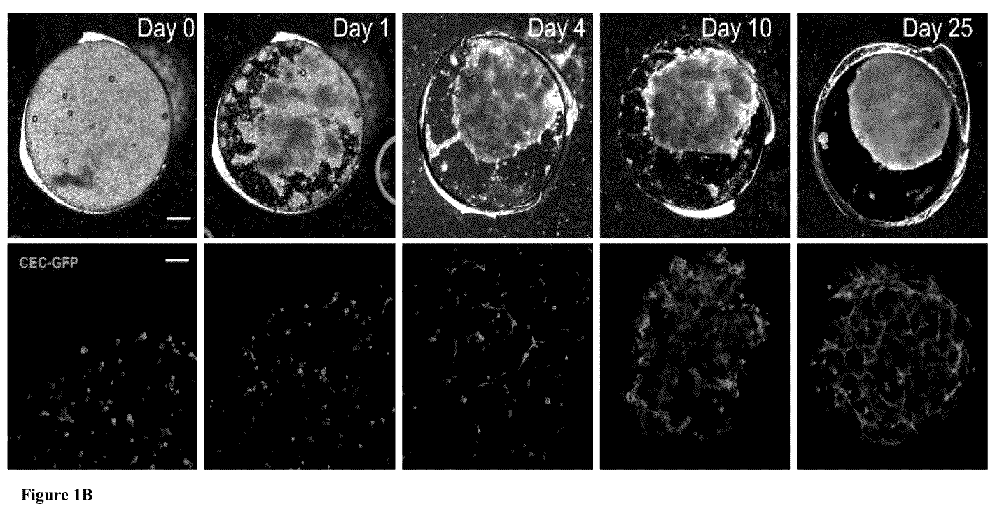

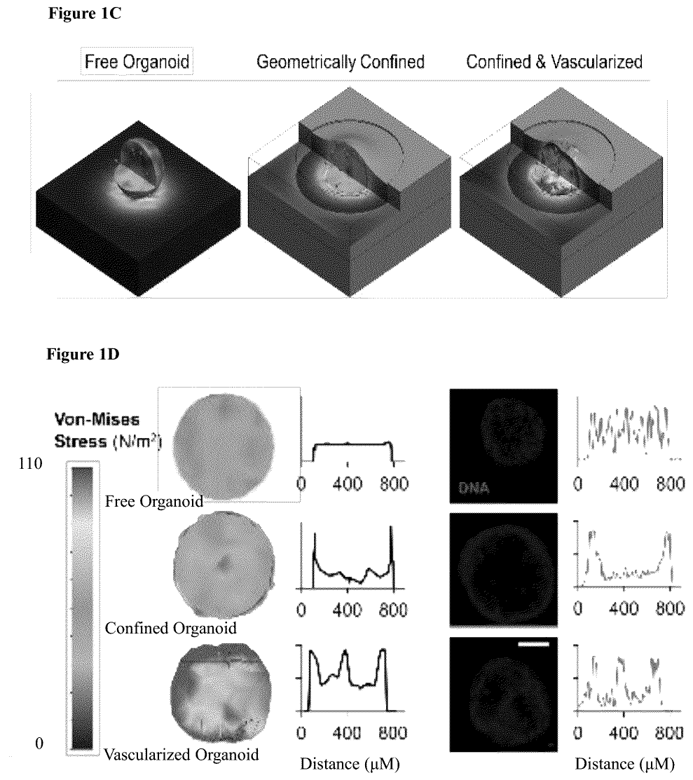

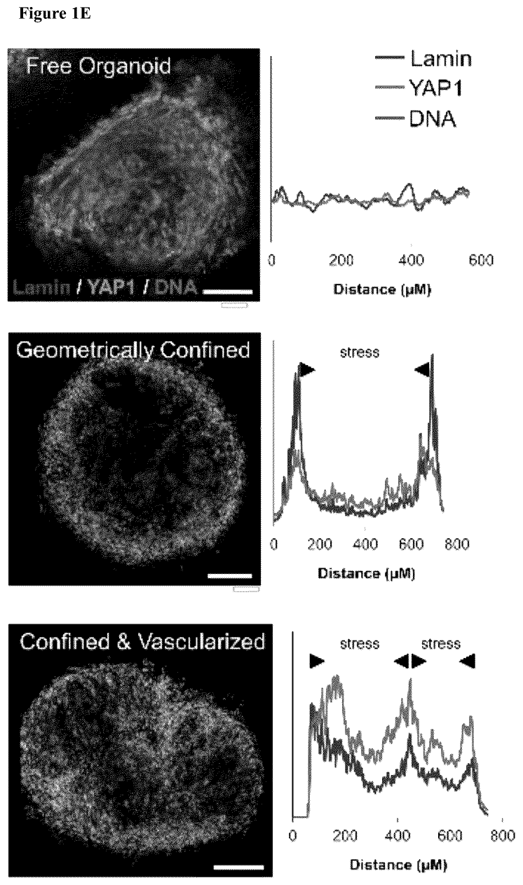

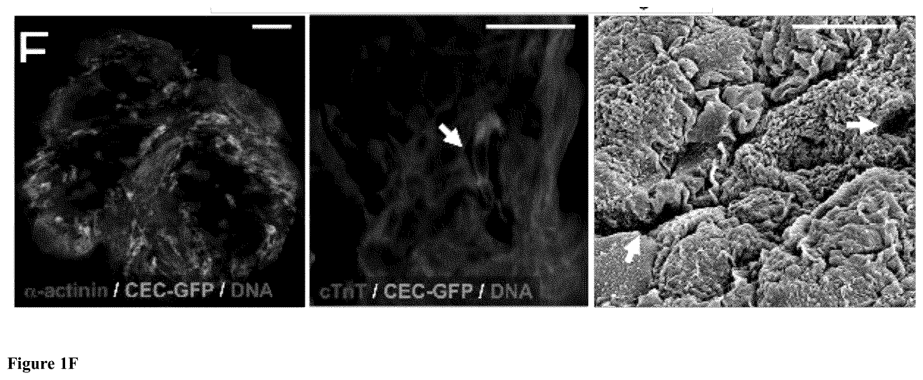

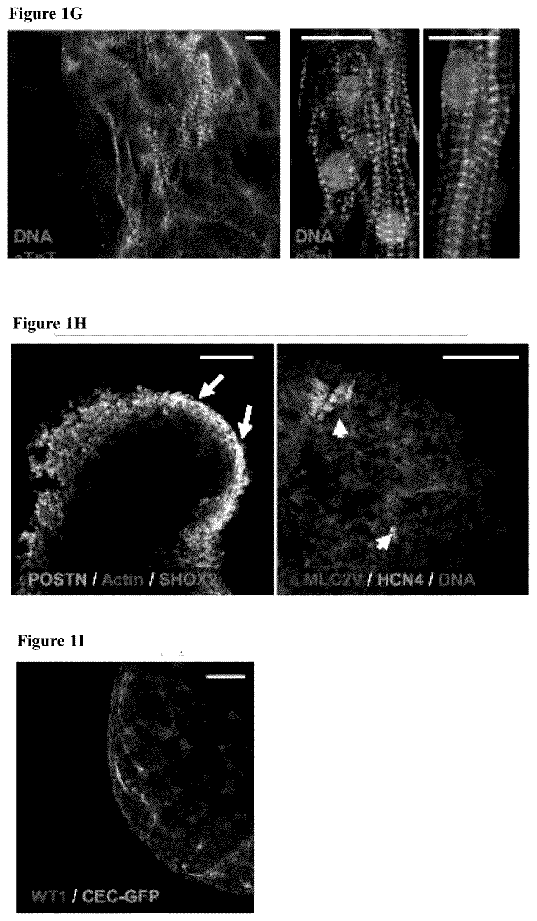

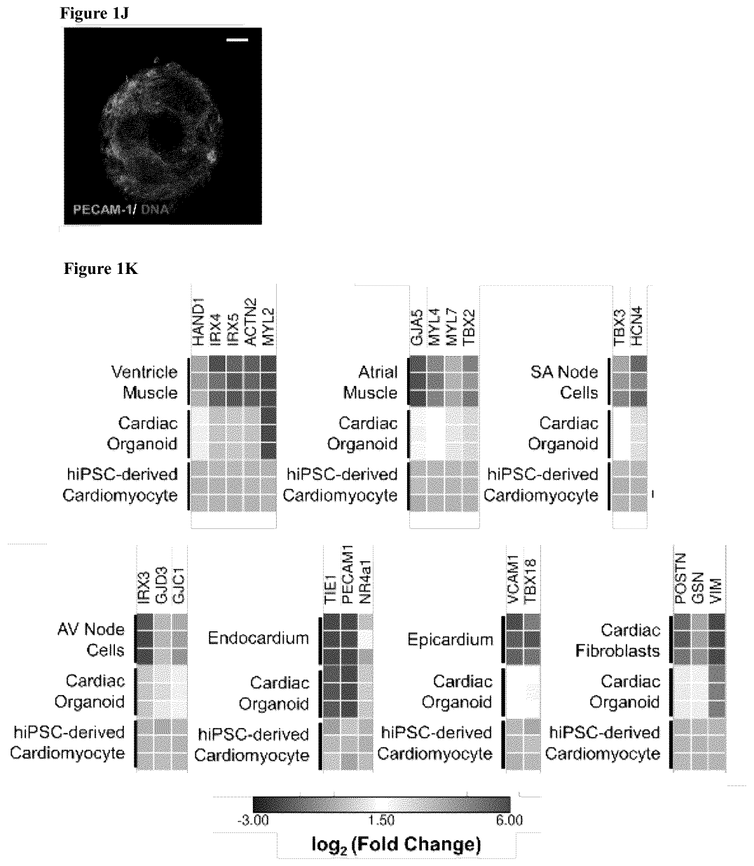

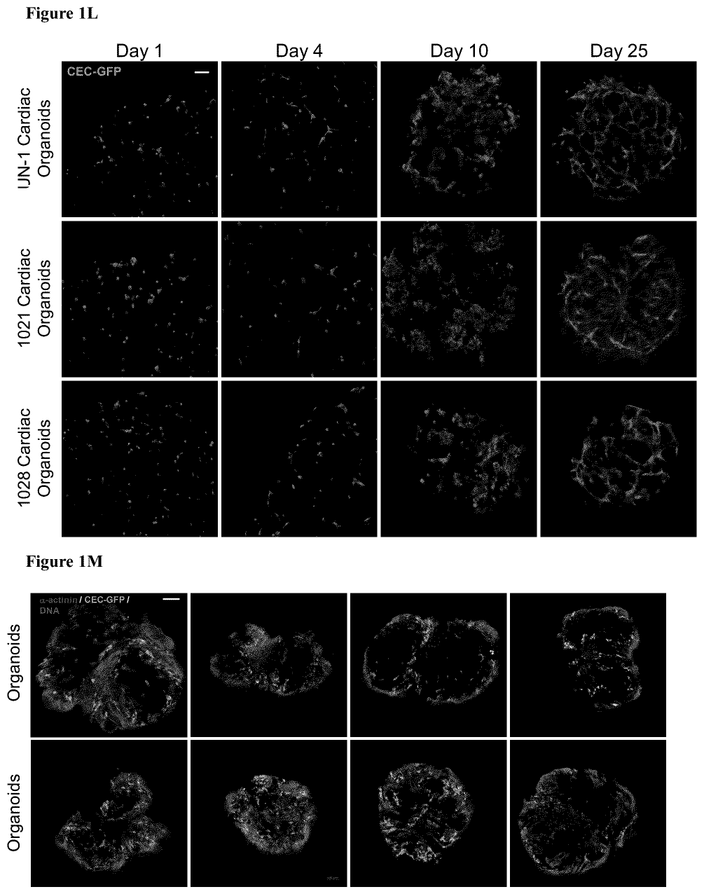

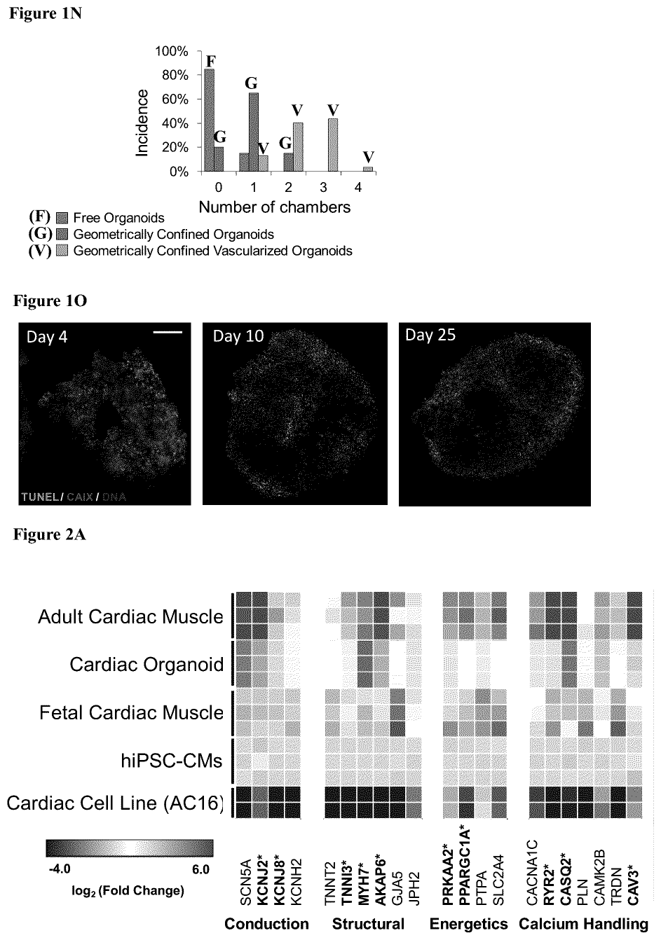

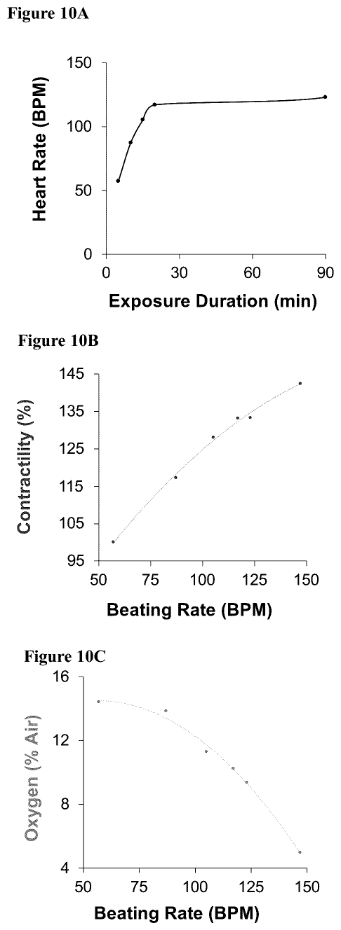

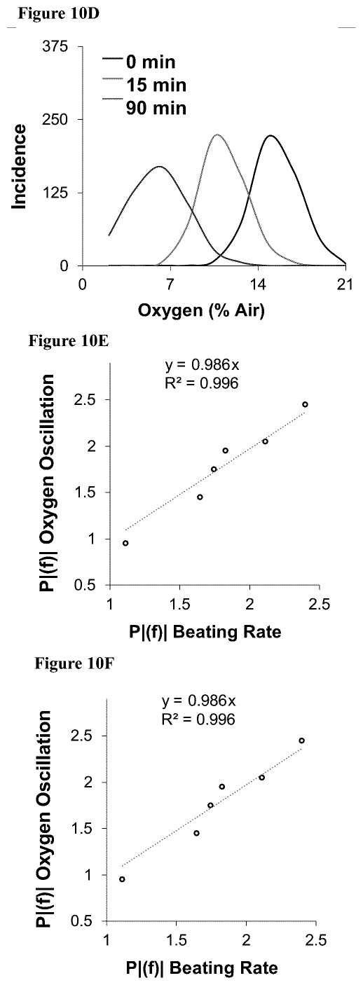

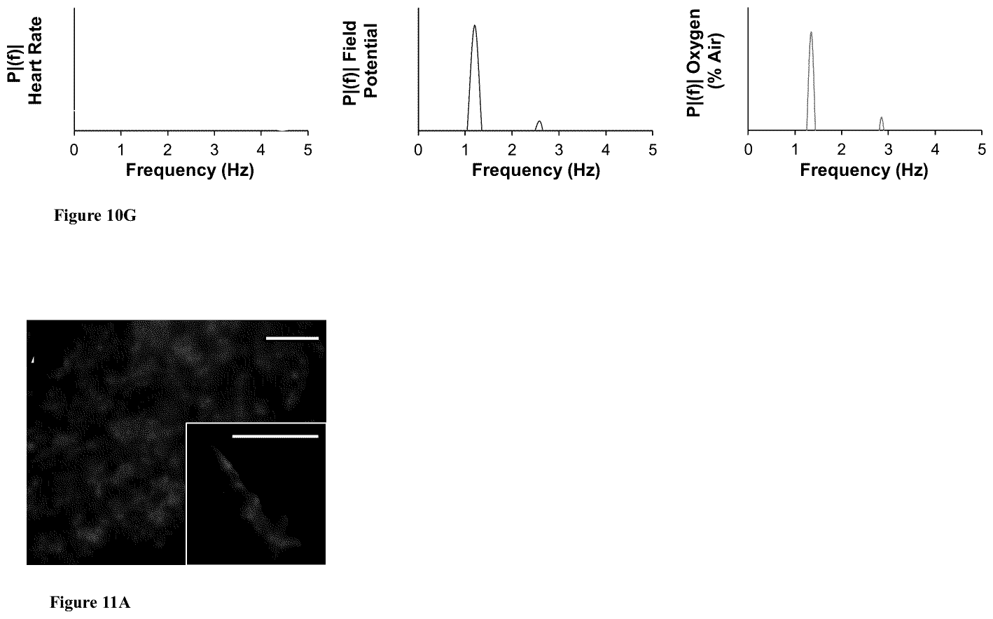

Developmental studies showed that complex cardiac partitioning only occurs following the development of the cardiac vasculature. To mimic this developmental step, the inventors seeded a mixture of hiPSC-derived cardiomyocytes (hiPSC-CMs) and cardiac microvascular endothelial cells in basement membrane matrix into microwells (FIGS. 1A-1B). The tissue contracted into a single mass in 4 days and started beating after 10 days. Cardiac organoids were cultured over the next weeks acquiring smooth exterior and synchronized behavior following 25 days of culture (FIG. 1B). GFP-expressing endothelial cells reveal that networks form after day 10, followed by the development of a complex circumferentially-aligned vascular network after 25 days (FIG. 1B, 1L). Confocal microscopy of the cardiac organoids showed a complex structure supporting 2-3 chambers in both UN-1 and ACS-1021 derived organoids (FIG. 1M-1N). Immunostaining for TUNEL and Carbonic anhydrase IX (CAIX), an endogenous marker of hypoxia, indicates that the formation of the chamber is not a result of hypoxia or apoptosis during the formation of the organoid (FIG. 10).

To better understand this complex organization, the inventors modeled the von Mises stress distribution and Gaussian displacement of cardiac organoids undergoing contraction in different conditions (FIGS. 1C-1D, and Materials and Methods). Stress-free cardiac organoids without geomatic confinement or vascular structures resulted in homogenous stress distribution associated with the formation of a solid spheroid. While geometric confinement in the absence of vascular structures (i.e., without the addition of endothelial cells) produced isotropic stress gradients, it resulted in a single chamber (FIGS. 1C-1D, and 7A-7B). However, incorporation of vascular-like structures within the mass due to the inclusion of endothelial cells, produced anisotropic stress gradients, resulting in multiple low-stress regions where the tissue separated into multiple chambers (FIGS. 1C-1D, 1M-1N and 7A-7B).

To validate the results of the finite element model the inventors used confocal microscopy to analyze the distribution of mechano-transduction markers lamin A/C (LMNA) and YAP1 in different conditions (FIG. 1E). Free organoids showed homogenous expression of LMNA and YAP1 marking uniform stress distribution. Geometric confinement resulted in a circumferential ring of high stress marked by LMNA a gradient of YAP1 surrounding the central cavity. Incorporation of vascular-like structures within the mass, produced anisotropic stress gradients, resulting in high-stress regions marked by LMNA, separating multiple chambers. Mechanosensor expression patterns correspond to the modeled stress patterns (FIG. 1D-E, FIG. 7C).

Confocal microscopy revealed a robust formation of multi-chambered cardiac organoids (FIG. 7C), composed of circumferentially aligned cardiomyocytes enveloping multiple hollow chambers (FIG. 1E, 1M). Immunostaining for α-actinin and cardiac troponins revealed the organoids are composed of elongated cardiomyocytes with an organized sarcomeric pattern (FIG. 1G). Confocal and scanning electron microscopy showed patent endothelial capillaries threaded throughout the chamber walls (FIG. 1F). Structural analysis shows that POSTN+ cardiac fibroblast-like cells weave into the cardiomyocyte layer, decorated by pacemaker-like cell clusters positive to HCN4 and SHOX2 (FIG. 1H). Confocal microscopy revealed an outer shell of epicardium-like cells positive to WT1 and TBX18 (FIG. 1I). Cardiac chambers appear to be partly layered with PECAM-1 positive endocardial-like cells (FIG. 1J). These structural findings were further validated by RNA sequencing of the cardiac organoids, compared human tissue, and hiPSC-CMs from two-dimensional cultures. Cardiac organoids showing expression signatures associated with pacemaker (sinoatrial and atrioventricular nodes), endocardium, and epicardium cells, as well as cardiac fibroblasts (FIG. 1K).

### Example 2

### Functional Characterization of Multi-Chambered Cardiac Organoids

To assess the cardiac maturity of the model an RNA-Seq analysis was carried out on the multi-chambered cardiac organoids, as compared to hiPSC-CMs grown in two dimensions, as well as adult and fetal cardiac tissues (FIG. 2A-B). Transcriptomic analysis of the multi-chambered cardiac organoids showed functional gene expression correlated to adult rather than fetal cardiac muscle (FIG. 2A). Genes involved in cardiac conduction (KCNJ2, KCNJ8), ultrastructure (TMNI3, MYH7, AKAP6), energetics (PPKAA2, PGC1A), and calcium handling (RAR2, CASQ2, CAV3) were differentially up-regulated in cardiac organoids compared to both hiPSC-CMs (2D culture) as well as fetal cardiac muscle (FIG. 2A). Principle component analysis (PCA) of 513 genes that were differentially expressed between cardiac organoids and hiPSC-CMs (2D) distinguished between adult and fetal cardiac expression, clustering the cardiac organoids with the adult tissue (FIG. 2B).

Vascularized cardiac organoids exhibited a spontaneous synchronized beating of 66±5 beats per minute (bpm) as well as a physiological response to drugs. Epinephrine stimulation increased the beating rate to 88±7 bpm from the baseline value (n=6, p<0.001) and contraction amplitude by 18% (n=6, p<0.01). In contrast, antiarrhythmic medication amiodarone decreased the contraction rate to 52±4 bpm from the baseline value (n=6, p<0.01) and contraction amplitude by 28% (n=6, p<0.01; FIG. 2C). Importantly, the herein disclosed cardiac organoids showed basal respiration and oxidative phosphorylation that are 35% and 85% higher than hiPSC-CMs, respectively (n=3, p<0.05; FIG. 2D). Mitochondrial maximal capacity, an indicator of metabolic maturity, was 2-fold higher than basal respiration (FIG. 2D).

The metabolic dynamics of cardiac organoid contraction and relaxation were tracked using embedded oxygen sensing beads in the organoid (FIGS. 2E-F, and 3A) as well as electrochemical sensors to monitor glucose, lactate, and glutamine. Metabolic flux balance analysis (see Materials and Methods) demonstrates the dominance of fatty acid oxidation over other metabolic pathways, characteristic of mature cardiac muscle (FIG. 2F). This metabolic analysis showed that changes in interstitial oxygen occur during the cardiac cycle, in sub-second resolution (FIG. 2F).

### Example 3

### Embedded Sensors Link Respiration to Cardiac Electromechanical Rhythms

Cardiac rhythms occur at a sub-second resolution, too quickly for most metabolic sensors and register changes. To address this issue, the inventors designed a dual-photomultiplier (PMT) sensing platform that enables real-time active background noise reduction, allowing phosphorescence measurements from oxygen sensors embedded in cardiac organoids with lower than 100 milliseconds resolution (FIGS. 3A-3E; and FIG. 8A). Phase shift measurement was used to track oxygen consumption while sensor reflection of the excitation wavelength allowed accurate measurement of tissue displacement (FIG. 3B; and FIG. 8A-B). Integration of this new system with nano-fabricated optically transparent microwell-enclosed microelectrode array (MEA, FIG. 3C-3E and FIG. 8C-D) uniquely allows simultaneous measurements of cardiac contraction (FIG. 3F), field potentials (FIG. 3G), and oxygen consumption (FIG. 3H) in real-time. Tracking the embedded optical sensors showed organoids contracted by 9.3±1.1% along their axis during the beating cycle (FIG. 3F and FIG. 9A). The organoid contraction was analyzed by Fast Fourier transform (FFT) showing a dominant frequency of 0.94±0.03 Hz indicating 58±2 bpm (n=9; FIG. 3F and FIG. 9B). The recorded electrical field potential of each organoid was filtered and amplified in real-time showing a similar dominant frequency at 0.99±0.03 Hz (n=9; FIG. 3G and FIG. 9A-9D).

While metabolic fluxes are thought to change in timescales ranging from minutes to hours, the herein disclosed real-time optical measurements of interstitial oxygen concentration in cardiac organoids showed a rapid oscillation, with a dominant frequency of 0.92±0.03 Hz (n=9; FIG. 3H and FIG. 9A-9D) indicating a link between respiration and the organoid electromechanical cycle. Similar oscillations were also recorded in ACS-1021, and ACS-1028 cardiac organoids (FIG. 9A).

### Example 4

### Unraveling the Electro-Metabolic Coupling of Human Cardiac Tissue

Epinephrine, released during physiological fight-or-flight response, rapidly increases contraction rate and contractility and on longer timescales, cardiac glucose metabolism. To study the dynamics of this metabolic response in sudden stress the inventors exposed cardiac organoids to 100 μM epinephrine and tracked their mechano-metabolic response in real-time. As expected, organoid contraction frequency doubled from 0.95 to 1.95 Hz (n=3, p<0.001; FIG. 3I) while its contraction increased by 33% (n=3, p<0.001) within 15 minutes of stimulation. Interstitial oxygen concentrations again showed matching oscillations, with frequency similarly doubling from 1.11 to 1.83 Hz within 15 minutes of stimulation (n=3, p<0.001; FIG. 3J). Interestingly, the increased organoid contraction was coupled to the amplitude of interstitial oxygen oscillation, suggesting coordination between the cardiac function and oxygen consumption (FIG. 10A-F).

To determine whether the respiratory cycles were driven by mechanical forces or depolarization the inventors treated the cardiac organoids with blebbistatin, an excitation-contraction decoupling compound, and tetrodotoxin (TTX) a Naᵥ channel inhibitor. Blebbistatin inhibits myosin II, thereby preventing myosin contraction without affecting action potential in cardiac cells. Blebbistatin treatment blocked cardiac contraction, and sensor movement but did not affect the field potential or the oxygen oscillation frequency and intensity (FIG. 3K and FIG. 10G). Tetrodotoxin treatment eliminated field potential generation, mechanical contraction, and oxygen oscillation in cardiac organoids, within seconds of effect onset (FIG. 3L). These results demonstrate that oxygen oscillations are coupled to the electrical rather than the mechanical activity of the cells.

### Example 5

Dynamics of Mitochondrial Function in Beating hiPSC-Derived Cardiomyocytes

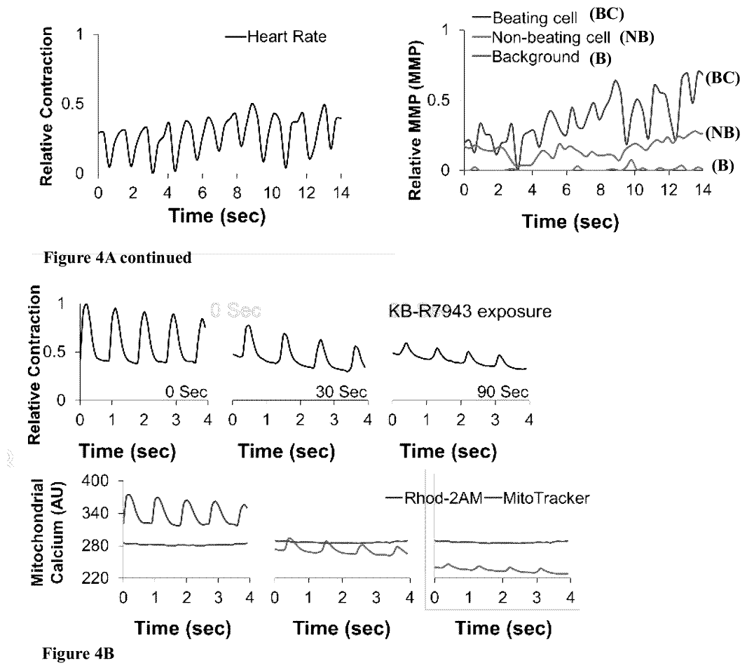

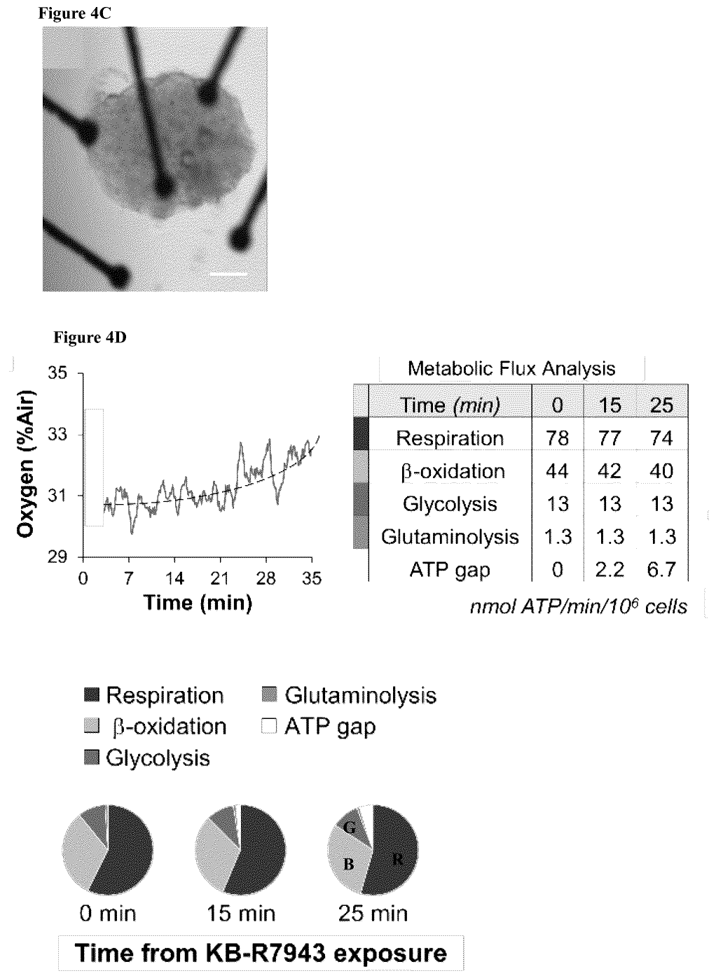

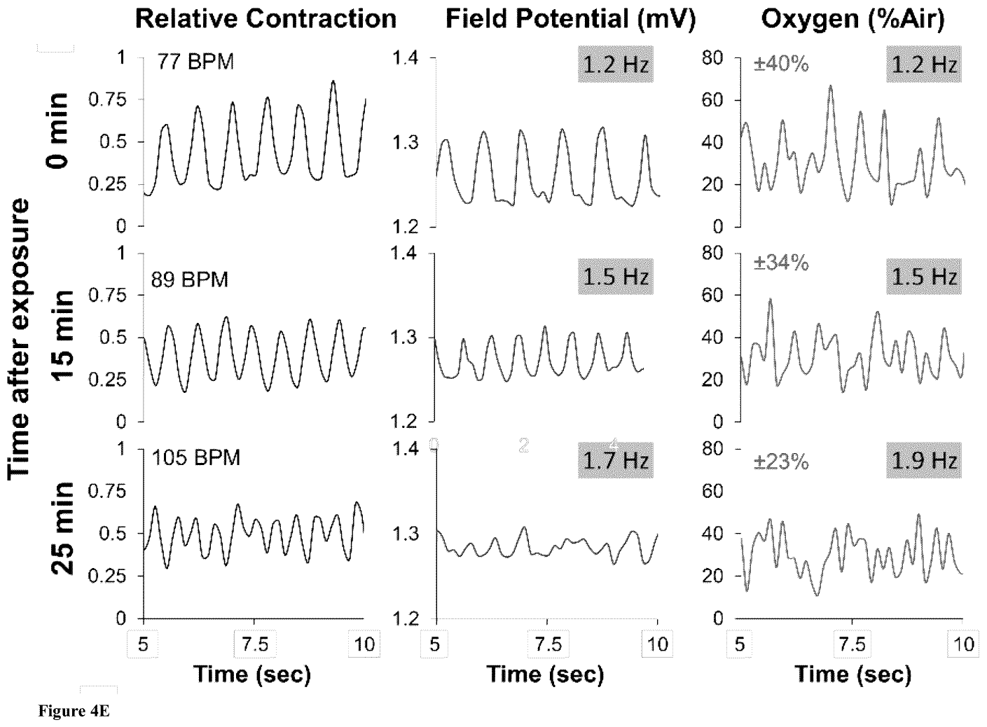

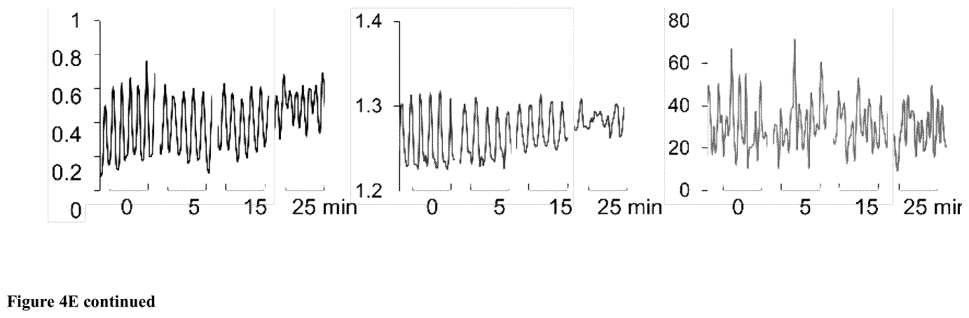

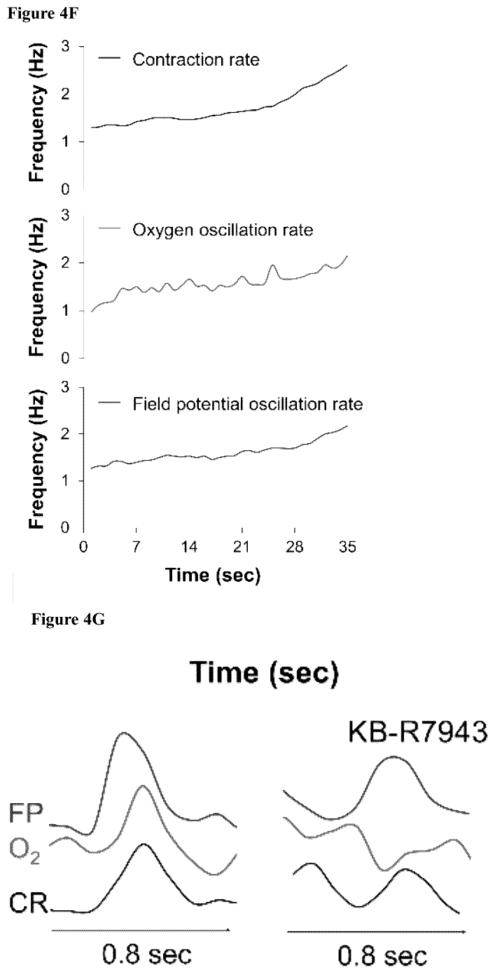

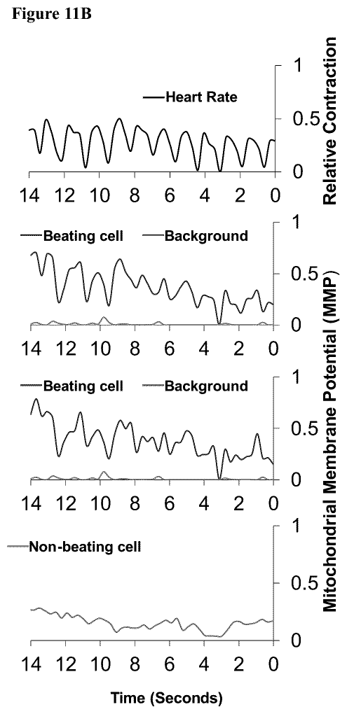

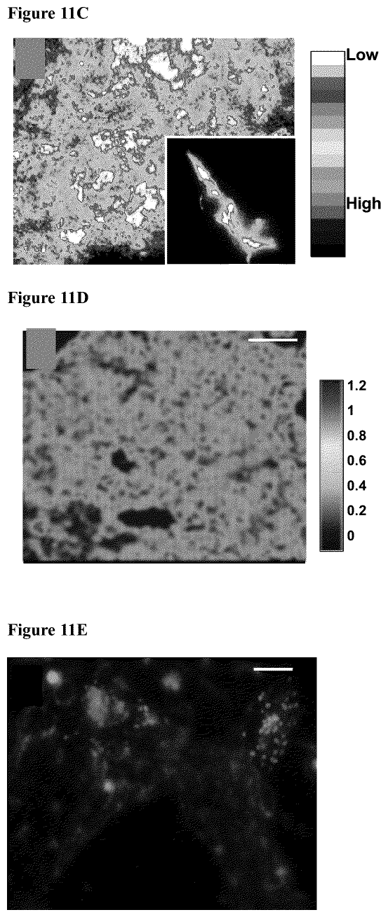

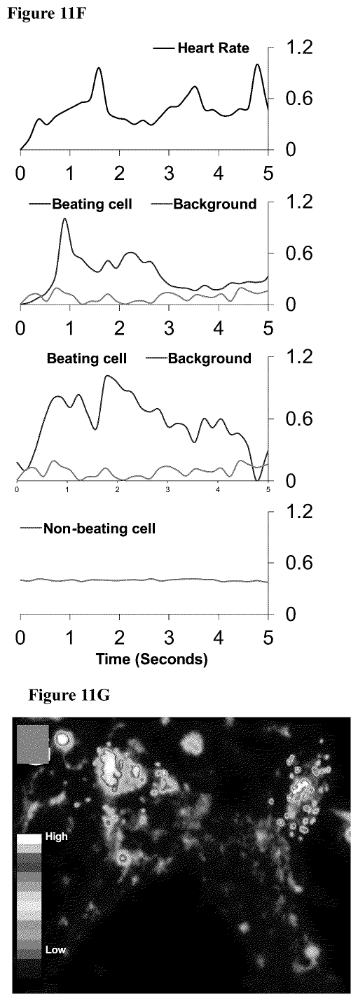

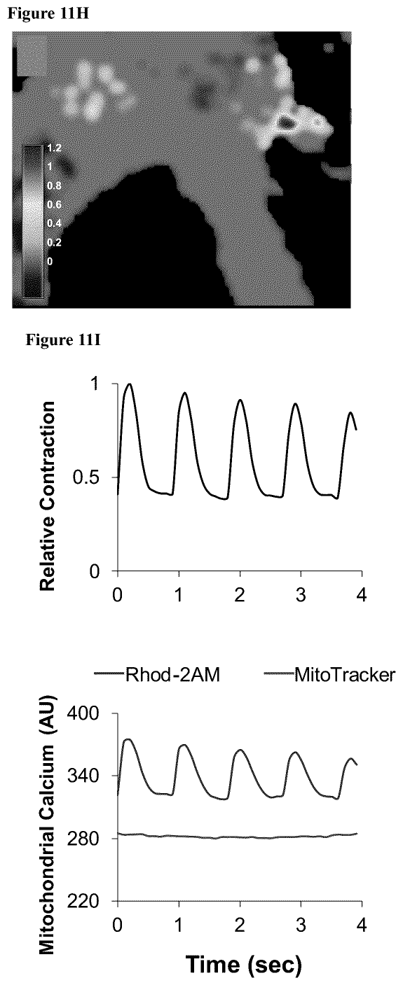

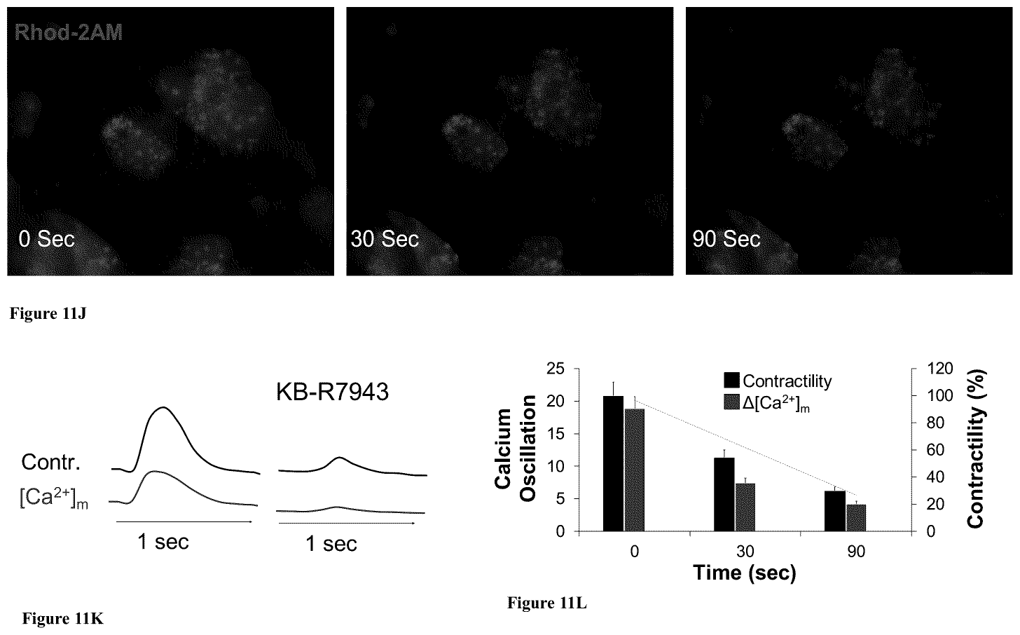

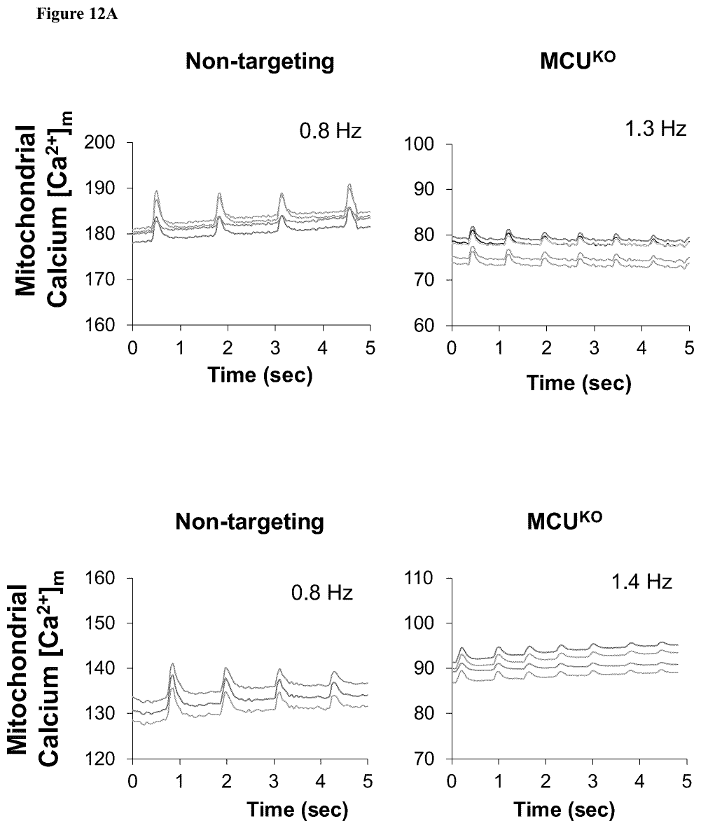

Rapid metabolic changes, on the millisecond scale, are more likely to be controlled by ion fluxes than enzymatic cascades. Mitochondrial membrane potential (MMP/ΔΨm) is mitochondrial calcium ([Ca²⁺]m)-dependent indicator of respiratory function. Mitochondrial membrane potential was measured in beating hiPSC-derived cardiomyocytes and non-beating cells in 2D culture by live-cell imaging using TMRE (FIG. 4A). Beating cells showed MMP oscillations precisely correlated to cardiomyocyte contraction (FIG. 4A and FIG. 11A-H). Non-beating cells in the same field did not show oscillating mitochondrial membrane potential and on average had a 3-fold lower MMP than spontaneously contracting cells (n=5, p<0.001; FIG. 4A and FIG. 11A-H). Rhod-2AM is a mitochondrial-specific dye whose fluorescence is dependent on Ca⁺² binding. Live imaging of hiPSC-derived cardiomyocytes in 2D culture (see Materials and methods) showed rapid oscillation in mitochondrial calcium [Ca²⁺]ₘ in beating cells, precisely correlated to the contraction frequency of the cells (FIG. 4B, and FIG. 11I) Together, these data strongly suggest that changes in [Ca²⁺]m during the contraction cycle, produce the rapid oscillation in oxygen consumption.

To examine the role of [Ca²⁺]ₘ on cardiomyocytes rhythmic function, the inventors exposed the cells to KB-R7943, a pharmacological inhibitor of the mitochondrial calcium uniporter (MCU; FIG. 4B and FIG. 11J). Short term exposure to MCU inhibitor did not significantly affect cardiomyocyte contraction frequency (FIG. 4B), but dramatically reduced contraction magnitude by 45% and 70% in 30 and 90 seconds, respectively (n=4, p<0.01; FIG. 4B and FIG. 11J-L). Parallel measurements of mitochondrial calcium following KB-R7943 exposure showed no significant difference in [Ca²⁺]ₘ concentration, but the magnitude of [Ca²⁺]ₘ oscillations decreased by 60% and 78% in 30 and 90 seconds, respectively (n=4, p<0.001; FIG. 11L). These data suggest that the magnitude of cardiomyocyte contraction is dependent on the efficient oscillation of mitochondrial calcium.

### Example 6

### Real-Time Effect of MCU Inhibition in Vascularized Cardiac Organoids

The current results suggest that mitochondrial activity oscillates in spontaneously beating cardiomyocytes due to the rhythms of mitochondrial calcium. To study this behavior in a physiological model the inventors exposed cardiac organoids cultured on the herein disclosed sensor-integrated platform to the MCU inhibitor, KB-R7943 (FIG. 4C). MCU inhibition induced a progressive decrease in the oxygen uptake of the cardiac organoid (FIG. 4D). Metabolic flux balance analysis showed a gradual decrease in respiration and fatty acid oxidation leading to a decrease in ATP generation within 20 minutes of exposure to the MCU inhibitor (FIG. 4D).

Analysis of cardiac organoid metabolic activity showed that the magnitude of oxygen oscillations significantly and progressively decreased from the moment of exposure of KB-R7943 (n=9; FIG. 4E). Concomitantly, the magnitude of the cardiac organoid contraction and its depolarization showed a comparable and parallel decrease, following MCU inhibition (FIG. 4E). These data indicate loss of function driven by the observed mitochondrial disruption. However, while the metabolic activity of the cardiac organoids decreased, cardiac contraction frequency, oxygen oscillation rate, and the frequency of the field potential oscillations significantly increased following MCU inhibition (FIG. 4F). All three rates showed a 2.5-fold increase within 35 minutes of exposure indicating arrhythmic behavior (n=9; FIG. 4F). This arrhythmogenic behavior was associated with loss of synchronization shifting oxygen oscillation away from the contraction cycle (FIG. 4G). Taken together, the herein disclosed data suggest that MCU inhibition severs an electro-mitochondrial link, leading to arrhythmogenic behavior.

### Example 7

### CRISPR/Cas9 Knockout of MCU Disrupts Electro-Mitochondrial Coupling and Induces Arrhythmic Behavior.

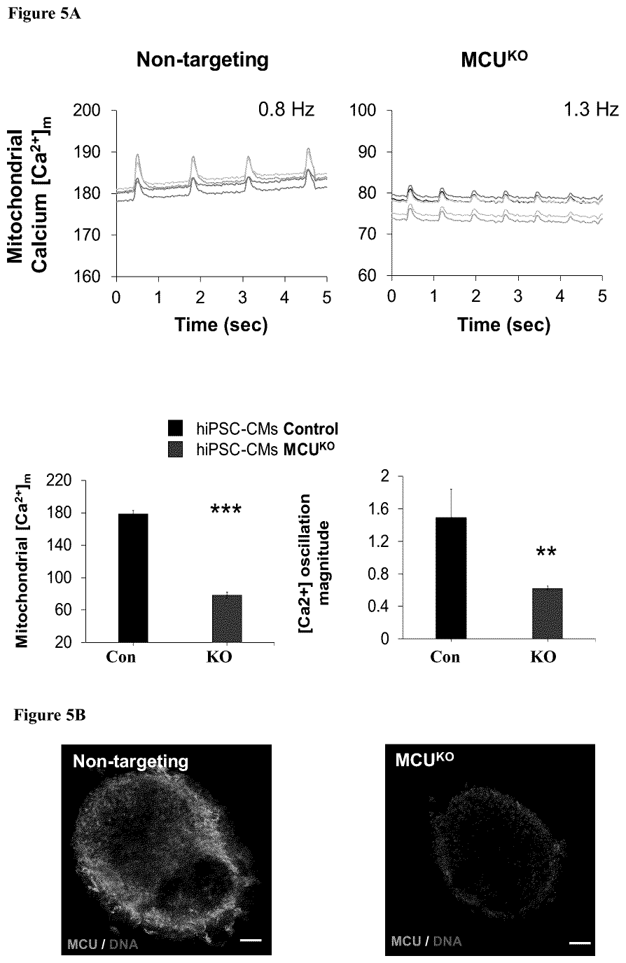

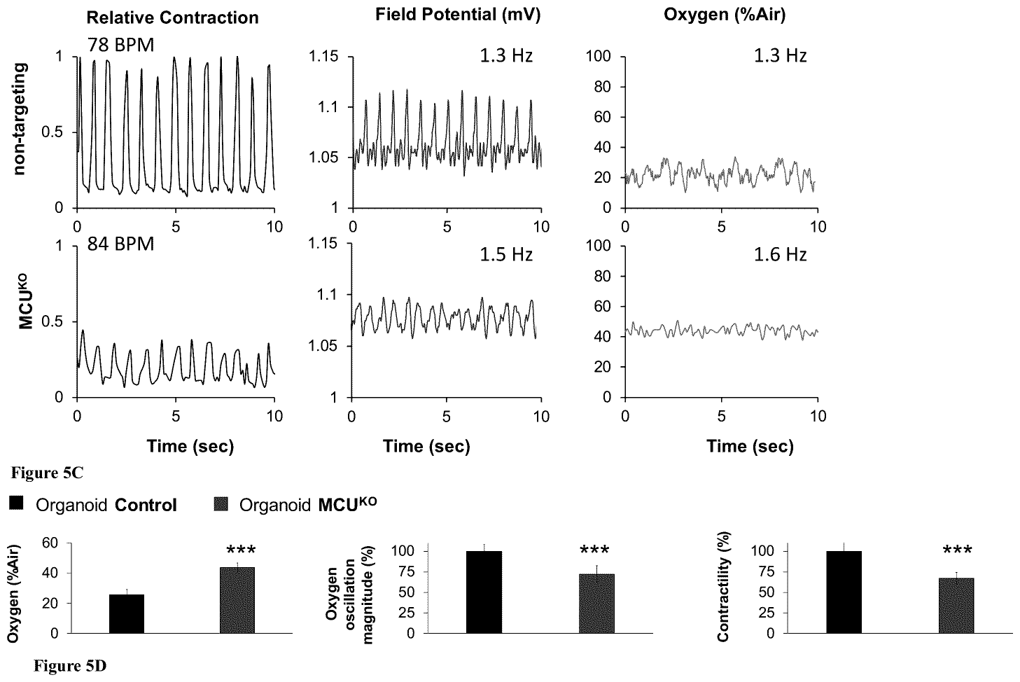

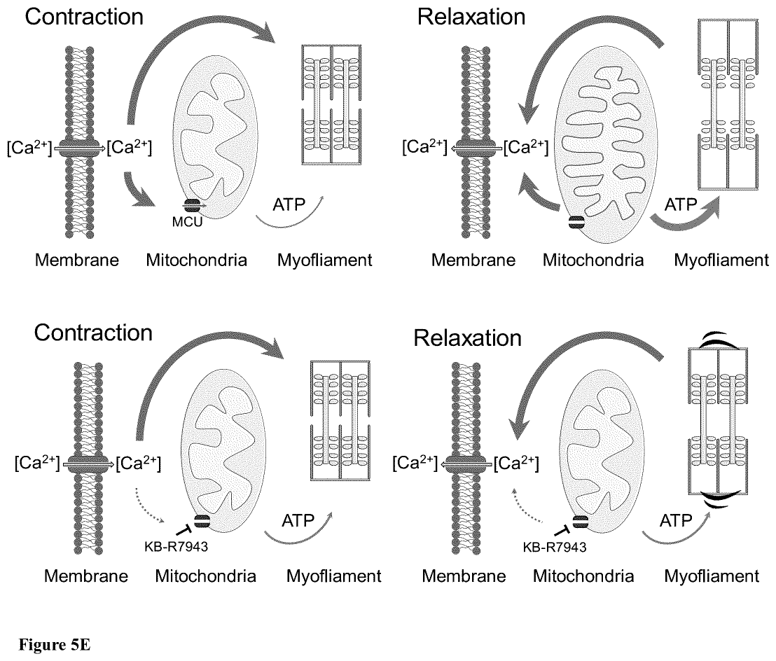

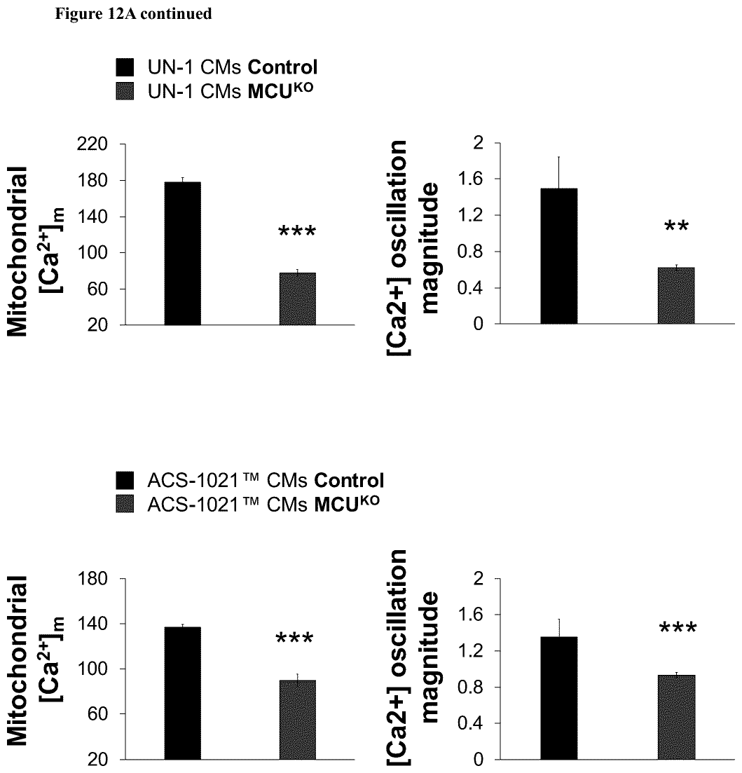

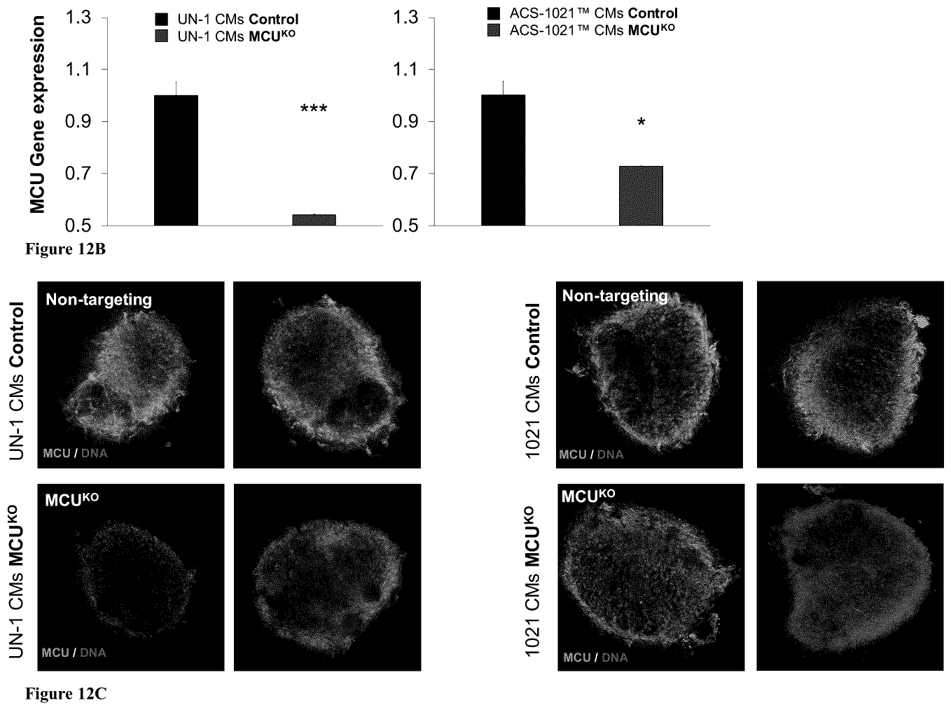

To further validate the proposed mechanism, a CRISPR/Cas9 knockout of MCU was produced in hiPSC-CMs in 2D culture (see Materials and methods). As antibiotic selection interferes with cardiac maturation and survival, the analysis relies on mixed cultures. Live imaging of hiPSC-CMs (2D) showed conventional oscillation of mitochondrial calcium [Ca²⁺]ₘ in cells transfected with non-targeting sgRNA exhibiting a dominant frequency of 0.8 Hz (n=7; FIG. 5A and FIG. 12A). In contrast, MCU knockout (MCU^{KO}) cells showed a 50% decrease in [Ca²⁺]ₘ while the oscillation rate increased to 1.3 Hz (n=7, p<0.01; FIG. 5A and FIG. 12A).

Next, the non-homogenous MCU^{KO} cardiomyocytes were used to form a chimeric cardiac organoid (FIG. 5B). RT-qPCR and confocal microscopy demonstrated a marked reduction in MCU expression on mRNA and protein levels (FIG. 5B, and FIG. 12B-C). Contraction, field potential, and interstitial oxygen were measured using the integrated metabolic-electro-mechanical sensor chip in these cardiac organoids. Cardiac organoids composed of MCU^{KO} cells had lower contractility and lower magnitude of oxygen oscillation (n=9, p<0.001; FIG. 5D). MCU^{KO} organoids showed increased beating frequency and irregular field potential indicating arrhythmogenic behavior (FIG. 5C). Taken together, these findings suggest that electro-mitochondrial coupling is driven by [Ca²⁺]ₘ oscillations in cardiomyocytes (FIG. 5D). This coordination allows the muscle to prepare for the spike in energy demand needed for mechanical contraction (FIG. 5D).

### Example 8

### Metformin Reverses Mitoxantrone-Induced Arrhythmia

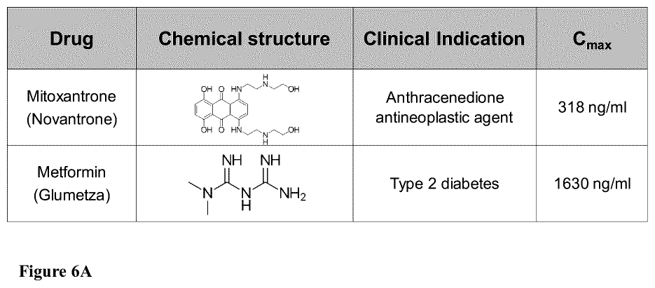

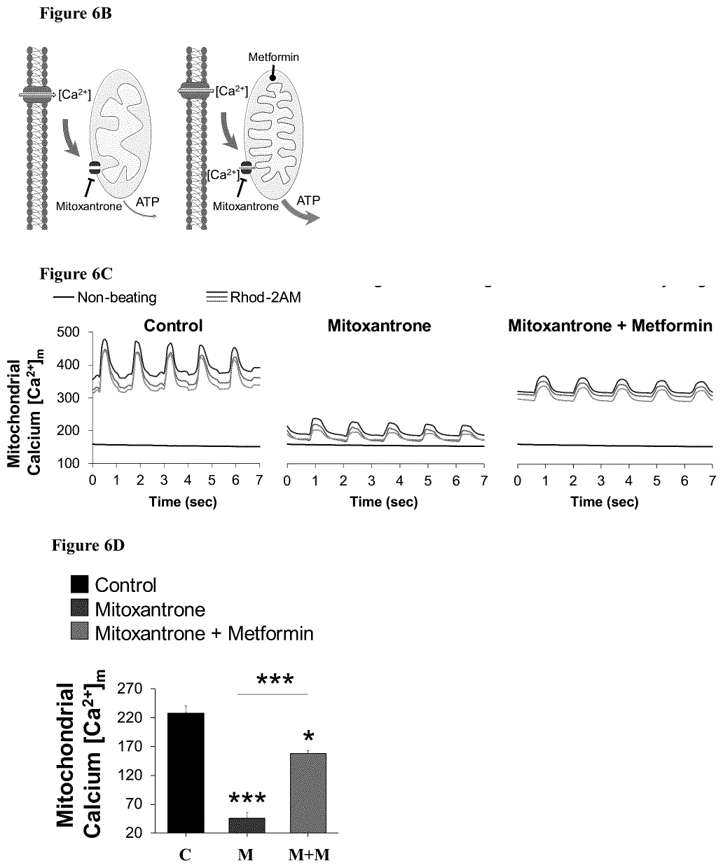

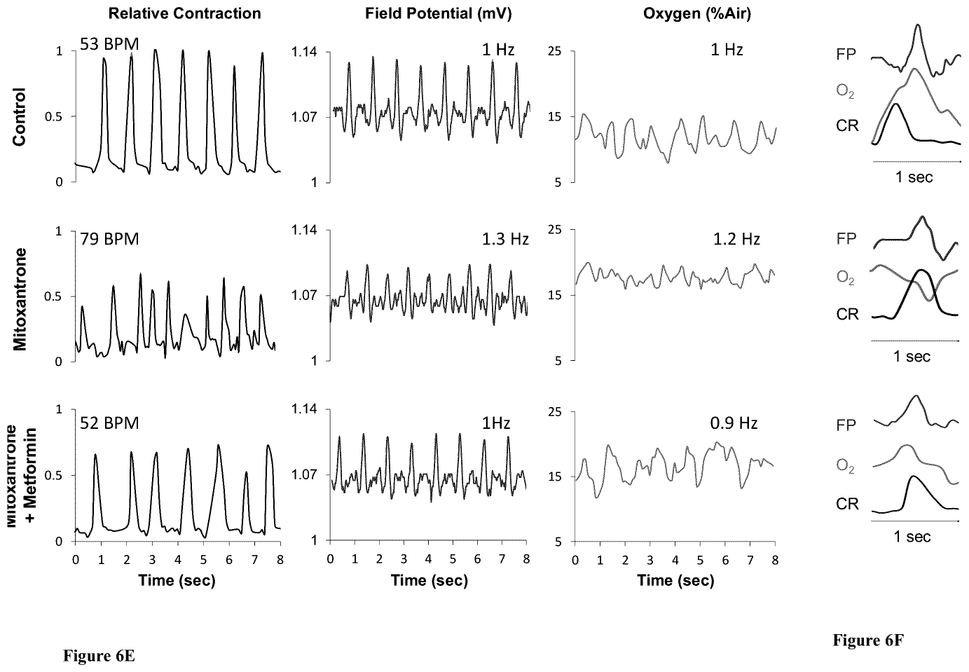

Mitoxantrone is a human topoisomerase II inhibitor used in the treatment of prostate cancer, non-Hodgkin's lymphomas, and multiple sclerosis. Mitoxantrone is one of several chemotherapies thought to be involved in cancer treatment induced arrhythmia (CTIA). Similar cardiovascular risks also limit its use in multiple sclerosis. Recent studies identified mitoxantrone as a selective inhibitor of the MCU (FIG. 6C-6D).

Metformin was reported to increase mitochondrial calcium and MCU activity suggesting it could reverse this effect. Indeed, exposure to metformin increased mitochondrial calcium concentration of mitoxantrone-treated cells by 3.4-fold (n=4, p<0.001; FIG. 6A-6D).

To demonstrate this behavior in a physiological model the inventors exposed cardiac organoids cultured on the herein disclosed sensor-integrated platform to mitoxantrone or mitoxantrone and metformin combination (FIGS. 6E-6F). Mechanical contraction, electrical depolarization, and oxygen concentration markedly decreased due to mitoxantrone exposure (FIG. 6E). Irregular contraction and abnormal field potential were associated with a disruption of the electro-mitochondrial rhythm (FIG. 6F). The contraction rate increased from 53 to 79 BPM (FIG. 6E-6F), collectively indicating arrhythmogenic behavior. The combination of mitoxantrone with metformin reversed this effect (FIG. 6E-6F). Organoid contraction became regular, and dominant electrical field potential frequency was restored. Moreover, oxygen consumption increased and the correlation between the electro and mitochondrial rhythm was restored (FIGS. 6E-6F). The beating rate decreased from 82 to 61 BPM restoring normal cardiac organoid activity (FIG. 6E).

### Example 9

### Validation of Electro-Mitochondrial Coupling in an Ex-Vivo Porcine Model

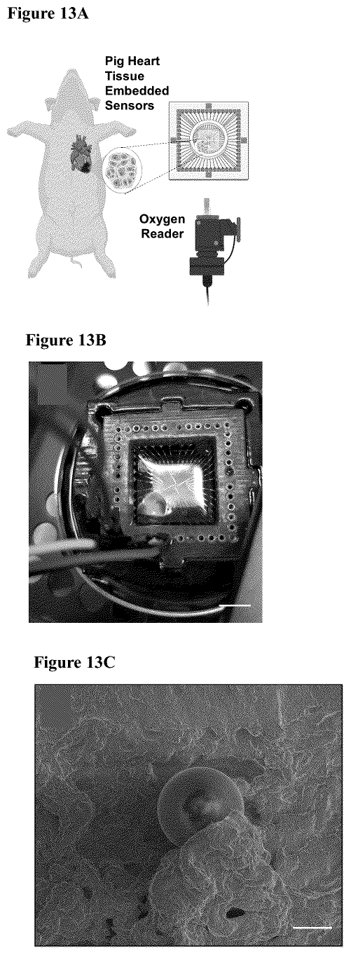

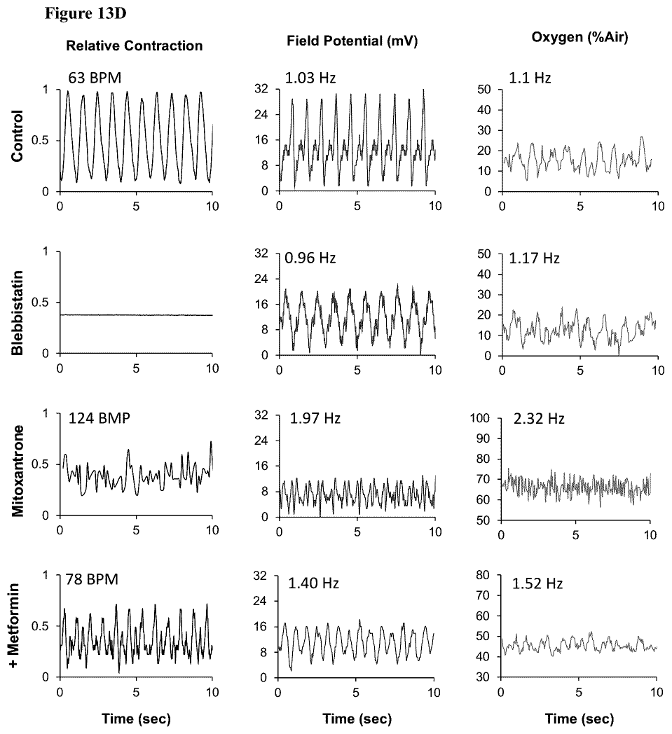

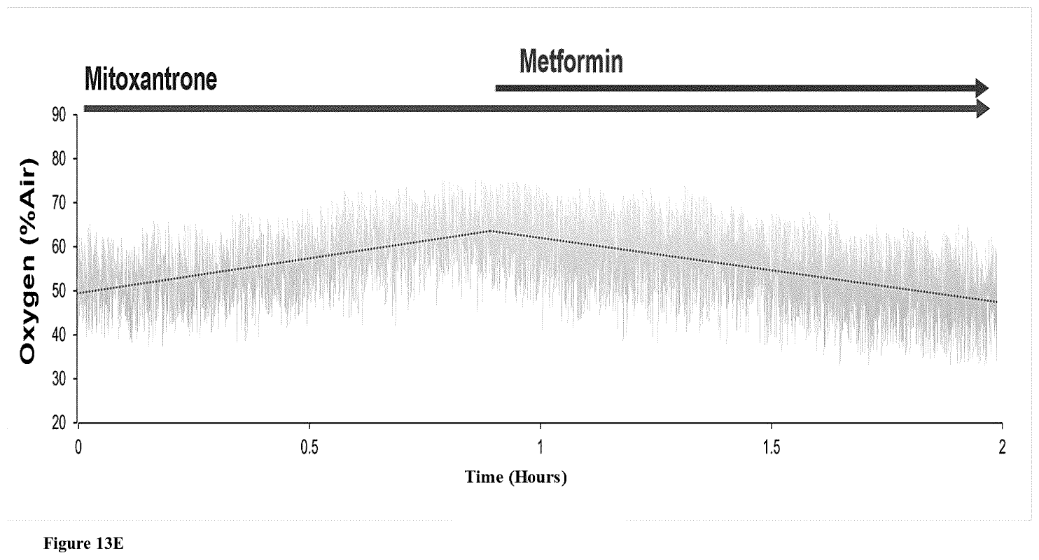

Previous work demonstrated the use of cardiac tissue sections from adult porcine models is a physiologically relevant model for human ex vivo validation as it shares physiological and electrical characteristics with human hearts. 500±200 μm thick porcine myocardial tissue was surgically prepared from the left ventricle of a 70 kg female porcine heart, embedded with oxygen sensors, and placed on the MEA chip (FIG. 13A-C). Contraction, field potential, and interstitial oxygen of the myocardial slices were measured using the integrated metabolic-electro-mechanical sensor chip during a 2 Hz point-stimulation (see Materials and methods).

Porcine ex vivo myocardial tissue showed the same oxygen oscillations and responses recorded in human cardiac organoids (FIG. 13D). Exposure to myosin II inhibitor blebbistatin blocked mechanical contraction without affecting field potential or oxygen oscillations. While exposure to the MCU inhibitor mitoxantrone decreased oxygen consumption and doubled the cardiac rhythm from 1 to 2 Hz (FIG. 13D). Concurrent treatment of mitoxantrone and metformin restored beating frequency to 1.4 Hz, field potential, and oxygen oscillations (FIG. 13D).

Furthermore, metformin added 50 minutes after exposure to mitoxantrone, gradually restored oxygen consumption over 1 hour (FIG. 13E). These findings validate our earlier results in human cardiac organoids, connecting the electro-mitochondrial desynchronization to arrhythmogenic behavior (FIG. 6A-F).

### DISCUSSION

Microphysiological systems emulate critical aspects of human physiology, but their true potential lies in the ability to discover new physiological mechanisms in a controlled environment. In this work, we developed a sensor-based platform that can correlate mechanical-electrical and metabolic activity in human cardiac organoids. Using this platform, the inventors showed an electro-mitochondrial axis associated with the human cardiac rhythm. Disruption of this electro-mitochondrial rhythm led to arrhythmia, which could be partly corrected by the energy disruptor metformin.

Early work on small animal models showed that oxygen consumption was correlated to the heart mechanical activity, while other research showed that the cardiac electrical and mechanical activities were coupled. Further improvements in electrophysiological methods allowed several groups to connect oscillations in mitochondrial calcium to electromechanical rhythms, suggesting the presence of a cardiac metabolic-electro-mechanical axis in animals. Current understanding is that actomyosin mechanics produce rhythmic depletion in ATP and calcium, driving a metabolic cycle. Advances in imaging techniques allowed to the detection of waves of mitochondrial membrane potential in guinea pig ventricular cells, demonstrating slow oscillations in glycolysis and mitochondrial activity during the cardiac cycle. Others demonstrated similar oscillations in rat cardiomyocytes. These studies strongly suggest that the metabolism of human cardiomyocytes may be similarly driven to oscillate by cardiac contractions.

Regretfully, ion channel dynamics, contraction rate, and lipid metabolism fundamentally differ between small animal models and humans leading to species-specific responses to pathological events on the molecular and metabolic levels. In this context, cardiac organoid technology offers human-relevant metabolism with more mature function than hiPSC-derived cardiomyocytes.

The inventors seeded hiPSC-derived cardiomyocytes in geometrically confined microwells, previously shown to promote the formation of endocardium-coated microchamber through WNT/β-catenin signaling. In this work, the inventors used cardiac endothelial cells, creating anisotropic stress that drove the formation of multi-chamber organoids. Pacemaker-like clusters, endocardial-like cells, and an epicardial-like shell add to the complexity of these multi-chamber cardiac organoids (FIG. 1).

One advantage of confining organoids to individual microwells is the ability to minimize electrical interference (FIG. 3C). Similarly, simultaneous measurement of background fluorescence allowed the inventors to reduce ambient noise permitting real-time detection of oxygen consumption with 10 Hz resolution (FIG. 8A). Comparing the two signals also allowed the inventors to precisely monitor organoid mechanical movement, resulting in a harmonized measurement of cardiac function. Using this approach the inventors demonstrated 1 Hz oscillations in mitochondrial function, the fastest switch in metabolic flux recorded to date. Metabolic flux balance analysis shows that ATP is primarily generated during cardiac relaxation (FIG. 2F).

However, in contrast to rodents, the actomyosin mechanics of human cardiac organoids does not seem to drive this metabolic oscillation, as electrical and metabolic rhythms cycle even in the absence of mechanical activity (FIG. 3A-L). Instead, this electro-metabolic coupling is driven by [Ca]m oscillations in human cardiomyocytes (FIG. 4A-G) as muscle depolarization leads to increased mitochondrial activity in preparation for the spike in energy demand of mechanical contraction. This electro-mitochondrial coupling is facilitated by the mitochondrial calcium uniporter (MCU). Mitochondrial calcium uniporters (MCU) were shown to play a role in cardiac homeostasis of rodents. MCU^{-/-} knockout mice were unable to increase heart rate during stress and were more susceptible to ischemic injury. The current findings support those early observations showing even a mild inhibition of MCU activity in human cardiac organoids, results in loss of electro-mitochondrial synchronization and arrhythmogenic behavior (FIGS. 5A-E and 6A-F). Indeed, loss of electro-metabolic synchronization may underlie some cases of catastrophic arrhythmias occurring following ischemic injury, viral infection, or cancer treatment-induced arrhythmia.

Mitoxantrone is one of several chemotherapies thought to be involved in cancer treatment-induced arrhythmia (CTIA). Indeed, many drugs beyond just cancer treatments are known to induce arrhythmia and other cardiac side effects (see Table 1). Recent studies identified mitoxantrone as a selective inhibitor of the MCU. Exposure of human cardiac organoids to mitoxantrone caused a disruption of the electro-mitochondrial rhythm, leading to irregular contraction, abnormal field potential, and increase heart rate (FIGS. 6A-F and 7A-D). Metformin is an energy disruptor recently shown to increase mitochondrial calcium and MCU activity. The addition of metformin partly restored the electro-mitochondrial coupling, resolving arrhythmia, in both human organoids and the ex-vivo model of porcine myocardium (FIGS. 6A-F and 7A-D).

The current work illuminates a new physiological mechanism that may provide novel targets for the development of antiarrhythmic therapies. Further studies are needed to elucidate the precise molecular mechanism of electro-mitochondrial coupling and demonstrate its role in adult human tissue. Indeed, observational studies are required to delineate the interplay between metformin and cancer medications, with further placebo-controlled studies to elucidate the exact role of such interventions in different conditions. The current findings underscore the utility of microphysiological systems in the study of human physiology and suggest that novel metamaterials cells and sensors could go beyond the utility of animal models.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. (canceled)

2. (canceled)

3. (canceled)

4. (canceled)

5. (canceled)

6. (canceled)

7. (canceled)

8. (canceled)

9. (canceled)

10. (canceled)

11. A multichambered cardiac organoid comprising cardiomyocytes and endothelial cells and at least two chambers beating in synchrony.

12. The organoid of claim 11, wherein all chambers beat in synchrony, said organoid produces a biphasic beating, said synchronized beating comprises a basal beating frequency of between 50 and 90 beats per minute (bpm), or a combination thereof.

13. The organoid of claim 11, comprising pacemaker-like cell clusters, optionally wherein said pacemaker-like cell clusters are Potassium/sodium hyperpolarization-activated cyclic nucleotide-gated channel 4 (HCN4) and Short-stature homeobox 2 (SHOX2) positive.

14. The organoid of claim 11, comprising an outer epicardium, optionally wherein said epicardium comprises cells positive for Wilms' tumor-1 (WT1) and T-box transcription factor 18 (TBX18).

15. The organoid of claim 11, comprising an inner endocardium, optionally wherein said endocardium comprises cells positive for Platelet endothelial cell adhesion molecule (PECAM-1).

16. (canceled)

17. The organoid of claim 11, wherein said organoid comprises vascular structures, circumferentially aligned cardiomyocytes surrounding hollow chambers, elongated cardiomyocytes organized in a sarcomeric pattern, capillaries within a wall of said chambers and cardiac fibroblast-like cells, optionally wherein said fibroblast-like cells are Periostin (POSTN) and/or Vimentin positive.

18. The organoid of claim 11, wherein said organoid comprises at least one parameter that is increased as compared to isolated cardiomyocytes in culture or fetal cardiac tissue in culture, wherein said parameter is selected from basal respiration, oxidative phosphorylation, mitochondrial maximal capacity and expression of at least factor selected from the group consisting of: TNNT2, TNNI3, Cx43, MYH7, AKAP6, GJA5, JPH2, SLC8A1, ATP2A2, CACNA1C, RYR2, CASQ2, PLN, CAMK2B, TRDN, CAV3, BIN1, AMP2, SCN5A, KIR2.1, ITPR3, HCN2, SCN1B, HCN1, KCNJ8, KCNH2, PRKAA1, CPT1A, TFAM, PPARGC1A, PPA1, PPP2R4, SLC2A4, MAPK1, PRKACA, α1A, α1B, SCN4B, KCNE1.

19. A method of producing a multichambered cardiac organoid comprising at least two chambers beating in synchrony, the method comprising coculturing a mass of cardiomyocytes and endothelial cells in a geometrically confined culture space such that anisotropic stress gradients are generated in said cell mass, thereby producing a multichambered cardiac organoid.

20. The method of claim 19, comprising culturing about 6.8×10\hat{ }4 cells in a microwell comprising a diameter of between 1-1.2 mm.

21. The method of claim 19, wherein said coculture comprises a ratio of cardiomyocytes to endothelial cells of between 1.5:1 and 2.5:1.

22. A multichambered cardiac organoid comprising at least two chambers beating in synchrony produced by a method of claim 19.

23. A method of evaluating cardiac cell function, the method comprising exposing a multichambered cardiac organoid of claim 11 to a condition and measuring at least one parameter of said multichambered cardiac organoid, optionally wherein said condition is selected from: application of a drug or chemical, hypoxic conditions, circulation conditions, change in metabolite exposure, change in hormone exposure, and genetic mutation of cells in said organoid.

24. (canceled)

25. The method of claim 23, wherein said at least one parameter is electro-mitochondrial synchronization.

26. A sensing system comprising:
an illumination source;
a first photomultiplier tube (PMT) sensor;
a second PMT sensor and
a controller configured to:
control said illumination source to illuminate a microparticle embedded in a tissue or cell aggregate with a photon beam having a first wavelength;
detect, by said first PMT sensor, a first signal indicative of photons reflected from said microparticle at said first wavelength;
detect, by said second PMT sensor, a second signal indicative of emission from the microparticles at a second wavelength, wherein said microparticles comprise an excitable molecule quenchable by a cofactor;
measure a shift between a frequency of said first signal and a frequency of said photon beam, determine background noise based on said measured shift and reduce background noise from said second signal; and
calculate temporal cofactor consumption of said tissue or cell aggregate based on said background noise-reduced second signal.

27. The system of claim 26, wherein said temporal cofactor consumption is indicative of the oxygen level in said tissue or cell aggregate.

28. The system of claim 26, wherein said controller is further configured to
a. detect a change in intensity of said first signal and calculate relative displacement of said microparticle, based on said detected change;
b. sense field potential of said tissue or cell aggregate from an array of microelectrodes for measuring the electrical activity of the tissue or cell aggregate simultaneously to detecting said first signal and said second signal; or
c. both.

29. The system of claim 28, wherein said detected changes in the intensity of the signal is indicative of the relative displacement of said microparticle, optionally wherein said displacement is measured in an axis perpendicular to the photons beam.

30. (canceled)

31. A method of evaluating cellular function, the method comprising:
a. placing tissue, an organoid or a cellular aggregate in a sensing system of claim 26,
b. applying a condition to said tissue, organoid or cellular aggregate, optionally wherein said applying a condition is selected from: application of a drug or chemical, application of hypoxic conditions, application of circulation conditions, changing metabolite exposure, changing hormone exposure, and genetic mutation of cells in said tissue, organoid or aggregate; and
c. measuring at least cofactor consumption in said tissue, organoid or cellular aggregate,
thereby evaluating cellular function.

32. The method of claim 31, wherein said controller of said sensing system is further configured to sense field potential of said tissue or cell aggregate from an array of microelectrodes for measuring the electrical activity of the tissue or cell aggregate simultaneously to detecting said first signal and said second signal, and said measuring comprises measuring cofactor consumption, displacement and electrical field potential in said tissue, organoid or cellular aggregate and wherein a significant deviation in displacement, cofactor consumption, and electrical field potential after applying said condition as compared to displacement, cofactor consumption, and electrical field potential before applying said condition or as compared to control untreated tissue, organoid or cellular aggregate is indicative of electro-mitochondrial desynchronization.

33. The method of claim 31, wherein a cardiac or brain organoid is placed in said sensing system.

34. (canceled)

35. (canceled)

36. (canceled)


SEQUENCE LISTING (4 sequences)

SEQ ID NO: 1 - DNA, 20 bp, synthetic construct
  source 1..20: mol_type = other DNA
ggcccccgcgctttaaaata

SEQ ID NO: 2 - DNA, 20 bp, synthetic construct
  source 1..20: mol_type = other DNA
ggaaagcgctggtagaagcc

SEQ ID NO: 3 - DNA, 20 bp, synthetic construct
  source 1..20: mol_type = other DNA
cacacagtttggcattttgg

SEQ ID NO: 4 - DNA, 20 bp, synthetic construct
  source 1..20: mol_type = other DNA
cgtgactttttggctccttt